SEQUENCE LISTING (42 sequences)

SEQ ID NO: 1 - protein, 9 aa, synthetic construct
  REGION 1..9: note = peptide
SLPDRILTV

SEQ ID NO: 2 - protein, 9 aa, synthetic construct
  REGION 1..9: note = peptide
ALNPELLAL

SEQ ID NO: 3 - protein, 9 aa, synthetic construct
  REGION 1..9: note = peptide
FLYDVRTYL

SEQ ID NO: 4 - protein, 9 aa, synthetic construct
  REGION 1..9: note = peptide
YLYDVRTAL

SEQ ID NO: 5 - protein, 9 aa, synthetic construct
  REGION 1..9: note = peptide
SLLSIQSYV

SEQ ID NO: 6 - protein, 9 aa, synthetic construct
  REGION 1..9: note = peptide
YLQQELMNL

SEQ ID NO: 7 - protein, 9 aa, synthetic construct
  REGION 1..9: note = peptide
ALYSEILTV

SEQ ID NO: 8 - protein, 9 aa, synthetic construct
  REGION 1..9: note = peptide
LILDTVHSL

SEQ ID NO: 9 - protein, 9 aa, synthetic construct
  REGION 1..9: note = peptide
TLDQKLFMV

SEQ ID NO: 10 - protein, 9 aa, synthetic construct
  REGION 1..9: note = peptide
YLILEYATV

SEQ ID NO: 11 - protein, 9 aa, synthetic construct
  REGION 1..9: note = peptide
KIIGIILAV

SEQ ID NO: 12 - protein, 9 aa, synthetic construct
  REGION 1..9: note = peptide
YLSGANLFV

SEQ ID NO: 13 - protein, 9 aa, synthetic construct
  REGION 1..9: note = peptide
IVWSDVTYV

SEQ ID NO: 14 - protein, 9 aa, synthetic construct
  REGION 1..9: note = peptide
AVIGIVAAV

SEQ ID NO: 15 - protein, 9 aa, synthetic construct
  REGION 1..9: note = peptide
LLNSTTIPI

SEQ ID NO: 16 - protein, 9 aa, synthetic construct
  REGION 1..9: note = peptide
ALIFGQLLL

SEQ ID NO: 17 - protein, 9 aa, Homo sapiens
SLPDRILDA

SEQ ID NO: 18 - protein, 9 aa, Homo sapiens
AINPELLQL

SEQ ID NO: 19 - protein, 9 aa, Homo sapiens
ALYDVRTIL

SEQ ID NO: 20 - protein, 9 aa, Homo sapiens
ILLSIQSLL

SEQ ID NO: 21 - protein, 9 aa, Homo sapiens
RLQQELMTL

SEQ ID NO: 22 - protein, 9 aa, Homo sapiens
AVYSEILSV

SEQ ID NO: 23 - protein, 9 aa, Homo sapiens
KILDTVHSC

SEQ ID NO: 24 - protein, 9 aa, Homo sapiens
SLDQKLFQL

SEQ ID NO: 25 - protein, 9 aa, Homo sapiens
YLILEYAPL

SEQ ID NO: 26 - protein, 9 aa, Homo sapiens
LVIGIILAV

SEQ ID NO: 27 - protein, 9 aa, Homo sapiens
YLSGANLNL

SEQ ID NO: 28 - protein, 9 aa, Homo sapiens
KVWSDVTPL

SEQ ID NO: 29 - protein, 9 aa, Homo sapiens
AQIGIVAVV

SEQ ID NO: 30 - protein, 9 aa, Homo sapiens
ILNSTTIEI

SEQ ID NO: 31 - protein, 9 aa, Homo sapiens
ALIFGQLLT

SEQ ID NO: 32 - protein, 9 aa, synthetic construct
  REGION 1..9: note = peptide
FMLGEFLKL

SEQ ID NO: 33 - protein, 9 aa, synthetic construct
  REGION 1..9: note = peptide
LMDLSTTEV

SEQ ID NO: 34 - protein, 9 aa, synthetic construct
  REGION 1..9: note = peptide
RLSSYLVEI

SEQ ID NO: 35 - protein, 9 aa, Homo sapiens
LTLGEFLKL

SEQ ID NO: 36 - protein, 9 aa, Homo sapiens
LMDLSTTPL

SEQ ID NO: 37 - protein, 9 aa, Homo sapiens
RVSSYLVPI

SEQ ID NO: 38 - protein, 10 aa, Homo sapiens
ALYDVRTILL

SEQ ID NO: 39 - protein, 15 aa, synthetic construct
  REGION 1..15: note = UCP2 helper peptide
KSVWSKLQSIGIRQH

SEQ ID NO: 40 - protein, 9 aa, synthetic construct
  source 1..9: note = peptide
QMNPELLTL

SEQ ID NO: 41 - protein, 9 aa, synthetic construct
  source 1..9: note = peptide
VMNPELLLL

SEQ ID NO: 42 - protein, 9 aa, synthetic construct
  source 1..9: note = peptide
VLNPELLML

(19) United States
(12) Patent Application Publication
(10) Pub. No.: US 2025/0205324 A1
(43) Pub. Date: Jun. 26, 2025

(54) ANTIGENIC PEPTIDES FOR PREVENTION AND TREATMENT OF CANCER

(71) Applicant: ENTEROME S.A., Paris (FR)

(72) Inventors: Laurent CHENE, NEUVILLE AUX BOIS (FR); Joao Gamelas MAGALHAES, CORBEIL ESSONNES (FR); Alice TALPIN, ALFORTVILLE (FR); Guillaume KULAKOWSKI, ANTONY (FR); Francesco STROZZI, PARIS (FR); Camille GAAL, Saint Jean Le Blanc (FR)

(21) Appl. No.: 18/852,118

(22) PCT Filed: Mar. 30, 2023
(86) PCT No.: PCT/EP2023/058415

§ 371 Date: Sep. 27, 2024

(30) Foreign Application Priority Data
Mar. 31, 2022 (EP) 22305420.6

(51) Int. Cl. A61K 39/00; A61K 47/64; C07K 7/06

(52) U.S. Cl. CPC A61K 39/001114; A61K 47/646; C07K 7/06; A61K 2039/55516; A61K 2039/572

(57) ABSTRACT

The present invention relates to antigen-based immunotherapy, in particular cancer immunotherapy. In particular, the present invention provides antigenic peptides, which are distinct from, but have amino acid similarity to, fragments of human tumor antigens. The present invention further provides immunogenic compounds, nanoparticles, cells and pharmaceutical compositions comprising such antigenic peptides and nucleic acids encoding such antigenic peptides.

The present invention relates to the field of cancer therapy, more particularly to immunotherapeutic methods. In particular, the present invention provides various peptides, which are useful in cancer immunotherapy.

Cancer is one of the leading causes of death across the world. According to the World Health Organization (WHO), in 2012 only, 14 million new cases and 8.2 million cancer-related deaths were reported worldwide, and it is expected that the number of new cancer cases will rise by about 70% within the next two decades. So far, more than 60% of world's total new annual cases occur in Africa, Asia and Central and South America. These regions also account for 70% of the world's cancer deaths. Among men, the five most common sites of cancer are lung, prostate, colorectum, stomach and liver; while in women, those are breast, colorectum, lung, cervix, and stomach.

Cancer has long been managed with surgery, radiation therapy, cytotoxic chemotherapy, and endocrine manipulation, which are typically combined in sequential order so as to best control the disease. However, major limitations to the true efficacy of these standard therapies are their imprecise specificity which leads to the collateral damage of normal tissues incurred with treatment, a low cure rate, and intrinsic drug resistance.

In the last years, there has been a tremendous increase in the development of cancer therapies due notably to great advances in the expression profiling of tumors and normal cells, and recent research and first clinical results in immunotherapy, or molecular targeted therapy, have started to change our perception of this disease.

Promising anticancer immunotherapies have now become a reality and evidences that the host immune system can recognize tumor antigens have led to the development of anticancer drugs which are now approved by regulatory agencies as the US Food and Drug Administration (FDA) and European Medicines Agency (EMA). Various therapeutic approaches include, among others, adoptive transfer of ex vivo expanded tumor-infiltrating lymphocytes (TIL), cancer cell vaccines, immunostimulatory cytokines and variants thereof, Pattern recognition receptor (PRR) agonists, and immunomodulatory monoclonal antibodies targeting tumor antigens or immune checkpoints (Galuzzi et al., Classification of current anticancer immunotherapies. Oncotarget. 2014 Dec. 30; 5(24):12472-508).

Unfortunately, a significant percentage of patients can still present an intrinsic resistance to some of these immunotherapies or even acquire resistance during the course of treatment. For example, the three-year survival rate has been reported to be around 20% with the anti-CTLA-4 antibody Ipilimumab in unresectable or metastatic melanoma (Snyder et al., Genetic basis for clinical response to CTLA-4 blockade in melanoma. N Engl J Med. 2014 Dec. 4; 371(23):2189-2199; Schadendorf et al., Pooled Analysis of Long-Term Survival Data From Phase II and Phase III Trials of Ipilimumab in Unresectable or Metastatic Melanoma. J Clin Oncol. 2015 Jun. 10; 33(17):1889-94), while the three-year survival rate with another checkpoint inhibitor, Nivolumab targeting PD-1, has been reported to be of 44% in renal cell carcinoma (RCC) and 18% in non-small-cell lung carcinoma (NSCLC) (Mc Dermott et al., Survival, Durable Response, and Long-Term Safety in Patients With Previously Treated Advanced Renal Cell Carcinoma Receiving Nivolumab. J Clin Oncol. 2015 Jun. 20; 33(18):2013-20; Gettinger et al., Overall Survival and Long-Term Safety of Nivolumab (Anti-Programmed Death 1 Antibody, BMS-936558, ONO-4538) in Patients With Previously Treated Advanced Non-Small-Cell Lung Cancer. J Clin Oncol. 2015 Jun. 20; 33(18):2004-12). Fundamental drug resistance thus represents a fixed barrier to the efficacy of these immunotherapies. It is thus clear that a different approach to cancer treatment is needed to break this barrier.

Absence of response in a large number of subjects treated with these immunotherapies might be associated with a deficient anti-tumor immune response (as defect in antigen presentation by antigen-presenting cells (APC) or antigen recognition by T cells). In other words, positive response to immunotherapy correlates with the ability of the immune system to develop specific lymphocytes subsets able to recognize MHC class I-restricted antigens that are expressed by human cancer cells (Kvistborg et al., Human cancer regression antigens. Curr Opin Immunol. 2013 April; 25(2):284-90). This hypothesis is strongly supported by data demonstrating that response to adoptive transfer of tumor-infiltrating lymphocytes (TIL), is directly correlated with the numbers of CD8⁺ T-cells transfused to the patient (Besser et al., Adoptive transfer of tumor-infiltrating lymphocytes in patients with metastatic melanoma: intent-to-treat analysis and efficacy after failure to prior immunotherapies. Clin Cancer Res. 2013 Sep. 1; 19(17):4792-800). A potent anti-tumoral response will thus depend on the presentation of immunoreactive peptides and the presence of a sufficient number of reactive cells “trained” to recognize these antigens.

Tumor antigen-based vaccination represent a unique approach to cancer therapy that has gained considerable interest as it can enlist the patient's own immune system to recognize, attack and destroy tumors, in a specific and durable manner. Tumor cells are indeed known to express a large number of peptide antigens susceptible to be recognized by the immune system. Vaccines based on such antigens thus provide great opportunities not only to improve patient's overall survival but also for the monitoring of immune responses and the preparation of GMP-grade product thanks to the low toxicity and low molecular weight of tumor antigens. Examples of tumor antigens include, among others, by-products of proteins transcribed from normally silent genes or overexpressed genes and from proteins expressed by oncovirus (Kvistborg et al., Human cancer regression antigens. Curr Opin Immunol. 2013 April; 25(2):284-90), and neo-antigens, resulting from point mutations of cellular proteins. The latter are of particular interest as they have been shown to be directly associated with increased overall survival in patient treated with CTLA-4 inhibitors (Snyder et al., Genetic basis for clinical response to CTLA-4 blockade in melanoma. N Engl J Med. 2014 Dec. 4; 371(23):2189-2199; Brown et al., Neo-antigens predicted by tumor genome meta-analysis correlate with increased patient survival. Genome Res. 2014 May; 24(5):743-50).

Nevertheless, the number of human tumor antigens on which cancer vaccines can be developed is limited. In particular, antigens derived from mutated or modified self-proteins may induce immune tolerance and/or undesired autoimmunity side effects.

There is thus a need in the art to identify alternative cancer therapeutics, which can overcome the limitations encountered in this field.

The invention has for objective to meet the aforementioned needs. This object is achieved by means of the subject-matter set out below, in particular in the items provided by the present invention and in the appended claims.

### Items of the Invention

In particular, the present invention provides in particular the following items:

1. An antigenic peptide comprising or consisting of an amino acid sequence as set forth in any one of SEQ ID NOs 1-16 and SEQ ID NOs 40-42.

2. An antigenic peptide comprising or consisting of an amino acid sequence as set forth in any one of SEQ ID NOs 1-16 and SEQ ID NOs 40-42 wherein, optionally, one or two amino acid residues may be substituted, deleted or added.

3. The antigenic peptide according to item 2, wherein the core sequence is maintained.

4. The antigenic peptide according to any one of the previous items, wherein the antigenic peptide comprises or consists of a microbiota variant of a human reference peptide according to any one of SEQ ID NOs 17-31.

5. The antigenic peptide according to any one of the previous items, wherein the antigenic peptide consists of an amino acid sequence as set forth in any one of SEQ ID NOs 1-16 and SEQ ID NOs 40-42.

6. The antigenic peptide according to any one of the previous items, wherein the antigenic peptide has a length of 8 to 15 amino acids or of 8 to 11 amino acids, preferably the antigenic peptide has a length of 9 or 10 amino acids.

7. The antigenic peptide according to any one of the previous items, wherein the antigenic peptide comprises or consists of an amino acid sequence as set forth in SEQ ID NO: 1.

8. The antigenic peptide according to any one of the previous items, wherein the antigenic peptide comprises or consists of an amino acid sequence as set forth in SEQ ID NO: 2.

9. The antigenic peptide according to any one of the previous items, wherein the antigenic peptide comprises or consists of an amino acid sequence as set forth in SEQ ID NO: 3.

10. The antigenic peptide according to any one of the previous items, wherein the length of the antigenic peptide does not exceed 30 amino acids.

11. The antigenic peptide according to any one of the previous items, wherein the length of the antigenic peptide does not exceed 25 amino acids.

12. The antigenic peptide according to any one of the previous items, wherein the length of the antigenic peptide does not exceed 20 amino acids.

13. The antigenic peptide according to any one of the previous items, wherein the length of the antigenic peptide does not exceed 15 amino acids.

14. The antigenic peptide according to any one of the previous items, wherein the length of the antigenic peptide does not exceed 11 amino acids.

15. The antigenic peptide according to any one of the previous items, wherein the antigenic peptide is not a full-length (microbiota) protein.

16. An immunogenic compound comprising the antigenic peptide according to any one of the previous items.

17. The immunogenic compound according to item 16, wherein the antigenic peptide is linked to a carrier molecule.

18. The immunogenic compound according to item 17, wherein the carrier molecule is a carrier protein or a carrier peptide.

19. The immunogenic compound according to any one of items 16-18 comprising or consisting of a polypeptide of formula (I)

PepNt-CORE-PepCt (I)
wherein:
   “PepNt” consists of a polypeptide having a length varying from 0 to 500 amino acid residues and is located at the N-terminal end of the polypeptide of formula (I);
   CORE consists of an antigenic peptide as defined in any one of items 1-15; and
   “PepCt” consists of a polypeptide having a length varying from 0 to 500 amino acid residues and is located at the C-terminal end of the polypeptide of formula (I).

20. A nanoparticle loaded with
at least one of the antigenic peptides according to any one of items 1-15, or
at least one of the immunogenic compounds according to any one of items 16-19; and, optionally, with an adjuvant.

21. A cell loaded with the antigenic peptide according to any one of items 1-15 or with the immunogenic compound according to any one of items 16-19.

22. The cell according to item 38, wherein said cell is an antigen presenting cell, preferably a dendritic cell.

23. A nucleic acid encoding the antigenic peptide according to any one of items 1-15, the polypeptide of formula (I) as defined in item 19, or the immunogenic compound according to any one of items 16-19, wherein the immunogenic compound is a peptide or a protein.

24. The nucleic acid according to item 23, wherein the nucleic acid is a DNA molecule or an RNA molecule; preferably selected from genomic DNA; cDNA; siRNA; rRNA; mRNA; antisense DNA; antisense RNA; ribozyme; complementary RNA and/or DNA sequences; RNA and/or DNA sequences with or without expression elements, regulatory elements, and/or promoters; a vector; and combinations thereof.

25. A host cell comprising the nucleic acid according to item 23 or 24.

26. The host cell according to item 25, wherein the nucleic acid is a vector.

27. The host cell according to item 25 or 26, wherein the host cell is a bacterial cell, preferably a gut bacterial cell.

28. A (cytotoxic and/or activated) T lymphocyte specific for an antigenic peptide according to any one of items 1-15.

29. An antibody binding to an antigenic peptide according to any one of items 1-15.

30. A T cell receptor binding to an antigenic peptide according to any one of items 1-15.

31. A pharmaceutical composition comprising
the antigenic peptide according to any one of items 1-15,
the immunogenic compound according to any one of items 16-19,
the nanoparticle according to item 20,
the cell according to item 21 or 22,
the nucleic acid according to item 23 or 24,
the host cell according to any one of items 25-27,
the T lymphocyte according to item 28,
the antibody according to item 29, or
the T cell receptor according to item 30,
and, optionally, one or more pharmaceutically acceptable excipients or carriers.

32. The pharmaceutical composition according to item 31, wherein the composition comprises
(i) at least two distinct antigenic peptides according to any one of items 1-15;
(ii) at least two distinct immunogenic compounds according to any one of items 16-19;
(iii) at least two distinct nanoparticles according to item 20;
(iv) at least two distinct nucleic acids according to item 21 or 22; or
(v) at least two distinct cytotoxic T lymphocytes according to item 28.

33. The pharmaceutical composition according to item 32 comprising at least three or four distinct components according to any one of (i)-(v), preferably three or four distinct antigenic peptides.

34. The pharmaceutical composition according to item 33, wherein the at least three or four distinct active components relate to
the antigenic peptide comprising or consisting of an amino acid sequence as set forth in SEQ ID NO: 1;
the antigenic peptide comprising or consisting of an amino acid sequence as set forth in SEQ ID NO: 2; and
the antigenic peptide comprising or consisting of an amino acid sequence as set forth in SEQ ID NO: 3.

35. The pharmaceutical composition according to any one of items 31-34, wherein the pharmaceutical composition further comprises
the antigenic peptide comprising or consisting of an amino acid sequence as set forth in SEQ ID NO: 32; and
the antigenic peptide comprising or consisting of an amino acid sequence as set forth in SEQ ID NO: 33, or the antigenic peptide comprising or consisting of an amino acid sequence as set forth in SEQ ID NO: 34.

36. The pharmaceutical composition according to any one of items 31-35 further comprising a helper peptide, preferably the peptide comprising or consisting of an amino acid sequence according to SEQ ID NO: 39.

37. A kit comprising
the antigenic peptide according to any one of items 1-15,
the immunogenic compound according to any one of items 16-19,
the nanoparticle according to item 20,
the cell according to item 21 or 22,
the nucleic acid according to item 23 or 24,
the host cell according to any one of items 25-27,
the T lymphocyte according to item 28,
the antibody according to item 29,
the T cell receptor according to item 30, or
the pharmaceutical composition according to any one of items 31-36.

38. The kit according to item 37 further comprising a package insert or instruction leaflet with directions to prevent or to treat a cancer by using the antigenic peptide, the immunogenic compound, the nanoparticle, the cell, the nucleic acid, the host cell, the cytotoxic T lymphocyte and/or the pharmaceutical composition.

39. The kit according to item 37 or 38, wherein the kit comprises at least two distinct antigenic peptides according to any one of items 1-15.

40. A combination of at least two distinct antigenic peptides according to any one of items 1-15 for use in the prophylaxis or treatment of cancer.

41. An antigenic peptide according to any one of items 1-15 for use as medicament.

42. An antigenic peptide according to any one of items 1-15 for use in the prophylaxis or treatment of cancer.

43. The antigenic peptide according to any one of items 1-15,
the immunogenic compound according to any one of items 16-19,
the nanoparticle according to item 20,
the cell according to item 21 or 22,
the nucleic acid according to item 23 or 24,
the host cell according to any one of items 25-27,
the T lymphocyte according to item 28,
the antibody according to item 29,
the T cell receptor according to item 30,
the pharmaceutical composition according to any one of items 31-36,
the kit according to any one of items 37-39, or
the combination according to item 40,
for use in medicine, in particular in the prophylaxis and/or treatment of cancer.

44. A method for preventing and/or treating a cancer or initiating, enhancing or prolonging an anti-tumor-response against a cancer in a subject in need thereof comprising administering to the subject
the antigenic peptide according to any one of items 1-15,
the immunogenic compound according to any one of items 16-19,
the nanoparticle according to item 20,
the cell according to item 21 or 22,
the nucleic acid according to item 23 or 24,
the host cell according to any one of items 25-27,
the T lymphocyte according to item 28,
the antibody according to item 29,
the T cell receptor according to item 30,
the pharmaceutical composition according to any one of items 31-36,
the kit according to any one of items 37-39, or
the combination according to item 40.

45. A peptide-MHC (pMHC) multimer comprising the antigenic peptide according to any one of items 1-15.

### Definitions

Unless otherwise defined herein, scientific and technical terms used in the present application shall have the meanings that are commonly understood by those of ordinary skill in the art.

Further, unless otherwise required by context, nomenclatures used herein, and techniques of cell and tissue culture are those well-known and commonly used in the art.

Such techniques are fully explained in the literature, such as Owen et al. (Kuby Immunology, 7^{th}, edition, 2013—W. H. Freeman) and Sambrook et al. (Molecular cloning: A laboratory manual 4th edition, Cold Spring Harbor Laboratory Press—Cold Spring Harbor, NY, USA, 2012).

Nevertheless, with respect to the use of different terms throughout the current specification, the following definitions more particularly apply.

The terms “peptide”, “polypeptide”, “protein” and variations of these terms refer to peptides, oligopeptides, polypeptides, or proteins comprising at least two amino acids joined to each other preferably by a normal peptide bond, or, alternatively, by a modified peptide bond, such as for example in the cases of isosteric peptides. The term “(poly)peptide” refers to a peptide and/or to a polypeptide. In particular, the terms “peptide”, “polypeptide” and “protein” refer to a sequential chain of amino acids of any length linked together via peptide bonds (—NHCO—). Peptides, polypeptides and proteins can play a structural and/or functional role in a cell in vitro and/or in vivo. The terms “peptide”, “polypeptide”, “protein” preferably encompass amino acids chains in size ranging from 2 to at least about 1000 amino acid residues. The term “peptide” preferably encompasses herein amino acid chains in size of less than about 30 amino acids, while the terms “polypeptide” and “protein” preferably encompass amino acid chains in size of at least 30 amino acids. The terms “polypeptide” and “protein” are used herein interchangeably. Preferably, the terms “peptide”, “polypeptide”, “protein” also include “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. In particular, a peptide, polypeptide or protein can comprise amino acids other than the 20 amino acids defined by the genetic code in addition to these amino acids, or it can be composed of amino acids other than the 20 amino acids defined by the genetic code. In particular, a peptide, polypeptide or protein in the context of the present invention can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, “protein” in the context of the present invention in particular also include modified peptides, polypeptides and proteins. For example, peptide, polypeptide or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature (Proteins Structure and Molecular Properties (1993) 2nd Ed., T. E. Creighton, New York; Post-translational Covalent Modifications of Proteins (1983) B. C. Johnson, Ed., Academic Press, New York; Seifter et al. (1990) Analysis for protein modifications and nonprotein cofactors, Meth. Enzymol. 182: 626-646 and Rattan et al., (1992) Protein Synthesis: Post-translational Modifications and Aging, Ann NY Acad Sci, 663: 48-62). Accordingly, the terms “peptide”, “polypeptide”, “protein” preferably include for example lipopeptides, lipoproteins, glycopeptides, glycoproteins and the like.

Preferably, a (poly)peptide or protein is a “classical” (poly)peptide or protein, whereby a “classical” (poly)peptide or protein is typically composed of amino acids selected from the 20 amino acids defined by the genetic code, linked to each other by a normal peptide bond.

As well-known in the art, peptides, polypeptides and proteins can be encoded by nucleic acids. The terms “nucleic acid”, “nucleic acid molecule”, “nucleic acid sequence”, “polynucleotide”, “nucleotide sequence” are used herein interchangeable and refer to a precise succession of natural nucleotides (e.g., A, T, G, C and U), or synthetic nucleotides, i.e. to a chain of at least two nucleotides. In particular, the terms “nucleic acid”, “nucleic acid molecule”, “nucleic acid sequence”, “polynucleotide”, “nucleotide sequence” refer to DNA or RNA. Nucleic acids preferably comprise single stranded, double stranded or partially double stranded DNA or RNA, preferably selected from genomic DNA (gDNA), complementary DNA (cDNA), ribosomal DNA (rDNA), and the transcription product of said DNA, such as RNA. Preferred examples of nucleic acids include ribosomal RNA (rRNA), messenger RNA (mRNA); antisense DNA, antisense RNA; complementary RNA and/or DNA sequences, ribozyme, (complementary) RNA/DNA sequences with or without expression elements, a vector; a mini-gene, gene fragments, regulatory elements, promoters, and combinations thereof. Further preferred examples of nucleic acid (molecules) and/or polynucleotides include, e.g., a recombinant polynucleotide, a vector, an oligonucleotide, an RNA molecule such as an rRNA, an mRNA, or a transfer RNA (tRNA), or a DNA molecule as described above. It is thus preferred that the nucleic acid (molecule) is a DNA molecule or an RNA molecule; preferably selected from gDNA; cDNA; rRNA; mRNA; antisense DNA; antisense RNA; complementary RNA and/or DNA sequences; RNA and/or DNA sequences with or without expression elements, regulatory elements, and/or promoters; a vector; and combinations thereof. It is within the skill of the person in the art to determine nucleotide sequences which can encode a specific amino acid sequence.

The (poly)peptides and/or nucleic acids according to the invention may be prepared by any known method in the art including, but not limited to, any synthetic method, any recombinant method, any ex vivo generation method and the like, and any combination thereof. Such techniques are fully explained in the literature as mentioned above.

The term “antigenic peptide” as used herein refers to a peptide, which is prone to induce/elicit, increase, prolong or maintain an immune response in a subject to whom it is administered. In particular, the antigenic peptide is a sequence variant of (a fragment/epitope of) a (human) tumor antigen. In other words, the antigenic peptide is preferably distinct from (a fragment/epitope of) a (human) tumor antigen, but it has preferably amino acid similarity with (a fragment/epitope of) the (human) tumor antigen. Importantly, the antigenic peptide shares the same core sequence with the respective (fragment/epitope of) a (human) tumor antigen. Preferably, the immune response induced/elicited, increased, prolonged or maintained by the antigenic peptide (also) targets the respective (fragment/epitope of) a (human) tumor antigen.

As used herein, the term “tumor antigen” comprises tumor-specific antigens (TSAs) and tumor-associated antigens (TAAs). In general, the term “tumor antigen” or “tumor protein” designates herein an antigenic substance produced in tumor cells, and sometimes also in normal cells, and which can trigger an immune response upon administration in a subject. In humans, those have been classified according to their expression pattern, function or genetic origin, and include without limitation, overexpressed self-antigens (such as BIRC5); cancer-testis (CT) antigens (such as MAGE-1); mutational antigens, also known as neo-antigens (such as mutants from p53); tissue-specific differentiation antigens (such as the melanoma antigens Melan A/MART-1); viral antigens which are expressed by oncoviruses (such as HPV, EBV); oncofetal antigens (such as alpha-fetoprotein AFP and carcinoembryonic antigen CEA); and universal antigens (telomerase).

The term “core sequence”, as used herein, refers to the amino acids in the middle of the sequence (also referred as the “central amino acids” of the sequence), e.g. in the middle of an antigenic peptide and/or a (reference) epitope. Accordingly, the core sequence consists of all amino acids except the two most N-terminal and the two most C-terminal amino acids. For example, in a peptide of nine amino acids (e.g. an antigenic peptide according to the present invention or the respective (fragment/epitope of) a (human) tumor antigen), the five middle amino acids represent the core sequence and alterations may only occur at any of the two N-terminal and the two C-terminal amino acid positions. Accordingly, a “shared core sequence” (or a “maintained” core sequence) usually means that mutations/differences are allowed only in the two most N-terminal and in the two most C-terminal amino acids of the (reference) epitope/sequence.

The term “microbiota”, as used herein, refers to commensal microorganisms found in and on all multicellular organisms studied to date from plants to animals. In particular, microbiota have been found to be crucial for immunologic, hormonal and metabolic homeostasis of their host. Microbiota include bacteria, archaea, protists, fungi and viruses. Accordingly, a “microbiota sequence variant” (or “microbiota variant”) is a sequence variant of a (human) reference sequence (in particular an epitope/a fragment of a human tumor antigen), which occurs in microbiota, such as bacteria (e.g., it may be contained in a microbiota protein, such as a bacterial protein). Preferably, the antigenic peptide of the invention is a microbiota sequence variant (of a reference epitope/fragment of a human B-cell tumor antigen). Accordingly, the antigenic peptide is preferably present (e.g. comprised in) in at least one protein expressed by the human microbiota.

Anatomically, microbiota reside on or within any of a number of tissues and biofluids, including the skin, conjunctiva, mammary glands, vagina, placenta, seminal fluid, uterus, ovarian follicles, lung, saliva, oral cavity (in particular oral mucosa), and the gastrointestinal tract, in particular the gut. In the context of the present invention the microbiota sequence variant is preferably a sequence variant of microbiota of the gastrointestinal tract (microorganisms residing in the gastrointestinal tract), more preferably a sequence variant of microbiota of the gut (microorganisms residing in the gut). Accordingly, it is most preferred that the microbiota sequence variant is a (human) gut bacterial sequence variant (i.e. a sequence variant of bacteria residing in the (human) gut).

While microbiota can be found in and on many multicellular organisms (all multicellular organisms studied to date from plants to animals), microbiota found in and on human are preferred. Such microbiota are referred to herein as “human microbiota” (wherein the term human refers specifically to the localization/residence of the microbiota). Within the context of the present invention, the microbiota sequence variant is a human microbiota sequence variant.

The term “immunogenic compound” refers to a compound comprising an antigenic peptide according to the present invention. An “immunogenic compound” is able to induce/elicit, increase, prolong or maintain an immune response against said antigenic peptide in a subject to whom it is administered. In some embodiments, immunogenic compounds comprise at least one antigenic peptide, or alternatively at least one compound comprising such an antigenic peptide, linked to a protein, such as a carrier protein.

A “carrier protein” is usually a protein, which is able to transport a cargo, such as the antigenic peptide according to the present invention. For example, the carrier protein may transport its cargo across a membrane. In the context of the present invention, a carrier protein in particular (also) encompasses a peptide or a polypeptide that is able to elicit an immune response against the antigenic peptide that is linked thereto. Carrier proteins are known in the art.

Alternatively, such carrier peptide or polypeptide may be co-administered in the form of immune adjuvant.

Preferably, the antigenic peptide as described herein may be co-administrated or linked, for example by covalent or non-covalent bond, to a protein/peptide having immuno-adjuvant properties, such as providing stimulation of CD4+Th1 cells. While the antigenic peptide as described herein preferably binds to MHC class I, CD4+ helper epitopes may be additionally used to provide an efficient immune response. Th1 helper cells are able to sustain efficient dendritic cell (DC) activation and specific CTL activation by secreting interferon-gamma (IFN-γ), tumor necrosis factor-alpha (TNF-α) and interleukin-2 (IL-2) and enhancing expression of costimulatory signal on DCs and T cells (Galaine et al., Interest of Tumor-Specific CD4 T Helper 1 Cells for Therapeutic Anticancer Vaccine. Vaccines (Basel). 2015 Jun. 30; 3(3):490-502).

For example, the adjuvant peptide/protein may preferably be distinct from the antigenic peptide according to the present invention. Preferably, the adjuvant peptide/protein is capable of recalling immune memory or provides a non-specific help or could be a specific helper peptide. Several helper peptides have been described in the literature for providing a nonspecific T cell help, such as tetanus helper peptide, keyhole limpet hemocyanin peptide or PADRE peptide (Adotévi et al., Targeting antitumor CD4 helper T cells with universal tumor-reactive helper peptides derived from telomerase for cancer vaccine. Hum Vaccin Immunother. 2013 May; 9(5):1073-7, Slingluff C L, The present and future of peptide vaccines for cancer: single or multiple, long or short, alone or in combination?Cancer J. 2011 September-October; 17(5):343-50). Accordingly, tetanus helper peptide, keyhole limpet hemocyanin peptide and PADRE peptide are preferred examples of such adjuvant peptide/proteins. This peptide represents another example of a helper peptide (having immuno-adjuvant properties), which is preferred in the context of the present invention. Another preferred example is h-pAg T13L (Bhasin M, Singh H, Raghava GP (2003) MHCBN: a comprehensive database of MHC binding and non-binding peptides. Bioinformatics 19: 665-666). Further examples of preferred helper peptides include the UCP2 peptide (for example as described in WO 2013/135553 A1 or in Dosset M, Godet Y, Vauchy C, Beziaud L, Lone Y C, Sedlik C, Liard C, Levionnois E, Clerc B, Sandoval F, Daguindau E, Wain-Hobson S, Tartour E, Langlade-Demoyen P, Borg C, Adotévi O: Universal cancer peptide-based therapeutic vaccine breaks tolerance against telomerase and eradicates established tumor. Clin Cancer Res. 2012 Nov. 15; 18(22):6284-95. doi: 10.1158/1078-0432.CCR-12-0896. Epub 2012 Oct. 2) and the BIRC5 peptide (for example as described in EP2119726 A1 or in Widenmeyer M, Griesemann H, Stevanović S, Feyerabend S, Klein R, Attig S, Hennenlotter J, Wernet D, Kuprash D V, Sazykin A Y, Pascolo S, Stenzl A, Gouttefangeas C, Rammensee HG: Promiscuous survivin peptide induces robust CD4+ T-cell responses in the majority of vaccinated cancer patients. Int J Cancer. 2012 Jul. 1; 131(1):140-9. doi: 10.1002/ijc.26365. Epub 2011 Sep. 14). The most preferred helper peptide is the UCP2 peptide (amino acid sequence: KSVWSKLQSIGIRQH; SEQ ID NO: 39, for example as described in WO 2013/135553 A1 or in Dosset et al., Clin Cancer Res. 2012 Nov. 15; 18(22):6284-95.

As used herein, the term “immunogenic composition” refers to a composition that is able to elicit, induce, increase, prolong or maintain an immune response, in particular which elicits, induces, increases, prolongs or maintains an immune response, when it is administered to a mammal, and especially when it is administered to a human individual. Preferably, an immunogenic composition further comprises one or more immuno-adjuvant substances.

By “pharmaceutically acceptable excipient or carrier”, it is meant herein a compound of pharmaceutical grade which improves the delivery, stability or bioavailability of an active agent, and can be metabolized by, and is non-toxic to, a subject to whom it is administered. Preferred excipients and carriers according to the invention include any of the excipients or carriers commonly used in pharmaceutical products, such as, for example, water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable excipients or carriers may further comprise minor amounts of auxiliary substances such as wetting or emulsifying agents, or preservatives.

By “vaccine”, it is meant herein a composition capable of stimulating the immune system of a living organism so that protection against a harmful antigen is provided, either through prophylaxis or through therapy. Prophylactic vaccines are preferred. Preferably, a vaccine or a vaccine composition further comprises one or more immuno-adjuvant substances.

According to the different aspects and embodiments of the invention described herein, a “subject” or “host” preferably refers to a mammal, and most preferably to a human being. Said subject may have, been suspected of having, or be at risk of developing cancer.

The term “cancer”, as used herein, refers to a malignant neoplasm. In particular, the term “cancer” refers herein to any member of a class of diseases or disorders that are characterized by uncontrolled division of cells and the ability of these cells to invade other tissues, either by direct growth into adjacent tissue through invasion or by implantation into distant sites by metastasis. Metastasis is defined as the stage in which cancer cells are transported through the bloodstream or lymphatic system. It encompasses, among others, esophageal cancer, gastric cancer, duodenal cancer, small intestinal cancer, appendiceal cancer, large bowel cancer, colon cancer, rectum cancer, colorectal cancer, anal cancer, pancreatic cancer, liver cancer, gallbladder cancer, spleen cancer, renal cancer, bladder cancer, prostatic cancer, testicular cancer, uterine cancer, endometrial cancer, ovarian cancer, vaginal cancer, vulvar cancer, breast cancer, pulmonary cancer, thyroid cancer, thymus cancer, brain cancer, nervous system cancer, gliomas, oral cavity cancer, skin cancer, blood cancer, lymphomas, eye cancer, bone cancer, bone marrow cancer, muscle cancer, etc. . . . . In the context of the present invention, melanoma, head and neck, breast, colorectal or renal cancer (such as clear cell renal cell carcinoma) are preferred.

As used herein, the terms “preventing”, “prevention”, “prophylaxis” or “prevent” generally mean to avoid or minimize the onset or development of a disease or condition before its onset, while the terms “treating, “treatment” or “treat” encompass reducing, ameliorating or curing a disease or condition (or symptoms of a disease or condition) after its onset. The term “preventing” encompasses “reducing the likelihood of occurrence of” or “reducing the likelihood of reoccurrence”.

An “effective amount” or “effective dose” as used herein is an amount which provides the desired effect. For therapeutic purposes, an effective amount is an amount sufficient to provide a beneficial or desired clinical result. The preferred effective amount for a given application can be easily determined by the skilled person taking into consideration, for example, the size, age, weight of the subject, the type of disease/disorder to be prevented or treated, and the amount of time since the disease/disorder began. In the context of the present invention, in terms of prevention or treatment, an effective amount of the composition is an amount that is sufficient to induce a humoral and/or cell-mediated immune response directed against the disease/disorder.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

Additional definitions are provided throughout the specification.

The present invention may be understood more readily by reference to the following detailed description, including preferred embodiments of the invention, and examples included herein.

### DETAILED DESCRIPTION

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Antigenic Peptides According to the Present Invention

In a first aspect, the present invention provides antigenic peptide comprising or consisting of an amino acid sequence as set forth in any one of SEQ ID NOs 1-16 and SEQ ID NOs 40-42.

The present invention also provides an antigenic peptide comprising or consisting of an amino acid sequence as set forth in any one of SEQ ID NOs 1-16 and SEQ ID NOs 40-42 wherein, optionally, one or two amino acid residues may be substituted, deleted or added.

The present inventors have identified a set of antigenic peptides that can be used to induce a specific immune response against tumor cells. Those antigenic peptides are distinct from, but have amino acid similarity to, (fragments of) human tumor antigens, as shown in Table 1 below. In particular, the antigenic peptides according to the present invention are comprised in polypeptides and proteins produced by commensal bacteria from the human gut. Accordingly, the antigenic peptides according to the present invention are not human sequences, but bacterial sequences. Without wishing to be bound by any particular theory, the inventors believe that the human immune repertoire contains T-cell clones that are reactive against bacterial peptides (comprised in proteins produced by commensal bacteria from the gut), which have amino acid similarity to fragments of human tumor antigens. In particular, the antigenic peptides according to the present invention can elicit a stronger immune response than the corresponding human peptides, since T cells able to recognize strictly human peptides have been depleted as recognizing self-antigens during maturation, which is not the case for the antigenic peptides according to the present invention. This may explain why the antigenic peptides described herein are able to induce an immune response, and especially a T-cell response, when these peptides are administered to a (human) individual. Accordingly, without being bound to any theory the inventors assume that proteins produced by commensal bacteria from the gut are able to “mimic” tumor antigens, and can be used for triggering a specific immune response against tumor cells. These findings provide further evidence that commensal bacteria may contribute to tumor cells eradication.

Accordingly, the invention relates to antigenic peptides having amino acid similarity with a tumor antigen. The expression “having amino acid similarity with a tumor antigen” as used herein, refers in particular to a sequence variant of a fragment (epitope) of a (reference) human tumor antigen, such as CDC20 or the other exemplified human tumor antigens described below in Table 1.

A “sequence variant” typically shares, in particular over the whole length of the sequence, at least 50% sequence identity with a reference sequence, such as a fragment of a (reference) tumor antigen. Preferably, the sequence variant shares at least 70% or 75%, preferably at least 80% or 85%, more preferably at least 90%, even more preferably at least 95%, still more preferably at least 96% or 97%, and particularly preferably at least 98% or 99% sequence identity with the reference sequence, such as a fragment of a (reference) tumor antigen. Sequence identity may be calculated as known in the art, in particular as described below. Preferably, a sequence variant preserves the specific function of the reference sequence, for example its function as tumor epitope and/or its ability to elicit or maintain an immune response. In particular, an amino acid sequence variant has an altered sequence in which one or more of the amino acids in the reference sequence is mutated, e.g. deleted or substituted, or one or more amino acids are inserted into the sequence of the reference amino acid sequence. For example, variant sequences which are at least 90% identical have no more than 10 alterations, i.e. any combination of deletions, insertions or substitutions, per 100 amino acids of the reference sequence.

Methods for comparing the identity (similarity) of two or more sequences are well known in the art. The percentage to which two sequences are identical can, e.g., be determined using a mathematical algorithm. A preferred, but not limiting, example of a mathematical algorithm which can be used is the algorithm of Karlin et at (1993), PNAS USA, 90:5873-5877. Such an algorithm is integrated in the BLAST family of programs, e.g. BLAST or NBLAST program (see also Altschul et al., 1990, J. Mol. Biol. 215, 403-410 or Altschul et al. (1997), Nucleic Acids Res, 25:3389-3402), accessible through the home page of the NCBI at world wide web site ncbi.nlm.nih.gov) and FASTA (Pearson (1990), Methods Enzymol. 183, 63-98; Pearson and Lipman (1988), Proc. Natl. Acad. Sci. U.S.A 85, 2444-2448). Sequences which are identical to other sequences to a certain extent can be identified by these programmes.

Furthermore, programs available in the Wisconsin Sequence Analysis Package, version 9.1 (Devereux et al., 1984, Nucleic Acids Res., 387-395), for example the programs BESTFIT and GAP, may also be used to determine the % identity between two polynucleotides and the % identity between two (poly)peptide sequences. BESTFIT uses the “local homology” algorithm of Smith and Waterman (1981), J. Mol. Biol. 147, 195-197 and finds the best single region of similarity between two sequences.

In particular, the antigenic peptides according to the present invention have a core sequence identical with the core sequence of the sequence of the epitope (fragment) of the (human) reference tumor antigen. Moreover, the core sequence displays a high prevalence based on the frequency of the proteins present in the human microbiota where the core sequence is found.

Therefore, the core sequence represents a major feature of the antigenic peptides according to the present invention. The inventors have thus identified core sequences of highly interest with high prevalence since they are present in several sequence variants of a fragment of a (reference) tumor antigen and/or in several human microbiota proteins with high frequency in a significant portion of the general human population. Preferably, in the antigenic peptide comprising or consisting of an amino acid sequence as set forth in any one of SEQ ID NOs 1-16 and SEQ ID NOs 40-42 wherein, optionally, one or two amino acid residues may be substituted, deleted or added, the core sequence is maintained.

Preferably, the antigenic peptide according to the present invention comprises or consists of an amino acid sequence according to any one of SEQ ID NOs 1 to 16 and SEQ ID NOs 40-42. In some embodiments, the antigenic peptide consists of or consists essentially of an amino acid sequence according to any one of SEQ ID NOs 1 to 16 and SEQ ID NOs 40-42.

In some embodiments, the antigenic peptide may be modified and/or it may include non-peptide bonds (e.g. as described above). For example, the antigenic peptide may be modified at its N-terminus and/or at its C-terminus (e.g., to be labelled or to be linked to a carrier or substrate). Such modifications usually depend on the intended purpose and are well-known in the art.

The antigenic peptides disclosed herein can be prepared using well known techniques. For example, the peptides can be prepared synthetically, by recombinant DNA technology or chemical synthesis. Peptides disclosed herein can be synthesized individually or as longer polypeptides comprising two or more peptides (e.g., two or more peptides or a peptide and a non-peptide). The antigenic peptides can be isolated i.e., purified to be substantially free of other naturally occurring host cell proteins and fragments thereof, e.g., at least about 70%, 80% or 90% purified. Preferably, the antigenic peptides according to the present invention are isolated antigenic peptides.

In some embodiments, the antigenic peptides according to the present invention have the ability to bind to a molecule of the human major histocompatibility complex (MHC, such as MHC class-I (MHC I) molecules; or—in an elongated form, such as a length-variant—MHC class-II (MHC II) molecules. Preferably, the antigenic peptide according to the present invention can bind to MHC class I (major histocompatibility complex class I, MHC I) molecules.

MHC class I molecules present epitopes to killer T cells, also called cytotoxic T lymphocytes (CTLs). A CTL expresses CD8 receptors, in addition to TCRs (T-cell receptors). When a CTL's CD8 receptor docks to a MHC class I molecule, if the CTL's TCR fits the epitope within the MHC class I molecule, the CTL triggers the cell to undergo programmed cell death by apoptosis. This route is particularly useful in prevention and/or treatment of cancer, since cancer cells are directly attacked. In humans there are three different genetic loci that encode MHC class I molecules (the MHC-molecules of the human are also designated human leukocyte antigens (HLA)): HLA-A, HLA-B, and HLA-C. Accordingly, MHC class I comprises HLA-A, HLA-B, and HLA-C molecules in humans. HLA-A*01, HLA-A*02, HLA-A*24 and HLA-1B*07 are examples of different MHC class I alleles that can be expressed from these loci.

For instance, the antigenic peptide according to the invention may bind to HLA-A*01, HLA-A*02, HLA-A*24 and HLA-B*07 molecules. In some embodiments, the antigenic peptide according to the invention binds to HLA-A*02. Typically, peptides (epitopes) having a length of 8-11, amino acids are presented by MHC I.

In general, the antigenic peptide according to the present invention may be of any length. Preferably, the length of the antigenic peptide according to the present invention does not exceed 350 amino acids. For example, the maximum length of the antigenic peptide according to the present invention may be 300 or 250 amino acids. More preferably, the maximum length of the antigenic peptide according to the present invention does not exceed 200 amino acids, e.g., not more than 190, 180, 170, 160, 150, 140, 130, 120, 110, 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14 or 13 amino acids. In particular, the length of the antigenic peptides according to the present invention is preferably at most 30 or 25 amino acids, more preferably at most 20 or 15 amino acids, with smaller peptides of 8 to 15 amino acids or of 8-11 amino acids (such as 8, 9, 10 or 11 amino acids) in length being even more preferred; and peptides having a length of 9 or 10 amino acids being still more preferred. In particular, the antigenic peptides are not the full-length proteins produced by commensal bacteria from the gut from which the antigenic peptides are derived from. In other words, the antigenic peptide of the invention is preferably a fragment of a full-length protein (produced by human microbiota).

Similarly, the “fragment/epitope” of the (reference) tumor antigen, which typically serves as reference sequence, preferably comprises consecutive 8 to 11 amino acids, preferably 9 or 10 amino acids, of the tumor antigen. It is understood that the “fragment/epitope” of the (reference) tumor antigen is not the full-length tumor antigen (protein).

A “fragment” (of a protein or nucleic acid (sequence)), as used herein, has preferably a maximum length of 95%, 90%, 85%, 80%, 75%, 70%, 65%, 60%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% of the full-length (reference) protein/nucleic acid/sequence.

In some embodiments, the length of the fragment does not exceed 50% of the length of the (full-length) (reference) protein/nucleic acid. In other embodiments, the length of the fragment of the (reference) protein/nucleic acid does not exceed 20% or 10% of the length of the (full-length) (reference) protein/nucleic acid.

In more general, the present invention provides an antigenic peptide, which comprises or consists of a microbiota sequence variant of a fragment of a human tumor antigen. The human tumor antigen may be selected from the group consisting of CDC20, KIF2C, UBE2C, ANKRD30A, AURKA, CDH17, CEACAM5, MMP11, OR51 E2 and TOP2A. The fragment/epitope of the human (reference) tumor antigen may be selected from the group consisting of any one of SEQ ID NOs 17-31 and 38. In some embodiments, the antigenic peptide comprises or consists of a microbiota variant of a human reference peptide according to any one of SEQ ID NOs 17-31 and 38.

In some embodiments, the antigenic peptide induces T-cell cross-reactivity against the human epitope of a (reference) tumor antigen. T-cell cross-reactivity is a phenomenon of the immune system defined as the recognition of two or more peptide-MHC complexes (pMHCs) by the T-cell receptor (TCR).

Epitope mimicry relates to the concept of sequence and structure similarity between foreign antigens and self-antigens as a trigger mechanism to elicit a cross reactive immune response against the self-antigens. Interestingly, such epitope mimicry offers a possible way to bypass the repertoire restriction of human T cells due to clonal depletion of T cells recognizing self-antigens.

In particular, antigens (i.e. the antigenic peptides according to the present invention) distinct from self-antigens (e.g. human epitope of a tumor antigen), but sharing sequence similarity with the self-antigen, (i) can still be recognized due to the cross-reactivity of the T-cell receptor and (ii) it is expected that such antigens are recognized by T cell/TCR that have not been depleted during T cell education process. Accordingly, such antigens are able to elicit a strong immune response leading to clonal expansion of T cell harboring potential cross reactivity with self-antigens.

T cell receptor cross-reactivity with the epitope of a human (reference) tumor antigen may be measured as shown in the section EXAMPLES by ELISPOT-IFNγ assay. Briefly HLA-A2 transgenic mice (e.g. HHD DR1 mice expressing human HLA-A2 and HLA-DR1 MHC and lacking the murine H-2 class I and class II MHCs and/or HHD DR3 mice expressing human HLA-A2 and HLA-DR3 MHC) may be immunized on day 0 (d0) with a prime injection, and later, e.g. on d14, with a boost injection with an antigenic peptide of the present invention or, in a control group, with the corresponding human (reference) peptide. Thereafter, e.g. seven days after the boost injection (i.e. on d21), mice may be sacrificed and the splenocytes may be stimulated in vitro with the antigenic peptide of the present invention to assess their capacity to secrete IFN-gamma as assessed by ELISPOT.

Table 1 below provides an overview over the antigenic peptides according to the present invention with their amino acid sequences and SEQ ID NOs. Table 1 also provides information to which tumor antigen (also referred to herein as “human reference peptide”) each antigenic peptide according to the present invention relates. SEQ ID NOs 1 to 16 and SEQ ID NOs 40-42 refer to HLA-A*02 antigenic peptides according to the present invention.

TABLE 1
HLA-A*02 Antigenic peptides according
to the invention.

| | | SEQ ID | | |
|---|---|---|---|---|
| | | NO. | | SEQ ID |
| | | human | | NO. |
| | Sequence | refer- | | anti- |
| | human | ence | Sequence | genic |
| Tumor | reference | pep- | antigenic | pep- |
| antigen | peptide | tide | peptide | tide |
| CDC20 | SLPDRILDA | 17 | SLPDRILTV | 1 |
| KIF2C | AINPELLQL | 18 | ALNPELLAL | 2 |
| KIF2C | AINPELLQL | 18 | QMNPELLTL | 40 |
| KIF2C | AINPELLQL | 18 | VMNPELLLL | 41 |
| KIF2C | AINPELLQL | 18 | VLNPELLML | 42 |
| UBE2C | ALYDVRTIL | 19 | FLYDVRTYL | 3 |
| UBE2C | ALYDVRTIL | 19 | YLYDVRTAL | 4 |
| UBE2C | ILLSIQSLL | 20 | SLLSIQSYV | 5 |
| UBE2C | RLQQELMTL | 21 | YLQQELMNL | 6 |
| ANKRD30A | AVYSEILSV | 22 | ALYSEILTV | 7 |
| ANKRD30A | KILDTVHSC | 23 | LILDTVHSL | 8 |
| ANKRD30A | SLDQKLFQL | 24 | TLDQKLFMV | 9 |
| AURKA | YLILEYAPL | 25 | YLILEYATV | 10 |
| CDH17 | LVIGIILAV | 26 | KIIGIILAV | 11 |
| CEACAM5 | YLSGANLNL | 27 | YLSGANLFV | 12 |
| MMP11 | KVWSDVTPL | 28 | IVWSDVTYV | 13 |
| OR51E2 | AQIGIVAVV | 29 | AVIGIVAAV | 14 |
| TOP2A | ILNSTTIEI | 30 | LLNSTTIPI | 15 |
| TOP2A | ALIFGQLLT | 31 | ALIFGQLLL | 16 |

In some embodiments, the antigenic peptide according to the present invention is a sequence variant of a fragment of the tumor antigen CDC20 (human reference peptide), such as “SLPDRILDA” (SEQ ID NO: 17). Preferably, the antigenic peptide according to the present invention is a sequence variant of a fragment of the tumor antigen CDC20, such as the antigenic peptide comprising or consisting of an amino acid sequence as set forth in SEQ ID NO: 1.

In some embodiments, the antigenic peptide according to the present invention is a sequence variant of a fragment of the tumor antigen KIF2C (human reference peptide), such as “AINPELLQL” (SEQ ID NO: 18). Preferably, the antigenic peptide according to the present invention is a sequence variant of a fragment of the tumor antigen KIF2C, such as an antigenic peptide comprising or consisting of an amino acid sequence as set forth in SEQ ID NO: 2 and in SEQ ID NO: 40-42.

In some embodiments, the antigenic peptide according to the present invention is a sequence variant of a fragment of the tumor antigen UBE2C (human reference peptide), such as “ALYDVRTIL” (SEQ ID NO: 19), “ALYDVRTILL” (SEQ ID NO: 38), “ILLSIQSLL” (SEQ ID NO: 20) or “RLQQELMTL” (SEQ ID NO: 21). More preferably, the antigenic peptide according to the present invention is a sequence variant of the UBE2C fragment (human reference peptide) “ALYDVRTIL” (SEQ ID NO: 19), such as an antigenic peptide comprising or consisting of an amino acid sequence as set forth in SEQ ID NO: 3-4. It is also more preferred that the antigenic peptide according to the present invention is a sequence variant of the UBE2C fragment (human reference peptide) “ILLSIQSLL” (SEQ ID NO: 20), such as an antigenic peptide comprising or consisting of an amino acid sequence as set forth in SEQ ID NO: 5. It is also more preferred that the antigenic peptide according to the present invention is a sequence variant of the UBE2C fragment (human reference peptide) “RLQQELMTL” (SEQ ID NO: 21), such as an antigenic peptide comprising or consisting of an amino acid sequence as set forth in SEQ ID NO: 6.

In some embodiments, the antigenic peptide according to the present invention is a sequence variant of a fragment of the tumor antigen ANKRD30A (human reference peptide), such as “AVYSEILSV” (SEQ ID NO: 22), “KILDTVHSC” (SEQ ID NO: 23) or “SLDQKLFQL” (SEQ ID NO: 24). More preferably, the antigenic peptide according to the present invention is a sequence variant of the ANKRD30A fragment (human reference peptide) “AVYSEILSV” (SEQ ID NO: 22), such as an antigenic peptide comprising or consisting of an amino acid sequence as set forth in SEQ ID NO: 7. It is also more preferred that the antigenic peptide according to the present invention is a sequence variant of the ANKRD30A fragment (human reference peptide) “KILDTVHSC” (SEQ ID NO: 23), such as an antigenic peptide comprising or consisting of an amino acid sequence as set forth in SEQ ID NO: 8. It is also more preferred that the antigenic peptide according to the present invention is a sequence variant of the ANKRD30A fragment (human reference peptide) “SLDQKLFQL” (SEQ ID NO: 24), such as an antigenic peptide comprising or consisting of an amino acid sequence as set forth in SEQ ID NO: 9.

In some embodiments, the antigenic peptide according to the present invention is a sequence variant of a fragment of the tumor antigen AURKA (human reference peptide), such as “YLILEYAPL” (SEQ ID NO: 25). Preferably, the antigenic peptide according to the present invention is a sequence variant of a fragment of the tumor antigen AURKA, such as an antigenic peptide comprising or consisting of an amino acid sequence as set forth in SEQ ID NO: 10.

In some embodiments, the antigenic peptide according to the present invention is a sequence variant of a fragment of the tumor antigen CDH17 (human reference peptide), such as “LVIGIILAV” (SEQ ID NO: 26). Preferably, the antigenic peptide according to the present invention is a sequence variant of a fragment of the tumor antigen CDH17, such as an antigenic peptide comprising or consisting of an amino acid sequence as set forth in SEQ ID NO: 11.

In some embodiments, the antigenic peptide according to the present invention is a sequence variant of a fragment of the tumor antigen CEACAM5 (human reference peptide), such as “YLSGANLNL” (SEQ ID NO: 27). Preferably, the antigenic peptide according to the present invention is a sequence variant of a fragment of the tumor antigen CEACAM5, such as an antigenic peptide comprising or consisting of an amino acid sequence as set forth in SEQ ID NO: 12.

In some embodiments, the antigenic peptide according to the present invention is a sequence variant of a fragment of the tumor antigen MMP11 (human reference peptide), such as “KVWSDVTPL” (SEQ ID NO: 28). Preferably, the antigenic peptide according to the present invention is a sequence variant of a fragment of the tumor antigen MMP11, such as an antigenic peptide comprising or consisting of an amino acid sequence as set forth in SEQ ID NO: 13.

In some embodiments, the antigenic peptide according to the present invention is a sequence variant of a fragment of the tumor antigen OR51E2 (human reference peptide), such as “AQIGIVAVV” (SEQ ID NO: 29). Preferably, the antigenic peptide according to the present invention is a sequence variant of a fragment of the tumor antigen OR51E2, such as an antigenic peptide comprising or consisting of an amino acid sequence as set forth in SEQ ID NO: 14.

In some embodiments, the antigenic peptide according to the present invention is a sequence variant of a fragment of the tumor antigen TOP2A (human reference peptide), such as “ILNSTTIEI” (SEQ ID NO: 30) or “ALIFGQLLT” (SEQ ID NO: 31). More preferably, the antigenic peptide according to the present invention is a sequence variant of the TOP2A fragment (human reference peptide) “ILNSTTIEI” (SEQ ID NO: 30), such as an antigenic peptide comprising or consisting of an amino acid sequence as set forth in SEQ ID NO: 15.

It is also more preferred that the antigenic peptide according to the present invention is a sequence variant of the TOP2A fragment (human reference peptide) “ALIFGQLLT” (SEQ ID NO: 31), such as an antigenic peptide comprising or consisting of an amino acid sequence as set forth in SEQ ID NO: 16.

Preferably, the antigenic peptide according to the present invention comprises or consists of an amino acid sequence as set forth in any one of SEQ ID NOs 1, 2 and 3. In some embodiments, the antigenic peptide comprises or consists of an amino acid sequence as set forth in SEQ ID NO: 1. In some embodiments, the antigenic peptide comprises or consists of an amino acid sequence as set forth in SEQ ID NO: 2. In some embodiments, the antigenic peptide comprises or consists of an amino acid sequence as set forth in SEQ ID NO: 3.

As shown in the examples herein, the specific antigenic peptides according to the present invention allow the raise of a strong immune response against themselves, and most importantly, allow the raise of a strong immune response against peptides having amino acid similarity therewith which are comprised in the tumor antigen, even if the human reference peptides comprised in the tumor antigen may be tolerogenic.

Advantageously, the antigenic peptides according to the present invention may be in the form of immunogenic compounds, in particular for use in the prevention or in the treatment of cancer.

### Immunogenic Compounds Comprising the Antigenic Peptide According the Invention

In a further aspect, the present invention also provides an immunogenic compound comprising an antigenic peptide according to the present invention as described above. In particular, preferred embodiments of the antigenic peptide as described above also apply for the immunogenic compound according to the present invention.

In general, the term “immunogenic compound” includes all kinds of compounds comprising the antigenic peptide according to the present invention. For example, the antigenic peptide according to the present invention may be linked to a carrier molecule or the antigenic peptide according to the present invention may be comprised in a polypeptide or protein (which polypeptide or protein may occur “separately”, i.e. not linked to any other compound, or the polypeptide or protein comprising the antigenic peptide may be linked to a carrier molecule).

The types of carrier molecules used for generating an immunogenic compound of the invention, such as an immunogenic compound comprising or consisting of a polypeptide of formula (I) linked to a carrier molecule, are well in the general knowledge of the one skilled in the art. In particular, the function of the carrier molecule may be to provide cytokine help (or T-cell help) in order to enhance the immune response against tumor antigen.

Preferably, the immunogenic compound according to present invention comprises the antigenic peptide and a carrier molecule, in particular wherein the antigenic peptide (or a polypeptide or protein comprising the antigenic peptide) is linked to a carrier molecule. A preferred carrier molecule is a carrier protein or a carrier peptide. According to a preferred embodiment, the antigenic peptide as above defined, or a polypeptide/protein comprising said antigenic peptide, is linked to a carrier protein or a carrier peptide, for example by a covalent or non-covalent bond. Alternatively, such a carrier protein or carrier peptide as described herein) may be (separately) co-administered in the form of immune adjuvant (i.e., not as an “immunogenic compound”, but as co-administration/combination therapy as described herein below).

In some embodiments, the antigenic peptide as described herein, or a polypeptide/protein comprising the antigenic peptide, may be co-administrated or linked, for example by covalent or non-covalent bond, to a protein/peptide having immuno-adjuvant properties, such as providing stimulation of CD4+Th1 cells. While the antigenic peptide as described herein preferably binds to MHC class I, CD4+ helper epitopes may be additionally used to provide an efficient immune response. Th1 helper cells are able to sustain efficient dendritic cell (DC) activation and specific CTL activation by secreting interferon-gamma (IFN-γ), tumor necrosis factor-alpha (TNF-α) and interleukin-2 (IL-2) and enhancing expression of costimulatory signal on DCs and T cells (Galaine et al., Interest of Tumor-Specific CD4 T Helper 1 Cells for Therapeutic Anticancer Vaccine. Vaccines (Basel). 2015 Jun. 30; 3(3):490-502). For example, the adjuvant peptide/protein may be a non-tumor antigen that recalls immune memory or provides a non-specific help or could be a specific tumor-derived helper peptide. Several helper peptides have been described in the literature for providing a nonspecific T cell help, such as tetanus helper peptide, keyhole limpet hemocyanin peptide or PADRE peptide (Adotévi et al., Targeting antitumor CD4 helper T cells with universal tumor-reactive helper peptides derived from telomerase for cancer vaccine. Hum Vaccin Immunother. 2013 May; 9(5):1073-7, Slingluff CL, The present and future of peptide vaccines for cancer: single or multiple, long or short, alone or in combination?Cancer J. 2011 September-October; 17(5):343-50). Accordingly, tetanus helper peptide, keyhole limpet hemocyanin peptide and PADRE peptide are examples of such adjuvant peptide/proteins. Moreover, the adjuvant peptide/protein may be a specific tumor derived helper peptide. Specific tumor derived helper peptides are typically presented by MHC class II, in particular by HLA-DR, HLA-DP or HLA-DQ. Specific tumor derived helper peptides may be fragments of sequences of shared overexpressed tumor antigens, such as HER2, NY-ESO-1, hTERT or IL13RA2. Such fragments have preferably a length of at least 10 amino acids, more preferably of at least 11 amino acids, even more preferably of at least 12 amino acids and most preferably of at least 13 amino acids. In particular, fragments of shared overexpressed tumor antigens, such as HER2, NY-ESO-1, hTERT, having a length of 13 to 24 amino acids are preferred. Preferred fragments bind to MHC class II and may, thus, be identified using, for example, the MHC class II binding prediction tools of IEDB (Immune epitope database and analysis resource; Supported by a contract from the National Institute of Allergy and Infectious Diseases, a component of the National Institutes of Health in the Department of Health and Human Services; URL: http://www.iedb.org/; http//tools.iedb.org/mhcii/). Preferably, the adjuvant peptide/protein is the UCP2 peptide (amino acid sequence: KSVWSKLQSIGIRQH; SEQ ID NO: 39, for example as described in WO 2013/135553 A1 or in Dosset et al., Clin Cancer Res. 2012 Nov. 15; 18(22):6284-95).

It is also preferred that the immunogenic compound according to the present invention is a polypeptide or a protein comprising the antigenic peptide according to the present invention. Preferably, such a protein or polypeptide is a recombinant protein or polypeptide, for example a fusion protein. The term “recombinant” means that it does not occur in nature. In some embodiments, the antigenic peptide according to the present invention may be part of a fusion protein, for example fused to the N-terminal amino acids of the HLA-DR antigen-associated invariant chain (li), or fused to (or into the sequence of) an antibody, such as, for example, an antibody that is specific for dendritic cells.

Preferably, the immunogenic compound according to the present invention comprises or consists of a polypeptide of formula (I)

PepNt-CORE-PepCt (I)
wherein:
   “PepNt” consists of a polypeptide having a length varying from 0 to 500 amino acid residues and is located at the N-terminal end of the polypeptide of formula (I);
   “CORE” consists of an antigenic peptide according to the present invention as defined above; and
   “PepCt” consists of a polypeptide having a length varying from 0 to 500 amino acid residues and is located at the C-terminal end of the polypeptide of formula (I).

For example, the immunogenic compound may comprise or consist of a polypeptide of formula (Ia) or (Ib):

PepNt-CORE (Ia); or

CORE-PepCt (Ib)
wherein “PepNt” and “PepCt” and “CORE” are as defined above.

Preferably, the polypeptide of formula (I), (Ia) or (Ib) is a fusion peptide or fusion protein, in particular a recombinant fusion peptide or protein.

It is also preferred that the polypeptide or the immunogenic compound as defined above, comprises from 9 to 1000 amino acids; which includes 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67?68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 250, 300, 350, 400, 450, 500, 600, 700, 800, 900 and 1000 amino acids. Accordingly, the length of “PepNt” and “PepCt”, if applicable, may be defined accordingly.

Thus, “PepNt” and “PepCt”, as defined above, may comprise from 0 to 500 amino acid residues; which includes 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 250, 300, 350, 400, 450, and 500 amino acid residues.

Preferably, the antigenic peptide is linked to a carrier molecule, in particular to a carrier protein, preferably by covalent or non-covalent bond. The carrier molecule to which the peptide is optionally bound can be selected from a wide variety of known carriers. Examples of carrier molecules for vaccine purposes encompass proteins such as human or bovine serum albumin and keyhole limpet haemocyanin (KLH) and fatty acids. Other embodiments of carrier molecules to which an antigenic peptide of formula (I) may be covalently linked include bacterial toxins or toxoids, such as diphtheria, cholera, E. coli heat labile or tetanus toxoids, the N. meningitidis outer membrane protein (European patent application n° EP0372501), synthetic peptides (European patent applications no EP0378881 and n° EP0427347), heat shock proteins (PCT application no WO93/17712), Pertussis proteins (PCT application no WO98/58668), protein D from H. influenzae (PCT application n° WO00/56360.) and toxin A or B from C. difficile (International patent application WO00/61761).

Moreover, in the polypeptide according to formula (I), (Ia) or (Ib), “PepNt” and/or “PepCt” may preferably correspond to such a protein/peptide having immuno-adjuvant properties, such as providing stimulation of CD4+Th1 cells as described herein.

In some embodiments, the antigenic peptide according to the present invention (or the polypeptide/protein comprising said antigenic peptide) is covalently bound to the carrier molecule through a linker moiety. For example, the linker agent may be selected from the group consisting of GMBS (N-[γ-maleimidobutyryl-oxy]succinimide ester), Sulfo-GMBS (N-[γ-maleimidobutyryl-oxyl]sulfosuccinimide ester), SMPB (succinimidyl 4-[p-maleimidophenyl]butyrate) and Sulfo-SMPB (sulfosuccinimidyl 4-[p-maleimidophenyl]butyrate).

### Peptide-MHC(pMHC)Mu/Timers Comprising the Antigenic Peptide

In a further aspect, the present invention also provides a peptide-MHC (pMHC) multimer comprising an antigenic peptide according to the present invention.

As used herein, the term “peptide-MHC multimer” (pMHC) refers to a stable multimeric complex composed of major histocompatibility complex (MHC) protein subunits loaded with an antigenic peptide of the invention. In general, “MHC multimers” are oligomeric forms of MHC molecules. The main function of an MHC molecule is to bind to an antigen. According to the invention, said antigen is the antigenic peptide according to the invention. Accordingly, a complex of MHC proteins “loaded” with the antigenic peptide of the invention typically means that the antigenic peptide of the invention is bound to one or more of the MHC proteins. The “peptide-MHC multimers” (pMHC) of the invention include, but are not limited to, a peptide-MHC dimer, trimer, tetramer, pentamer, hexamer, heptamer or octamer. MHC tetramers and pentamers are preferred. The term “Major Histocompatibility Complex” (MHC) is a generic designation meant to encompass the histo-compatibility antigen systems described in different species including the human leucocyte antigens (HLA). In humans there are three major different genetic loci that encode MHC class I molecules: HLA-A, HLA-B, and HLA-C. HLA-A*01, HLA-A*02, and HLA-A*11 are examples of different MHC class I alleles that can be expressed from these loci.

In one embodiment of the invention, the pMHC multimer is a peptide/MHC class I multimer. In another particular embodiment, the pMHC multimer is a HLA corresponding to MHC class I/peptide multimer. Accordingly, the pMHC multimer may be a HLA-peptide multimer selected from the group consisting of HLA-A-peptide multimer, HLA-B-peptide multimer, HLA-C-peptide multimer, HLA-E-peptide multimer, MICA-peptide multimer and MICB-peptide multimer. Methods for obtaining pHMC multimers are known in the art and described, for example, in WO96/26962 and WO01/18053, which are incorporated herein by reference.

In addition to the MHC molecule and the antigenic peptide of the invention, the pMHC may contain further components, such as a multimerization agent and/or a label (e.g., for visualization). Examples of labels include, but are not limited to, fluorescent labels, e.g. fluorescently labelled proteins, such as streptavidin. Fluorescent labels include allophycocyanin (APC), phycoerythrin (PE), R-phycoerythrin (R-PE) and fluorescein isothiocyanate (FITC). A preferred label is biotin.

In one embodiment of the invention, said pMHC multimer can be used to visualize T cell populations that are specific for the MHC class I peptide complex or a HLAs corresponding to MHC class I/peptide complex as described here above. For example, the pMHC multimer may be a multimer where the heavy chain of the MHC is biotinylated, which allows combination as a tetramer with streptavidine. Such pMHC tetramer has an increased avidity for the appropriate TCR-carrier T lymphocytes and can therefore be used to visualize reactive populations by immunofluorescence. In another embodiment of the invention, said pMHC multimer can be used for the detection and/or isolation by screening (in flow cytometry or by immunomagnetic screening) of T cell populations that are specific for a pMHC complex as described here above.

### Antigenic Peptide-Specific T Lymphocytes

In a further aspect, the present invention also provides a T lymphocyte specific for an antigenic peptide according to the invention, in particular a cytotoxic T lymphocyte (CTL) specific for an antigenic peptide according to the invention. The T lymphocyte, in particular the CTL, is preferably an activated (cytotoxic) T lymphocyte specific for an antigenic peptide according to the invention. “Specificity” of a T lymphocyte is preferably understood as a T lymphocyte binding to the antigenic peptide and, additionally, binding to the tumor antigen corresponding to the antigenic peptide. In this regard, the T lymphocyte, in particular a CTL, cross-reacts to both the antigenic peptide and its tumor antigen counterpart, typically exhibiting a high level of sequence identity or similarity with the antigenic peptide.

The present invention further provides a method for producing (cytotoxic) T lymphocytes specific for an antigenic peptide according to the invention, in particular activated (cytotoxic) T lymphocytes specific for an antigenic peptide according to the invention, the method comprising contacting in vitro a T lymphocyte, in particular a CTL, with an antigen-loaded human class I or II MHC molecule expressed on the surface of an antigen-presenting cell or an artificial construct mimicking an antigen-presenting cell, wherein said antigen is an antigenic peptide according to the invention. Preferred antigen-presenting cells include dendritic cells. An artificial construct mimicking an antigen-presenting cell may be, for instance, a peptide-MHC multimer according to the invention. The step of contacting the T lymphocyte, in particular the CTL, with the antigen-loaded human class I or II MHC molecule expressed on the surface of the antigen-presenting cell or the artificial construct mimicking an antigen-presenting cell may be carried out for a period of time sufficient to activate said T lymphocyte, in particular CTL, in an antigen-specific manner. Preferably, the antigenic peptide is a preferred antigenic peptide as described above, such as an antigenic peptide comprising or consisting of an amino acid sequence as set forth in any one of SEQ ID NOs 1 to 16 and SEQ ID NOs 40 to 42, more preferably in any one of SEQ ID NOs 1, 2 and 3.

The present invention further relates to activated T cells, produced by the method according to the present invention, wherein said T cell selectively recognize a cell which expresses a polypeptide comprising the antigenic peptide according to the present invention and/or a polypeptide comprising the respective human reference peptide. In one particular embodiment, said T cell recognizes a cell which expresses a polypeptide comprising the antigenic peptide according to the present invention and a polypeptide comprising the respective human reference peptide, especially a tumor cell overexpressing this respective TAA as described herein.

The (activated) T cells that are directed against the antigenic peptides of the invention are useful in therapy. In particular, activated T cells, which are produced by the above method, selectively recognize a cell that aberrantly expresses a polypeptide that comprises an amino acid sequence of SEQ ID NO: 17-31 and 38 (i.e., a tumor antigen), for example, a polypeptide that comprises an amino acid sequence of as set forth in any one of SEQ ID NOs 17, 18, 19 and 38. In a particular embodiment, a cell that aberrantly expresses a polypeptide that comprises an amino acid sequence of SEQ ID NO: 17-31 and 38, is a tumor cell involved in the cancer to be treated.

Preferably, the T lymphocytes according to the present invention, which are specific for an antigenic peptide of the invention, may have (exhibit/express) memory markers. Such memory markers are preferably memory markers of gut memory cells, such as CCR9, CXCR3, CD103, CX3CR1 and α4β7+.

The T lymphocytes according to the present invention, which are specific for an antigenic peptide of the invention, are preferably more/stronger amplified after vaccination with antigenic peptide of the invention (derived from human microbiota sequences) as compared to vaccination with peptides not derived from microbiota sequences, such as the human (reference) sequence and/or a synthetic peptide (e.g., including mutations, which were, e.g., artificially introduced). In other words, vaccination of subjects with the antigenic peptide of the invention preferably increases the number of T lymphocytes according to the present invention, which are specific for said antigenic peptide of the invention, more than vaccination with respective human peptides or synthetic peptides (not derived from microbiota), which relate to the same reference epitope.

The T lymphocytes according to the present invention, which are specific for an antigenic peptide of the invention, are preferably more/stronger and/or faster amplified after vaccination in subjects having said peptide in the gut (expressed by the subject's microbiota), e.g., the peptide can be found in a stool sample of the subject, as compared to subjects not having said peptide in the gut (not expressed by the subject's microbiota), e.g. subjects where said peptide is not detectable in stool samples. In particular, subjects having said peptide in the gut (expressed by the subject's microbiota), may respond faster (faster T cell expansion) and/or have T cells from the desired type Tc1.

Cells Loaded with the Antigenic Peptide or the Immunogenic Compound

In a further aspect, the present invention also provides a cell loaded with an antigenic peptide according to the present invention or with the immunogenic compound comprising an antigenic peptide according to the present invention as described above. In particular, preferred embodiments of the antigenic peptide as described above also apply for such a cell according to the present invention.

A preferred cell loaded with the antigenic peptide according to the present invention or with the immunogenic compound according to the present invention is an antigen presenting cell (APC), more preferably a dendritic cell (DC).

APCs are of particular interest, as their main function is to process antigens and present it on the cell surface to the T cells of the immune system, so as to initiate and modulate T-cell responses in vivo. In the context of the present invention, it is preferred that the APCs are loaded with the antigenic peptide(s) and/or immunogenic compound(s) according to the invention. This may be done by exposing APCs in vitro with said antigenic peptide(s) and/or immunogenic compound(s) (as described in Rizzo MM, Alaniz L, Mazzolini G. Ex vivo loading of autologous dendritic cells with tumor antigens. Methods Mol Biol. 2014; 1139:41-4; Rolinski J, Hus I. Breaking immunotolerance of tumors: a new perspective for dendritic cell therapy. J Immunotoxicol. 2014 October; 11(4):311-8).

Preferred APCs according to the invention are dendritic cells (DCs). It can indeed be advantageous to combine at least one antigenic peptide or immunogenic compound according to the invention with DCs, as those are the most potent APCs and have been reported to be frequently functionally defective in cancer patients. DCs can be easily obtained by the skilled person in the art from either healthy compatible donors (i.e. the DCs are HLA-related) or from the patient himself provided that they are functional (i.e. the DCs are autologous), for example by direct isolation from the peripheral blood, or by derivation from peripheral blood cells such as CD14+ monocytes or CD34+ hematopoietic precursors (Figdor C G, de Vries I J, Lesterhuis W J, Melief C. Dendritic cell immunotherapy: mapping the way. Nat Med. 2004 May; 10(5):475-80). DCs can indeed be distinguished from other cells of peripheral blood by their surface markers, such as S100, p55, CD83, and/or OX62, and may thus be isolated and purified based on said markers using cell cultures techniques well-known in the art.

The present invention further relates to a method for preparing the cell loaded with the antigenic peptide according to the present invention, wherein the antigenic peptide is loaded onto class I or II MHC molecules expressed on the surface of the cell, in particular an antigen-presenting cell (or an artificial antigen-presenting cell) comprising a step of contacting (a sufficient amount of) the antigenic peptide with the cell, in particular the antigen-presenting cell.

### Nucleic Acids Encoding the Antigenic Peptides and Host Cells Comprising Nucleic Acids

In a further aspect, the present invention also provides a nucleic acid encoding the antigenic peptide according to the present invention, the polypeptide of formula (I) as defined above, or the immunogenic compound according to the present invention, wherein the immunogenic compound is a peptide or a protein. In particular, preferred embodiments of the antigenic peptide as described above also apply for such a nucleic acid according to the present invention. For example, antigenic peptides according to the present invention comprising or consisting of an amino acid sequence as set forth in any one of SEQ ID NOs 1-16 and SEQ ID NOs 40-42 are even more preferred. For example, antigenic peptides according to the present invention comprising or consisting of an amino acid sequence as set forth in any one of SEQ ID NOs 1, 2, 3, 7, 11, 16 are still more preferred. For example, antigenic peptides according to the present invention comprising or consisting of an amino acid sequence as set forth in any one of SEQ ID NOs 1-3 are still more preferred. Also combinations thereof are preferred, namely, nucleic acids encoding distinct antigenic peptides according to the present invention.

Nucleic acids preferably comprise single stranded, double stranded or partially double stranded nucleic acids, preferably selected from DNA, cDNA, PNA, RNA or combinations thereof. Non-limiting examples of nucleic acids include gDNA, cDNA, RNA, antisense DNA, antisense RNA, complementary RNA/DNA sequences with or without expression elements, a mini-gene, gene fragments, regulatory elements, promoters, and combinations thereof. Further examples of nucleic acids include, a recombinant polynucleotide, a vector, an oligonucleotide, an RNA molecule such as an rRNA, an mRNA, or a tRNA, or a DNA molecule as described above. It is thus preferred that the nucleic acid (molecule) is a DNA molecule or an RNA molecule; preferably selected from gDNA; cDNA; rRNA; mRNA; antisense DNA; antisense RNA; complementary RNA and/or DNA sequences; RNA and/or DNA sequences with or without expression elements, regulatory elements, and/or promoters; a vector; and combinations thereof.

It is of great interest in the fields of therapeutics, diagnostics, reagents and for biological assays to be able to deliver a nucleic acid, e.g., a ribonucleic acid (RNA) inside a cell, whether in vitro, in vivo, in situ or ex vivo, such as to cause intracellular translation of the nucleic acid and production of an encoded peptide of interest. Of particular importance is the delivery and function of a non-integrative polynucleotide. Accordingly, nucleic acids, which do not integrate into the chromosomes of the host, are preferred, such as mRNA. In general, nucleic acids, such as mRNA, may be optimized for expression of the antigenic peptide of the invention, e.g. by methods known in the art, such as codon optimization. In addition, the nucleic acid may be modified, for example, in order to enhance its stability, prolong its lifetime and/or to increase the expression of the antigenic peptide of the invention.

Accordingly, optimized or modified mRNA (mmRNA), which encodes an antigenic peptide according to the present invention, is preferred. The mmRNA are distinguished from wild type mRNA in their functional and/or structural design features for optimal delivery of the mRNA and/or for optimal expression of the antigenic peptide of the invention (for example as described in WO 2013/151672 A2, WO 2013/101690 A1, WO2013/052523 A, which are incorporated herein by reference). In general, nucleic acids may be delivered “naked” or associated with a carrier, e.g., a cationic carrier. Cationic carriers (positively charged) typically associate easily with nucleic acids, which are negatively charged. The carrier may be any of any kind including, for example, polymers, proteins, lipids and nanoparticles. Cationic lipids and nanoparticles (in particular lipid nanoparticles, LNPs) are preferred for nucleic acid delivery. Accordingly, the present invention also provides a nucleic acid as described herein associated with a carrier (e.g., a lipid, in particular a cationic lipid or an LNP).

In some embodiments, the nucleic acid molecule may be a vector. The term “vector”, as used in the context of the present invention, refers to a nucleic acid molecule, preferably to an artificial nucleic acid molecule, i.e. a nucleic acid molecule which does not occur in nature. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a desired antigenic peptide according to the present invention. An expression vector may be used for production of expression products such as RNA, e.g. mRNA, or peptides, polypeptides or proteins. For example, an expression vector may comprise sequences needed for transcription of a sequence stretch of the vector, such as a promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. Preferably, a vector is a DNA molecule. For example, a vector in the sense of the present application comprises a cloning site, a selection marker, such as an antibiotic resistance factor, and a sequence suitable for multiplication of the vector, such as an origin of replication. Preferably, a vector in the context of the present application is a plasmid vector. Preferably, a vector in the context of the present application is an expression vector. The expression vector is typically capable of expressing the encoded sequence, in particular the antigenic peptide according to the present invention, the polypeptide of formula (I) as defined above, or the immunogenic compound according to the present invention. A preferred vector is a vector for expression in bacterial cells. More preferably, the vector is useful for expression in so-called “live bacterial vaccine vectors”, wherein live bacterial cells (such as bacteria or bacterial spores, e.g., endospores, exospores or microbial cysts) can serve as vaccines. Preferred examples thereof are described in da Silva et al., Live bacterial vaccine vectors: an overview; Braz J Microbiol. 2015 Mar. 4; 45(4):1117-29.

Nucleic acids encoding antigenic peptides according to the invention may be in the form of naked nucleic acids, or nucleic acids cloned into plasmids or viral vectors (Tregoning and Kinnear, Using Plasmids as DNA Vaccines for Infectious Diseases. Microbiol Spectr. 2014 December; 2(6). doi: 10.1128/microbiolspec.PLAS-0028-2014), the latter being particularly preferred. Examples of suitable viral vectors according to the invention include, without limitation, retrovirus, adenovirus, adeno-associated virus (AAV), herpes virus and poxvirus vectors. It is within the skill of the person in the art to clone a nucleic acid into a plasmid or viral vector, using standard recombinant techniques in the art.

In a further aspect, the present invention also provides a host cell comprising the nucleic acid according to the present invention, in particular the or expression vector as described above.

Also combinations thereof are preferred, namely, host cells comprising distinct nucleic acids according to the present invention, for example encoding distinct antigenic peptides according to the present invention.

Preferably, the nucleic acid comprised in the host cell is preferably a vector. Preferably, the host cell is a bacterial cell. Such a host cell may be preferably used for production of the antigenic peptide according to the present invention or the immunogenic compound according to the present invention. Moreover, such a host cell may also be an active component in a vaccine. Preferably, the host cell is a bacterial cell, more preferably a gut bacterial cell. The term “gut bacterial cell” refers to bacteria residing in the (human) gut. Such a bacterial host cell may serve as “live bacterial vaccine vector”, wherein live bacterial cells (such as bacteria or bacterial spores, e.g., endospores, exospores or microbial cysts) can serve as vaccines. Preferred examples thereof are described in da Silva et al., Live bacterial vaccine vectors: an overview; Braz J Microbiol. 2015 Mar. 4; 45(4):1117-29. Bacterial cells (such as bacteria or bacterial spores, e.g., endospores, exospores or microbial cysts), in particular (entire) gut bacterial species, can be advantageous, as they have the potential to trigger a greater immune response than the (poly)peptides or nucleic acids they contain. Alternatively, bacterial cells, in particular gut bacteria, according to the invention may be in the form of probiotics, i.e. of live gut bacterium, which can thus be used as food additive due to the health benefits it can provide. Those can be for example lyophilized in granules, pills or capsules, or directly mixed with dairy products for consumption.

In some embodiments, the host cell may be an antigen presenting cell, and a dendritic cell, in particular as described above. In some embodiments, the antigen-presenting cell comprises an expression vector according to the invention as described above, in particular an expression vector capable of expressing or expressing said peptide containing SEQ ID NO: 1 to SEQ ID NO: 16 and SEQ ID NO: 40 to 42 or a variant amino acid sequence.

The present invention further relates to a method for producing a peptide according to the present invention, said method comprising culturing the host cell according to the present invention, and isolating the peptide from said host cell or its culture medium.

### Nanoparticles Comprising the Antigenic Peptide or the Immunogenic Compound

In a further aspect, the present invention also provides a nanoparticle comprising, in particular a nanoparticle loaded with,
at least one of the antigenic peptides according to the present invention, or
at least one of the immunogenic compounds according to the present invention;
   and, optionally, with an adjuvant.

In particular, preferred embodiments of the antigenic peptide as described above also apply for such a nanoparticle according to the present invention.

Nanoparticles, in particular for use as vaccines, are known in the art and described, for example, in Shao et al., Nanoparticle-based immunotherapy for cancer, ACS Nano 2015, 9(1):16-30; Zhao et al., Nanoparticle vaccines, Vaccine 2014, 32(3):327-37; and Gregory et al., Vaccine delivery using nanoparticles, Front Cell Infect Microbiol. 2013, 3:13, doi: 10.3389/fcimb.2013.00013. eCollection 2013, Review. In particular, the nanoparticle is used for delivery of the antigenic peptide (or the immunogenic compound/polypeptide/protein/nucleic acid comprising the antigenic peptide) and may optionally also act as an adjuvant. The antigenic peptide (the immunogenic compound/polypeptide/protein/nucleic acid comprising the antigenic peptide) is typically either encapsulated within the nanoparticle or linked/bound to (decorated onto) the surface of the nanoparticle (“coating”). Compared to conventional approaches, nanoparticles can protect the payload (antigen/adjuvant) from the surrounding biological milieu, increase the half-life, minimize the systemic toxicity, promote the delivery to APCs, or even directly trigger the activation of TAA-specific T-cells. Preferably, the nanoparticle has a size (diameter) of no more than 300 nm, more preferably of no more than 200 nm and most preferably of no more than 100 nm. Such nanoparticles are adequately sheltered from phagocyte uptake, with high structural integrity in the circulation and long circulation times, capable of accumulating at sites of tumor growth, and able to penetrate deep into the tumor mass.

Examples of nanoparticles include polymeric nanoparticles such as poly(ethylene glycol) (PEG) and poly (D,L-lactic-coglycolic acid) (PLGA); inorganic nanoparticles such as gold nanoparticles, iron oxide beads, iron-oxide zinc-oxide nanoparticles, carbon nanotubes and mesoporous silica nanoparticles; liposomes, such as cationic liposomes; immunostimulating complexes (ISCOM); virus-like particles (VLP); and self-assembled proteins.

Polymeric nanoparticles are nanoparticles based on/comprising polymers, such as poly(D,L-lactide-co-glycolide) (PLG), poly(D,L-lactic-coglycolic acid)(PLGA), poly(γ-glutamic acid) (γ-PGA), poly(ethylene glycol) (PEG), and polystyrene. Polymeric nanoparticles may entrap an antigen (e.g., the antigenic peptide or a (poly)peptide comprising the same) or bind to/conjugate to an antigen (e.g., the antigenic peptide or a (poly)peptide comprising the same).

Polymeric nanoparticles may be used for delivery, e.g. to certain cells, or sustain antigen release by virtue of their slow biodegradation rate. For example, g-PGA nanoparticles may be used to encapsulate hydrophobic antigens. Polystyrene nanoparticles can conjugate to a variety of antigens as they can be surface-modified with various functional groups. Polymers, such as Poly(L-lactic acid) (PLA), PLGA, PEG, and natural polymers such as polysaccharides may also be used to synthesize hydrogel nanoparticles, which are a type of nano-sized hydrophilic three-dimensional polymer network. Nanogels have favorable properties including flexible mesh size, large surface area for multivalent conjugation, high water content, and high loading capacity for antigens. Accordingly, a preferred nanoparticle is a nanogel, such as a chitosan nanogel. Preferred polymeric nanoparticles are nanoparticles based on/comprising PEG and PLGA.

Inorganic nanoparticles are nanoparticles based on/comprising inorganic substances, and examples of such nanoparticles include gold nanoparticles, iron oxide beads, iron-oxide zinc-oxide nanoparticles, carbon nanoparticles (e.g., carbon nanotubes) and mesoporous silica nanoparticles. Inorganic nanoparticles provide a rigid structure and controllable synthesis. For example, gold nanoparticles can be easily produced in different shapes, such as spheres, rods, cubes. Inorganic nanoparticles may be surface-modified, e.g. with carbohydrates. Carbon nanoparticles provide good biocompatibility and may be produced, for example, as nanotubes or (mesoporous) spheres. For example, multiple copies of the antigenic peptide according to the present invention (or a (poly)peptide comprising the same) may be conjugated onto carbon nanoparticles, e.g. carbon nanotubes. Mesoporous carbon nanoparticles are preferred for oral administration. Silica-based nanoparticles (SiNPs) are also preferred. SiNPs are biocompatible and show excellent properties in selective tumor targeting and vaccine delivery. The abundant silanol groups on the surface of SiNPs may be used for further modification to introduce additional functionality, such as cell recognition, absorption of specific biomolecules, improvement of interaction with cells, and enhancement of cellular uptake. Mesoporous silica nanoparticles are particularly preferred.

Liposomes are typically formed by phospholipids, such as 1,2-dioleoyl-3-trimethylammonium propane (DOTAP). In general, cationic liposomes are preferred. Liposomes are self-assembling with a phospholipid bilayer shell and an aqueous core. Liposomes can be generated as unilameller vesicles (having a single phospholipid bilayer) or as multilameller vesicles (having several concentric phospholipid shells separated by layers of water). Accordingly, antigens can be encapsulated in the core or between different layers/shells. Preferred liposome systems are those approved for human use, such as Inflexal® V and Epaxal®.

Immunostimulating complexes (ISCOM) are cage like particles of about 40 nm (diameter), which are colloidal saponin containing micelles, for example made of the saponin adjuvant Quil-A, cholesterol, phospholipids, and the (poly)peptide antigen (such as the antigenic peptide or a polypeptide comprising the same). These spherical particles can trap the antigen by apolar interactions. Two types of ISCOMs have been described, both of which consist of cholesterol, phospholipid (typically either phosphatidylethanolamine or phosphatidylcholine) and saponin (such as Quil-A).

Virus-like particles (VLP) are self-assembling nanoparticles formed by self-assembly of biocompatible capsid proteins. Due to the naturally-optimized nanoparticle size and repetitive structural order VLPs can induce potent immune responses. VLPs can be derived from a variety of viruses with sizes ranging from 20 nm to 800 nm, typically in the range of 20-150 nm. VLPs can be engineered to express additional peptides or proteins either by fusing these peptides/proteins to the particle or by expressing multiple antigens. Moreover, antigens can be chemically coupled onto the viral surface to produce bioconjugate VLPs.

Examples of self-assembled proteins include ferritin and major vault protein (MVP). Ferritin is a protein that can self-assemble into nearly-spherical 10 nm structure. Ninety-six units of MVP can self-assemble into a barrel-shaped vault nanoparticle, with a size of approximately 40 nm wide and 70 nm long. Antigens that are genetically fused with a minimal interaction domain can be packaged inside vault nanoparticles by self-assembling process when mixed with MVPs. Accordingly, the antigen (such as the antigenic peptide according to the present invention of a polypeptide comprising the same) may be fused to a self-assembling protein or to a fragment/domain thereof, such as the minimal interaction domain of MVP. Accordingly, the present invention also provides a fusion protein comprising a self-assembling protein (or a fragment/domain thereof) and the antigenic peptide according to the present invention.

In general, preferred examples of nanoparticles (NPs) include iron oxide beads, polystyrene microspheres, poly(γ-glutamic acid) (γ-PGA) NPs, iron oxide-zinc oxide NPs, cationized gelatin NPs, pluronic-stabilized poly(propylene sulfide) (PPS) NPs, PLGA NPs, (cationic) liposomes, (pH-responsive) polymeric micelles, PLGA, cancer cell membrane coated PLGA, lipid-calcium-phosphate (LCP) NPs, liposome-protamine-hyaluronic acid (LPH) NPs, polystyrene latex beads, magnetic beads, iron-dextran particles and quantum dot nanocrystals.

Preferably, the nanoparticle further comprises an adjuvant, for example a toll-like receptor (TLR) agonist. Thereby, the antigenic peptide (the immunogenic compound/polypeptide/protein/nucleic acid comprising the antigenic peptide) can be delivered together with an adjuvant, for example to antigen-presenting cells (APCs), such as dendritic cells (DCs). The adjuvant may be encapsulated by the nanoparticle or bound to/conjugated to the surface of the nanoparticle, preferably similarly to the antigenic peptide.

Particularly preferred adjuvants are polyinosinic:polycytidylic acid (also referred to as “poly I:C”) and/or its derivative poly-ICLC. Poly I:C is a mismatched double-stranded RNA with one strand being a polymer of inosinic acid, the other a polymer of cytidylic acid. Poly I:C is an immunostimulant known to interact with toll-like receptor 3 (TLR3). Poly I:C is structurally similar to double-stranded RNA, which is the “natural” stimulant of TLR3. Accordingly, poly I:C may be considered a synthetic analog of double-stranded RNA. Poly-ICLC is a synthetic complex of carboxymethylcellulose, polyinosinic-polycytidylic acid, and poly-L-lysine double-stranded RNA. Similar to poly I:C, also poly-ICLC is a ligand for TLR3. Poly I:C and poly-ICLC typically stimulate the release of cytotoxic cytokines. A preferred example of poly-ICLC is Hiltonol®.

Antibodies binding to the antigenic peptides in a further aspect, the present invention provides antibodies against (i.e. binding to) the antigenic peptides according to the present invention or complexes of said peptides according to the present invention with MHC.

As used herein, the term “antibody” encompasses various forms of antibodies including, without being limited to, whole antibodies, antibody fragments (such as antigen binding fragments), human antibodies, chimeric antibodies, humanized antibodies, recombinant antibodies and genetically engineered antibodies (variant or mutant antibodies) as long as the characteristic properties according to the invention (i.e. binding to the antigenic peptide) are retained. In some embodiments, the antibody is a mammal antibody, for example a murine, rat, rabbit, goat, sheep or human antibody. In some embodiments, the antibody is a monoclonal antibody. Antibodies usually comprise (at least) three complementarity determining regions (CDRs) on a heavy chain and (at least) three CDRs on a light chain. In general, complementarity determining regions (CDRs) are the hypervariable regions present in heavy chain variable domains and light chain variable domains. Typically, the CDRs of a heavy chain and the connected light chain of an antibody together form the antigen receptor.

Usually, the three CDRs (CDR1, CDR2, and CDR3) are arranged non-consecutively in the variable domain. Since antigen receptors are typically composed of two variable domains (on two different polypeptide chains, i.e. heavy and light chain: heavy chain variable region (VH) and light chain variable region (VL)), there are typically six CDRs for each antigen receptor (heavy chain: CDRH1, CDRH2, and CDRH3; light chain: CDRL1, CDRL2, and CDRL3). For example, a classical IgG antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a variable region (or each variable region, respectively) may be composed of four framework regions, separated by three CDR's. in addition thereto, the antibody may contain one or more constant regions. In some embodiments, the antibody comprises an Fc region.

Antibodies may be obtained, for example, by immunizing a mammal (e.g. mouse, rat, rabbit, goat, sheep or human) with the antigenic peptide of the invention (or the immunogenic compound or nanoparticle of the invention). Human antibodies may be isolated from a (isolated) sample (e.g., a blood sample) of a human.

Thus, the present invention also relates also to a method of immunizing a non-human animal with the antigenic peptide (or the immunogenic compound or nanoparticle) of the invention, the method comprising the following step:
contacting (immunizing) a non-human animal with the antigenic peptide (or the immunogenic compound or nanoparticle) of the present invention, preferably with the antigenic peptide according to any one of SEQ ID NOs 1 to 16 and SEQ ID NOs 40 to 42 (or the immunogenic compound or nanoparticle comprising such an antigenic peptide).

As used herein “immunizing” is understood to be of non-therapeutic nature, since it relates to the production of antibodies in said non-human animal.

The non-human animal is typically suitable for antibody production. Preferably, the non-human animal is a non-human mammal, more preferably an animal selected from goat and rodents such as mouse, rat, and rabbit.

The present invention relates also to a method of producing an (polyclonal) antibody recognizing the antigenic peptide of the invention, the method comprising the step of:
Isolating from a non-human animal, which has been contacted (immunized) previously with the antigenic peptide (or the immunogenic compound or nanoparticle) of the present invention,
   preferably with the antigenic peptide according to any one of SEQ ID NOs 1 to 16 and SEQ ID NOs 40 to 42 (or the immunogenic compound or nanoparticle comprising such an antigenic peptide),
an (polyclonal) antibody recognizing said antigenic peptide.

The present invention relates also to a method of isolating a cell producing an antibody recognizing the antigenic peptide according to the present invention, the method comprising the step of:
Isolating from a non-human animal, which has been contacted (immunized) previously with a JNK inhibitor of the present invention,
   preferably with the antigenic peptide according to any one of SEQ ID NOs 1 to 16 and SEQ ID NOs 40 to 42 (or the immunogenic compound or nanoparticle comprising such an antigenic peptide),
a cell producing said antibody recognizing said antigenic peptide, and optionally immortalizing said cell.

The present invention relates also to a method of producing a (monoclonal) antibody recognizing the antigenic peptide according to the present invention, the method comprising the step of:
Isolating an antibody recognizing the antigenic peptide of the present invention,
   more preferably recognizing the antigenic peptide according to any one of SEQ ID NOs 1 to 16 and SEQ ID NOs 40 to 42,
from the cell culture supernatant of a cell producing said antibody, the cell being optionally immortalized.

A person skilled in the art will understand, that the method of immunizing a non-human animal and the method of producing an (polyclonal) antibody as disclosed herein may be carried out consecutively. Similarly, the method of immunizing a non-human animal, the method of isolating a cell producing an antibody and the method of producing an (monoclonal) antibody may be combined.

In a further aspect the present invention relates to an antibody producible (and/or produced) with the methods according to the present invention for producing a polyclonal or monoclonal antibody, wherein the antibody recognizes at least one antigenic peptide of the invention, preferably the antigenic peptide according to any one of SEQ ID NOs: 1-16 and SEQ ID NO: 40-42. In some embodiments, said antibody does not recognize (bind to) the corresponding human peptide to a lesser extent.

The present invention also relates to a cell isolated according to the above specified method of isolating a cell producing an antibody recognizing the antigenic peptide according to the present invention, wherein the cell produces an antibody which preferably recognizes the antigenic peptide of the invention, preferably the antigenic peptide according to any one of SEQ ID NOs: 1-16 and SEQ ID NO: 40-42.

Methods for testing (monoclonal and/or polyclonal) antibodies for their binding affinities are well known in the art. One possibility among others is to characterize the binding affinity of an antibody by means of an ELISA by using the antigenic peptide of the invention as target peptide.

### T-Cell Receptors Binding to the Antigenic Peptides

The present invention further relates to T-cell receptors (TCRs), in particular soluble TCR (sTCRs) and cloned TCRs, which may be engineered into autologous or allogeneic T cells, and methods of making these, as well as NK cells or other cells bearing said TCR or cross-reacting with said TCRs.

As used herein, a “T-cell receptor” (TCR) is a protein complex found on the surface of T cells, (T lymphocytes), that is responsible for recognizing antigenic peptides bound to major histocompatibility complex (MHC) molecules.

To obtain T cells having a T cell receptor, which binds to the antigenic peptide of the invention, for example a (isolated) sample (e.g., a blood sample), e.g. of a mammal, such as a human, may be screened for binding to the antigenic peptide of the invention, such as a peptide according to any one of SEQ ID NOs 1-16 and SEQ ID NO: 40-42. Thereby, the antigenic peptide may be used as target peptide.

It is a further aspect of the invention to provide a method for producing a soluble T-cell receptor (sTCR) recognizing a specific peptide-MHC complex. Such soluble T-cell receptors can be generated from specific T-cell clones, and their affinity can be increased by mutagenesis targeting the complementarity-determining regions. For the purpose of T-cell receptor selection, phage display can be used (US 2010/0113300, Liddy N, et al. Monoclonal TCR-redirected tumor cell killing. Nat Med 2012 June; 18(6):980-987). For the purpose of stabilization of T-cell receptors during phage display and in case of practical use as drug, alpha and beta chain can be linked e.g. by non-native disulfide bonds, other covalent bonds (single-chain T-cell receptor), or by dimerization domains (see Boulter J M, et al. Stable, soluble T-cell receptor molecules for crystallization and therapeutics. Protein Eng 2003 September; 16(9):707-711; Card K F, et al. A soluble single-chain T-cell receptor IL-2 fusion protein retains MHC-restricted peptide specificity and IL-2 bioactivity. Cancer Immunol Immunother 2004 April; 53(4):345-357; and Willcox B E, et al. Production of soluble alphabeta T-cell receptor heterodimers suitable for biophysical analysis of ligand binding. Protein Sci 1999 November; 8 (11):2418-2423). The T-cell receptor can be linked to toxins, drugs, cytokines (see, for example, US 2013/0115191), domains recruiting effector cells such as an anti-CD3 domain, etc., in order to execute particular functions on target cells. Moreover, it could be expressed in T cells used for adoptive transfer. Further information can be found in WO 2004/033685A1 and WO 2004/074322A1. A combination of sTCRs is described in WO 2012/056407A1. Further methods for the production are disclosed in WO 2013/057586A1.

### Pharmaceutical Compositions

In a further aspect, the present invention also provides a pharmaceutical composition comprising at least one of the following:
the antigenic peptide according to the present invention as described herein,
the immunogenic compound according to the present invention as described herein,
the nanoparticle according to the present invention as described herein,
the cell according to the present invention as described herein,
the nucleic acid according to the present invention as described herein,
the host cell according to the present invention as described herein,
the T lymphocyte according to the present invention as described herein,
the antibody according to the present invention as described herein, and/or
the T cell receptor according to the present invention as described herein,
   and, optionally, one or more pharmaceutically acceptable excipients or carriers.

In particular, preferred embodiments of the antigenic peptide as described above also apply for such a pharmaceutical composition according to the present invention.

Also combinations thereof are preferred, namely, pharmaceutical compositions comprising distinct antigenic peptides according to the present invention. For example, the pharmaceutical composition may comprise
at least two distinct antigenic peptides according to the present invention as described herein,
at least two distinct immunogenic compounds according to the present invention as described herein,
at least two distinct nanoparticles according to the present invention as described herein,
at least two distinct cells according to the present invention as described herein,
at least two distinct nucleic acids according to the present invention as described herein,
at least two distinct host cells according to the present invention as described herein,
at least two distinct T lymphocytes according to the present invention as described herein,
at least two distinct antibodies according to the present invention as described herein, and/or
at least two distinct T cell receptors according to the present invention as described herein.

Accordingly, the pharmaceutical composition may comprise at least “two distinct components” (of a pharmaceutical composition according to the present invention), preferably three, four or five distinct components. In general, the expression “distinct components”, as used herein, refers to
(1) a first component, such as the antigenic peptide according to the present invention as described herein, the immunogenic compound according to the present invention as described herein, the nanoparticle according to the present invention as described herein, the cell according to the present invention as described herein, the nucleic acid according to the present invention as described herein, the host cell according to the present invention as described herein, the T lymphocyte according to the present invention as described herein, the antibody according to the present invention as described herein, and/or the T cell receptor according to the present invention as described herein; and
(2) at least one other component (which is distinct from the first component; while in the case of more than two distinct components each component is distinct from each other component), such as a distinct antigenic peptide according to the present invention as described herein, a distinct immunogenic compound according to the present invention as described herein, a distinct nanoparticle according to the present invention as described herein, a distinct cell according to the present invention as described herein, a distinct nucleic acid according to the present invention as described herein, a distinct host cell according to the present invention as described herein, a distinct T lymphocyte according to the present invention as described herein, a distinct antibody according to the present invention as described herein, and/or a distinct T cell receptor according to the present invention as described herein; or one or more (fragments of) human tumor antigens in any form (“naked”, as immunogenic compound as described herein, as nanoparticle as described herein, as (host) cell as described herein, or as nucleic acid as described herein).

The “distinct components” are preferably active components (as described above) in the context of a disease (cancer) to be prevented and/or treated. In other words, each of the distinct components may also be useful for prophylxais and/or treatment of said cancer, in particular if administered separately (not in combination as described herein)—although the combination (i.e. combined administration) typically potentiates their prophylactic and/or therapeutic effect (such as the immune response), preferably in a synergistic manner.

Preferably, the “distinct components” are of the same type (e.g., distinct antigenic peptides, distinct immunogenic compounds, distinct nanoparticles, distinct nucleic acids, distinct (host) cells, distinct T cell receptors, distinct antibodies, or distinct T lymphocytes) and differ from each other only in that they relate to distinct antigenic peptides of the present invention as described herein.

Preferably, the composition may comprise at least three, four or five distinct active components, which are preferably of the same type, but differ (only) in that each of them relates to a distinct antigenic peptide.

More preferably, the composition may comprise at least three, four or five distinct active components, which are preferably of the same type, but differ (only) in that each of them relates to a distinct antigenic peptide, wherein
the first component relating to the antigenic peptide comprising or consisting of a (microbiota) sequence variant of a fragment of the human tumor antigen CDC20;
the (distinct) second component relating to the antigenic peptide comprising or consisting of a (microbiota) sequence variant of a fragment of the human tumor antigen KIF2C; and
the (distinct) third component relating to the antigenic peptide comprising or consisting of a (microbiota) sequence variant of a fragment of the human tumor antigen UBE2C.

Preferably, such a composition may further comprise
a (distinct) fourth component relating to the antigenic peptide comprising or consisting of a (microbiota) sequence variant of a fragment of the human tumor antigen BIRC5; and/or
a (distinct) fifth component relating to the antigenic peptide comprising or consisting of a (microbiota) sequence variant of a fragment of the human tumor antigen FOXM1.

Table 2 below provides a list of additional peptides that may be useful in combination with the peptides of the invention (as described above). SEQ ID NOs 32 to 34 also refer to HLA-A*02 antigenic peptides.

TABLE 2
Additional HLA-A*02 antigenic peptides
useful in combination with the antigenic
peptides of the invention.

| | Sequence | SEQ ID NO. | | |
|---|---|---|---|---|
| | human | human | Sequence | SEQ ID NO. |
| Tumor | reference | reference | antigenic | antigenic |
| antigen | peptide | peptide | peptide | peptide |
| BIRC5 | LTLGEFLKL | 35 | FMLGEFLKL | 32 |
| FOXM1 | LMDLSTTPL | 36 | LMDLSTTEV | 33 |
| FOXM1 | RVSSYLVPI | 37 | RLSSYLVEI | 34 |

It is understood that these additional antigenic peptides for combination with the antigenic peptides of the invention may be provided in the same form, i.e. as antigenic peptides, immunogenic compounds, nanoparticles, cells loaded with the peptides, nucleic acids, host cells, T lymphocytes, antibodies or T cell receptors as described herein for the antigenic peptides of the present invention.

Accordingly, the pharmaceutical composition preferably comprises
a first component relating to the antigenic peptide comprising or consisting of a (microbiota) sequence variant of SEQ ID NO: 17;
a (distinct) second component relating to the antigenic peptide comprising or consisting of a (microbiota) sequence variant of SEQ ID NO: 18;
a (distinct) third component relating to the antigenic peptide comprising or consisting of a (microbiota) sequence variant of SEQ ID NO: 19;
optionally, a (distinct) fourth component relating to the antigenic peptide comprising or consisting of a (microbiota) sequence variant of SEQ ID NO: 35; and
optionally, a (distinct) fifth component relating to the antigenic peptide comprising or consisting of a (microbiota) sequence variant of SEQ ID NO: 36.

More preferably, the pharmaceutical composition preferably comprises
a first component relating to the antigenic peptide comprising or consisting of an amino acid sequence as set forth in SEQ ID NO: 1;
a (distinct) second component relating to the antigenic peptide comprising or consisting of an amino acid sequence as set forth in SEQ ID NO: 2;
a (distinct) third component relating to the antigenic peptide comprising or consisting of an amino acid sequence as set forth in SEQ ID NO: 3;
optionally, a (distinct) fourth component relating to the antigenic peptide comprising or consisting of an amino acid sequence as set forth in SEQ ID NO: 32, and
optionally, a (distinct) fifth component relating to the antigenic peptide comprising or consisting of an amino acid sequence as set forth in SEQ ID NO: 33.

Even more preferably, the pharmaceutical composition preferably comprises
the antigenic peptide comprising or consisting of an amino acid sequence as set forth in SEQ ID NO: 1;
the antigenic peptide comprising or consisting of an amino acid sequence as set forth in SEQ ID NO: 2;
the antigenic peptide comprising or consisting of an amino acid sequence as set forth in SEQ ID NO: 3;
optionally, the antigenic peptide comprising or consisting of an amino acid sequence as set forth in SEQ ID NO: 32, and
optionally, the antigenic peptide comprising or consisting of an amino acid sequence as set forth in SEQ ID NO: 33.

It is understood that the pharmaceutical composition may also contain—instead of the above-described preferred combinations of antigenic peptides—a respective combination of immunogenic compounds of the invention, a respective combination of nanoparticles of the invention, a respective combination of nucleic acids of the invention and so forth, as described above.

Preferably, the pharmaceutical composition further comprises one or more pharmaceutically acceptable excipients or carriers.

The pharmaceutical composition of the invention may be in any form suitable for the purposes of the invention. For example, said composition may be in a form suitable for parenteral, enteral or topical administration, such as a liquid suspension, a solid dosage form (granules, pills, capsules or tablets), or a paste or gel. It is within the skill of the person in the art to select the appropriate form of the composition for the intended purpose.

The composition according to the invention can further comprise other active agents, for example such, which can enhance the effects of the antigenic peptide or immunogenic compound. Alternatively, the composition may not comprise any other active agents (i.e., other than the antigenic peptide according to the present invention, the immunogenic compound according to the present invention, the nanoparticle according to the present invention, the cell according to the present invention, the nucleic acid according to the present invention, and/or the host cell according to the present invention).

The pharmaceutical composition as defined herein is preferably an immunogenic composition, i.e. a composition that is able to induce, increase, prolong or maintain an immune response. This may be achieved by an antigenic peptide according to the present invention or by an immunogenic compound according to the present invention comprised in said composition. Preferably, the pharmaceutical composition further comprises one or more immuno-adjuvant substances. A pharmaceutical composition, in particular an immunogenic composition, may also be termed “vaccine composition” in the present specification.

Preferably, the pharmaceutical composition further comprises at least one immunostimulatory agent, in particular so as to increase, potentiate, prolong or maintain the immune response mediated by the antigenic peptide. Preferred immunostimulatory agents according to the invention include, without limitation, immune adjuvants, antigen-presenting cells, and combinations thereof. Preferably, the immunostimulatory agent is an immune adjuvant or an antigen-presenting cell (APC).

Preferably, the immunostimulatory agent is an immune adjuvant. Some immune adjuvants are capable of favoring and prolonging the duration of interaction between an antigen and the immune system, while others are capable of recruiting and activating cells of the natural immunity so as to induce an adaptive response. The adjuvants belonging to the former category include, without limitation, mineral compounds such as alum, aluminum hydroxide, aluminum phosphate, calcium phosphate hydroxide; and oil-based emulsions such as paraffin oil, starch oil, Freund's complete/incomplete adjuvant (FCA/FIA), saponins (e.g. from the plants Quillaja, Soybean, Polygala senega). The adjuvants of belonging to the latter category include, without limitation, immunostimulatory complexes (ISCOMs) such as cytokines (e.g. GM-CSF; Interleukins such as IL-1, IL-2, IL6, IL8, or IL12; Tumor necrosis factors (TNFs) such as TNFα or TNFβ; Interferons IFNS such as IFNα, IFNβ, IFNγ or IFNδ, etc); ligands of toll-like receptors (TLRs) such as imiquimod, resiquimod or MPL; exosomes such as exosomes derived from dendritic cells (DCs) or from tumor cells; bacterial products such as heat-shock proteins (HSPs such as gp96, hsp90, hsp70, calreticulin, hsp110, hsp170), pathogen-associated molecular patterns (PAMPs), trehalose dimicolate (TDM), muramyldipeptide (MDP), polysaccharide (PLS) such as polysaccharide-K.

More preferably, the immune adjuvant is a protein/peptide having immuno-adjuvant properties, such as providing stimulation of CD4+Th1 cells, as described herein (“helper” peptides). This may be a non-tumor antigen that recalls immune memory or provides a non-specific help or could be a specific tumor-derived helper peptide, such as tetanus helper peptide, keyhole limpet hemocyanin peptide or PADRE peptide. Another example is a specific tumor derived helper peptide, which may be presented by MHC II, in particular by HLA-DR, HLA-DP or HLA-DQ, such as fragments of shared overexpressed tumor antigens, e.g. HER2, NY-ESO-1, hTERT or IL13RA2. In some embodiments, the immune adjuvant may be the HHD-DR3 peptide or h-pAg T13L (Bhasin M, Singh H, Raghava GP (2003) MHCBN: a comprehensive database of MHC binding and non-binding peptides. Bioinformatics 19: 665-666). Preferably, the helper peptides is the UCP2 peptide (SEQ ID NO: 39).

Preferably, the pharmaceutical composition comprises at least two distinct antigenic peptides according to the present invention and a helper peptide, preferably the UCP2 peptide (SEQ ID NO: 39).

Preferably, the pharmaceutical composition comprises a first antigenic peptide according to the present invention, which comprises or consists of a sequence variant of a fragment of the human tumor antigen CDC20, a second antigenic peptide according to the present invention, which comprises or consists of a sequence variant of a fragment of the human tumor antigen KIF2C, a third antigenic peptide according to the present invention, which comprises or consists of a sequence variant of a fragment of the human tumor antigen UBE2C, a fourth antigenic peptide according to the present invention, which comprises or consists of a sequence variant of a fragment of the human tumor antigen BIRC5, a fifth antigenic peptide according to the present invention, which comprises or consists of a sequence variant of a fragment of the human tumor antigen FOXM1 and a helper peptide.

More preferably, the pharmaceutical composition comprises a first antigenic peptide comprising or consisting of a sequence variant of the CDC20 fragment (human reference peptide) “SLPDRILDA” (SEQ ID NO: 17), such as an antigenic peptide comprising or consisting of an amino acid sequence as set forth in SEQ ID NO: 1; a second antigenic peptide comprising or consisting of a sequence variant of the KIF2C fragment (human reference peptide) “AINPELLQL” (SEQ ID NO: 18), such as an antigenic peptide comprising or consisting of an amino acid sequence as set forth in SEQ ID NO: 2; a third antigenic peptide comprising or consisting of a sequence variant of the UBE2C fragment (human reference peptide) “ALYDVRTIL” (SEQ ID NO: 19), such as an antigenic peptide comprising or consisting of an amino acid sequence as set forth in SEQ ID NO: 3; a fourth antigenic peptide comprising or consisting of a sequence variant of the BIRC5 fragment (human reference peptide) “LTLGEFLKL” (SEQ ID NO: 35); such as an antigenic peptide comprising or consisting of an amino acid sequence as set forth in SEQ ID NO: 32; a fifth antigenic peptide comprising or consisting of a sequence variant of the FOXM1 fragment (human reference peptide) “LMDLSTTPL” (SEQ ID NO: 36), such as an antigenic peptide comprising or consisting of an amino acid sequence as set forth in SEQ ID NO: 33; and a helper peptide, preferably the UCP2 peptide (SEQ ID NO: 39).

Even more preferably, the pharmaceutical composition comprises five distinct antigenic peptides, wherein the antigenic peptides comprise or consist of an amino acid sequence as set forth in SEQ ID NOs 1, 2, 3, 32 and 33. In addition, the pharmaceutical composition may further comprise the UCP2 peptide as set forth in SEQ ID NO: 39.

In some embodiments, the pharmaceutical composition does not comprise further antigenic peptides (in addition to the antigenic peptides as described above).

In some embodiments, the pharmaceutical composition comprises polyinosinic:polycytidylic acid (also referred to as “poly I:C”) and/or its derivative poly-ICLC (as immune adjuvants). Poly I:C is a mismatched double-stranded RNA with one strand being a polymer of inosinic acid, the other a polymer of cytidylic acid. Poly I:C is an immunostimulant known to interact with toll-like receptor 3 (TLR3). Poly I:C is structurally similar to double-stranded RNA, which is the “natural” stimulant of TLR3. Accordingly, poly I:C may be considered a synthetic analog of double-stranded RNA. Poly-ICLC is a synthetic complex of carboxymethylcellulose, polyinosinic-polycytidylic acid, and poly-L-lysine double-stranded RNA. Similar to poly I:C, also poly-ICLC is a ligand for TLR3. Poly I:C and poly-ICLC typically stimulate the release of cytotoxic cytokines. A preferred example of poly-ICLC is Hiltonol®.

More preferably, the pharmaceutical composition comprises Montanide, such as Montanide ISA 51 VG and/or Montanide ISA 720 VG. Those adjuvants are rendering stable water-in-oil emulsions when mixed with water based antigenic media. Montanide ISA 51 VG is based on a blend of mannide monooleate surfactant and mineral oil, whereas Montanide ISA 720 VG uses a non-mineral oil (Aucouturier J, Dupuis L, Deville S, Ascarateil S, Ganne V. Montanide ISA 720 and 51: a new generation of water in oil emulsions as adjuvants for human vaccines. Expert Rev Vaccines. 2002 June; 1(1):111-8; Ascarateil S, Puget A, Koziol M-E. Safety data of Montanide ISA 51 VG and Montanide ISA 720 VG, two adjuvants dedicated to human therapeutic vaccines. Journal for Immunotherapy of Cancer. 2015; 3(Suppl 2):P428. doi:10.1186/2051-1426-3-S2-P428).

In some embodiments, the pharmaceutical composition may further comprise at least one anti-cancer therapeutic agent. In another aspect, the invention relates to the combination of the pharmaceutical composition of the invention and at least one anti-cancer therapeutic agent. The pharmaceutical composition may comprise the anti-cancer therapeutic agent (as a combined preparation); or the pharmaceutical composition of the invention and the anti-cancer therapeutic agent may be provided in a separate manner, e.g. as a kit-of-parts. Accordingly, the pharmaceutical composition of the invention and the anti-cancer therapeutic agent may be provided for a simultaneous, separate, or sequential administration. In other terms, the invention proposes a combined use of the pharmaceutical composition the invention and least one anti-cancer therapeutic agent, for a simultaneous, separate, or sequential administration.

Said therapeutic agent is thus preferably capable of preventing and/or treating the same type of cancer than the one for which the antigenic peptide according to the invention is used. Preferably, the anti-cancer therapeutic agent is selected from antibodies, CAR-T cells, tumor cell lysates, chemotherapeutic agents, radiotherapeutic agents, immune checkpoint modulators and combinations thereof.

Antibodies are particularly advantageous in cancer therapy as they can either bind to specific antigens on cancer cell surfaces, thereby directing the therapy to the tumor (i.e. these are referred as tumor-targeting antibodies), or block immune checkpoints that are dysregulated in cancer (i.e. these are referred herein as immunomodulatory antibodies). The purpose of the later type of antibodies is to inhibit cancer immune resistance, which can notably be observed against T cells that are specific for tumor antigens. Indeed, as well-known in the art, under normal physiological conditions, immune checkpoints are crucial for the maintenance of self-tolerance (i.e. prevention of autoimmunity) and protect tissues from damage when the immune system is responding to pathogenic infection. However, in cancer, immune-checkpoints expression can be dysregulated as an important mechanism of immune resistance. Said resistance has notably been observed in melanoma, ovarian, lung, glioblastoma, breast, and pancreatic cancers with regard to the PD-L1 checkpoint (Konishi et al., B7-H1 expression on non-small cell lung cancer cells and its relationship with tumor-infiltrating lymphocytes and their PD-1 expression. Clin Cancer Res. 2004 Aug. 1; 10(15):5094-100; Ghebeh et al., The B7-H1 (PD-L1) T lymphocyte-inhibitory molecule is expressed in breast cancer patients with infiltrating ductal carcinoma: correlation with important high-risk prognostic factors. Neoplasia. 2006 March; 8(3):190-8; Hino et al., Tumor cell expression of programmed cell death-1 ligand 1 is a prognostic factor for malignant melanoma. Cancer. 2010 Apr. 1; 116(7):1757-66). Other examples of immune checkpoints include, without limitation, PD-L2, PD-1, CD80, CD86, CTLA-4, B7H3, B7H4, PVR, TIGIT, GAL9, LAG-3, GITR, CD137, TIM3, VISTA, VISTA-R (Pico de Coaia et al., Checkpoint blockade for cancer therapy: revitalizing a suppressed immune system. Trends Mol Med. 2015 August; 21(8):482-91; Pardoll DM. The blockade of immune checkpoints in cancer immunotherapy. Nat Rev Cancer. 2012 Mar. 22; 12(4):252-64).

Antibodies are usually employed for the above purposes either in the form of naked monoclonal antibodies (i.e. non-conjugated), or conjugated to another molecule which can be toxic to cells or radioactive.

Examples of well-known monoclonal tumor-targeting antibodies used in cancer immunotherapy include, without limitation, alemtuzumab (chronic lymphocytic leukemia), bevacizumab (colorectal cancer, glioblastoma multiforme, cervical cancer, lung cancer, renal cancer), brentuximab/vedotin (lymphomas), blinatumumab (acute lymphoblastic leukemia), catumaxomab (malignant ascites in EPCAM+ cancers), cetuximab (head and neck cancer, colorectal cancer), denosumab (breast, prostate and bone cancers), Gemtuzumab/ozogamicin (acute myeloid keulemia), ibritumomab/tiuxetan (non-Hodgkin lymphoma), panitumumab (colorectal cancer), pertuzumab (breast cancer), obinutuzumab (chronic lymphocytic leukemia), ofatumumab (chronic lymphocytic leukemia), ipilimumab (melanoma), ramucirumab (gastric and gastro-oeasophageal cancers), rituximab (chronic lymphocytic leukemia and non-Hodgkin lymphoma), siltuximab (multicentric's Catsleman's disease), tositumomab (non-Hodgkin lymphoma), and trastuzumab (breast, gastric and gastro-oeasophageal cancers); while examples of immunomodulatory antibodies include, without limitation, ipilimumab (melanoma) which blocks the CTLA4-dependent immune checkpoint, nivolumab (melanoma, lung cancer) and prembrolizubmab (melanoma) which both block the PDCD1-dependent immune checkpoint, as well as MPDL3280A, MEDI4736, MED10680, and MSB0010718C which all block the PD-L1-dependent immune checkpoint (Sharma and Allison, The future of immune checkpoint therapy. Science. 2015 Apr. 3; 348(6230):56-61).

Other antibodies for cancer immunotherapy have been described in Buqu6 et al., Trial Watch: Immunomodulatory monoclonal antibodies for oncological indications. Oncoimmunology. 2015 Mar. 2; 4(4):e1008814. eCollection 2015 April; Redman et al., Mechanisms of action of therapeutic antibodies for cancer. Mol Immunol. 2015 October; 67(2 Pt A):28-45; Simpson and Caballero, Monoclonal antibodies for the therapy of cancer MC Proc. 2014; 8(Suppl 4): 06 as well as on the antibody society website (list of therapeutic monoclonal antibodies approved or in review in the European Union or United States available on the weblink http://www.antibodysociety.org/news/approved_mabs.php).

Adoptive cellular immunotherapy with chimeric antigen receptor (CAR) T cells has changed the treatment landscape of B-cell non-Hodgkin's lymphoma (NHL), especially for aggressive B-cell lymphomas. For instance, CD19-targeted CAR T-cells, represent the new standard of care for patients with DLBCL that are refractory to at least two prior lines of therapy. Two CAR T-cell products axicabtagene ciloleucel (axi-cel) (KTE-019) (YESCARTAT™) and tisagenlecleucel (CTL019) (KYMRIAH™) have obtained US Food and Drug Administration approval for the treatment of refractory DLBCL after two lines of therapy. A third product, lisocabtagene maraleucel (liso-cel) (JCAR017), is currently being evaluated in clinical trials. Other CAR T-cells include CD20-CAR-T cells.

Tumor cell lysates may also be combined with the antigenic peptide(s) according to the invention. Tumor cells are indeed capable of priming the immune response, by presenting endogenous peptides-MHC complexes, as well as via dendritic cells (DCs) of the host which can process and present the antigen delivered by said lysates. The range of antigens against which an immune response can be induced is thereby increased. Tumor cell lysates can be easily obtained by treating tumor cells with a heat shock and/or a chemical treatment, and can be autologous (i.e. isolated from the patient), or allogeneic (i.e. isolated from another subject).

Standard chemotherapeutic drugs and radiotherapeutic agents need not be further described herein as they have been extensively described in the literature, notably by Baskar et al. (Baskar et al., Cancer and radiation therapy: current advances and future directions. Int J Med Sci. 2012; 9(3):193-9), Paci et al., (Paci et al., Review of therapeutic drug monitoring of anticancer drugs part 1—cytotoxics. Eur J Cancer. 2014 August; 50(12):2010-9) and Widmer et al. (Widmer et al., Review of therapeutic drug monitoring of anticancer drugs part two—targeted therapies. Eur J Cancer. 2014 August; 50(12):2020-36). A list of such drugs and agents is also available on the cancer.gov website (http//www.cancer.gov/about-cancer/treatment/drugs).

Preferably, the immune checkpoint modulator for combination with the antigenic peptide as defined herein is an activator or an inhibitor of one or more immune checkpoint point molecule(s) selected from CD27, CD28, CD40, CD122, CD137, OX40, GITR, ICOS, A2AR, B7-H3, B7-H4, BTLA, CD40, CTLA-4, IDO, KIR, LAG3, PD-1, TIM-3, VISTA, CEACAM1, GARP, PS, CSF1R, CD94/NKG2A, TDO, GITR, TNFR and/or FasR/DcR3; or an activator or an inhibitor of one or more ligands thereof.

More preferably, the immune checkpoint modulator is an activator of a (co-)stimulatory checkpoint molecule or an inhibitor of an inhibitory checkpoint molecule or a combination thereof. Accordingly, the immune checkpoint modulator is more preferably (i) an activator of CD27, CD28, CD40, CD122, CD137, OX40, GITR and/or ICOS or (ii) an inhibitor of A2AR, B7-H3, B7-H4, BTLA, CD40, CTLA-4, IDO, KIR, LAG3, PD-1, PDL-1, PD-L2, TIM-3, VISTA, CEACAM1, GARP, PS, CSF1R, CD94/NKG2A, TDO, TNFR and/or FasR/DcR3.

Even more preferably, the immune checkpoint modulator is an inhibitor of an inhibitory checkpoint molecule (but preferably no inhibitor of a stimulatory checkpoint molecule). Accordingly, the immune checkpoint modulator is even more preferably an inhibitor of A2AR, B7-H3, B7-H4, BTLA, CTLA-4, DO, KIR, LAG3, PD-1, PDL-1, PD-L2, TIM-3, VISTA, CEACAM1, GARP, PS, CSF1R, CD94/NKG2A, TDO, TNFR and/or DcR3 or of a ligand thereof.

Preferably, the checkpoint modulator for combination with the antigenic peptide as defined herein may be selected from known modulators of the CTLA-4 pathway or the PD-1 pathway. More preferably, the immune checkpoint modulator is an inhibitor of CTLA-4, PD-L1, PD-L2, or PD-1; even more preferably an inhibitor of the PD-1 pathway.

It is within the skill of ordinary person in the art to select the appropriate immune anti-cancer therapeutic agent for the purposes of the invention. For example, should one wish to prevent or treat melanoma, a lysate from melanoma cells and/or the antibody Ipilimumab can preferably be used, along with an appropriate antigenic peptide. Appropriate antigenic peptides may be selected by (i) selecting an appropriate tumor antigen for a certain type of cancer as known in the art and (ii) selecting an appropriate antigenic peptide according to the invention for the selected tumor antigen, as described above, e.g. in Table 1.

The anti-cancer therapeutic agent can also be administered in combination with the composition of the invention, either simultaneously, separately, or sequentially. Should the composition and the therapeutic agent be administered in a separate or sequential manner, those may be administered in distinct pharmaceutical forms.

Thus, in another aspect, the invention relates to a composition of the invention and at least one anti-cancer therapeutic agent as described above, as a combined preparation for a simultaneous, separate, or sequential administration. In other terms, the invention proposes a combined use of the composition the invention and least one anti-cancer therapeutic agent as described above, for a simultaneous, separate, or sequential administration.

### Kits-of-Parts

In a further aspect, the present invention also provides a kit-of-parts (also referred to herein as “kit”) comprising at least one of the following:
the antigenic peptide according to the present invention as described herein,
the immunogenic compound according to the present invention as described herein,
the nanoparticle according to the present invention as described herein,
the cell according to the present invention as described herein,
the nucleic acid according to the present invention as described herein,
the host cell according to the present invention as described herein,
the T lymphocyte according to the present invention as described herein,
the antibody according to the present invention as described herein,
the T cell receptor according to the present invention as described herein, and/or
the pharmaceutical composition according to the present invention as described herein.

In particular, preferred embodiments of the antigenic peptide as described above also apply for such a kit according to the present invention.

Also combinations thereof are preferred, namely, kits comprising distinct antigenic peptides according to the present invention. In particular, the kit-of-parts of the invention may comprise more than one of the above described components, e.g. 2, 3, 4, 5, 6, 7, 8, 9, or 10 distinct components. For example, the kit-of-parts according to the present invention may comprise at least two (e.g. 2, 3, 4, 5, 6, 7, 8, 9, or 10) different immunogenic compounds, at least two (e.g. 2, 3, 4, 5, 6, 7, 8, 9, or 10) different antigenic peptides, at least two (e.g. 2, 3, 4, 5, 6, 7, 8, 9, or 10) different nanoparticles, at least two (e.g. 2, 3, 4, 5, 6, 7, 8, 9, or 10) different cells, at least two (e.g. 2, 3, 4, 5, 6, 7, 8, 9, or 10) different nucleic acids, at least two (e.g. 2, 3, 4, 5, 6, 7, 8, 9, or 10) different host cells, at least two (e.g. 2, 3, 4, 5, 6, 7, 8, 9, or 10) different pharmaceutical compositions and the like. Preferably, such different components comprised in the kit-of-parts as described above differ in the antigenic peptides according to the present invention, for example one component relating to a first antigenic peptide, and one component relating to a second antigenic peptide (distinct from the first antigenic peptide). For example, the kit may comprise at least two distinct immunogenic compounds according to the present invention. For example, the kit may comprise at least two distinct antigenic peptides according to the present invention. For example, the kit may comprise at least two distinct nanoparticles according to the present invention. For example, the kit may comprise at least two distinct nucleic acids according to the present invention. For example, the kit may comprise at least two distinct cytotoxic T lymphocytes according to the present invention.

Preferred combinations of components, such as antigenic peptides, according to the present invention included in the kit correspond to the preferred combinations of components, such as antigenic peptides, according to the present invention included in the pharmaceutical composition as described above.

Accordingly, the present invention provides a kit comprising at least two, preferably three, more preferably four or five distinct antigenic peptides as described herein (or immunogenic compounds, nanoparticles, nucleic acids, cells, etc. as described above, which differ regarding the antigenic peptide), and, optionally, a helper peptide, such as the UCP2 peptide of SEQ ID NO: 39, and/or an adjuvant, such as MONTANIDE ISA 51.

Preferably, the kit comprises distinct antigenic peptides (in form of peptides or in any other form as described above), including (i) at least one antigenic peptide of the present invention, e.g. as shown in Table 1; and (ii) at least one additional antigenic peptide as described above, e.g. as shown in Table 2 above.

In some embodiments, the kit comprises
a first component relating to the antigenic peptide comprising or consisting of a (microbiota) sequence variant of a fragment of the human tumor antigen CDC20;
a (distinct) second component relating to the antigenic peptide comprising or consisting of a (microbiota) sequence variant of a fragment of the human tumor antigen KIF2C;
a (distinct) third component relating to the antigenic peptide comprising or consisting of a (microbiota) sequence variant of a fragment of the human tumor antigen UBE2C;
optionally, a (distinct) fourth component relating to the antigenic peptide comprising or consisting of a (microbiota) sequence variant of a fragment of the human tumor antigen BIRC5; and
optionally, a (distinct) fifth component relating to the antigenic peptide comprising or consisting of a (microbiota) sequence variant of a fragment of the human tumor antigen FOXM1.

Preferably, the kit comprises
a first component relating to the antigenic peptide comprising or consisting of a (microbiota) sequence variant of SEQ ID NO: 17;
a (distinct) second component relating to the antigenic peptide comprising or consisting of a (microbiota) sequence variant of SEQ ID NO: 18;
a (distinct) third component relating to the antigenic peptide comprising or consisting of a (microbiota) sequence variant of SEQ ID NO: 19;
optionally, a (distinct) fourth component relating to the antigenic peptide comprising or consisting of a (microbiota) sequence variant of SEQ ID NO: 35; and
optionally, a (distinct) fifth component relating to the antigenic peptide comprising or consisting of a (microbiota) sequence variant of SEQ ID NO: 36.

More preferably, the kit comprises
a first component relating to the antigenic peptide comprising or consisting of an amino acid sequence as set forth in SEQ ID NO: 1;
a (distinct) second component relating to the antigenic peptide comprising or consisting of an amino acid sequence as set forth in SEQ ID NO: 2;
a (distinct) third component relating to the antigenic peptide comprising or consisting of an amino acid sequence as set forth in SEQ ID NO: 3;
optionally, a (distinct) fourth component relating to the antigenic peptide comprising or consisting of an amino acid sequence as set forth in SEQ ID NO: 32, and
optionally, a (distinct) fifth component relating to the antigenic peptide comprising or consisting of an amino acid sequence as set forth in SEQ ID NO: 33.

It is understood that the kit may also contain—instead of the above-described preferred combinations of antigenic peptides—a respective combination of immunogenic compounds of the invention, a respective combination of nanoparticles of the invention, a respective combination of nucleic acids of the invention and so forth, as described above.

Preferably, the kit further comprises the UCP2 helper peptide of SEQ ID NO: 39.

In some embodiments, in addition to any of components as described above, the kit further comprises an anti-cancer therapeutic agent as described above.

Said therapeutic agent is thus preferably capable of preventing and/or treating the same type of cancer than the one for which the antigenic peptide according to the invention is used. Preferably, the anti-cancer therapeutic agent is selected from antibodies as described above, CAR-T cells as described above, tumor cell lysates as described above, chemotherapeutic agents as described above, radiotherapeutic agents as described above, immune checkpoint modulators as described above and combinations thereof.

The various components of the kit-of-parts may be packaged in one or more containers. The above components may be provided in a lyophilized or dry form or dissolved in a suitable buffer. The kit may also comprise additional reagents including, for instance, preservatives, growth media, and/or buffers for storage and/or reconstitution of the above-referenced components, washing solutions, and the like.

Distinct antigenic peptides (or immunogenic compounds, nanoparticles, nucleic acids, cells, etc. as described above, which differ regarding the antigenic peptide) may be contained in the same or in distinct containers. For example, the kit may comprise a (single) container containing a first antigenic peptide as described herein and a second antigenic peptide as described herein. Said (single) container may additionally also comprise a helper peptide, such as UCP2. Optionally, the first and second antigenic peptide (and optionally the helper peptide) contained in the (single) container may be formulated together, e.g. in water for injection and/or Dimethyl sulfoxide (DMSO). Additionally, the kit may comprise a further container (distinct from the container containing the antigenic peptides), which contains the adjuvant, e.g. MONTANIDE ISA 51.

Optionally, the kit can also comprise a vial of water for injection and/or a vial adapter. A sterile needle can also be comprised, e.g. for vaccinating the patient after obtaining the emulsion. The kit may also comprise one or more syringes.

In addition, the kit-of-parts according to the present invention may optionally contain instructions of use. Accordingly, it is preferred that the kit comprises a package insert or instruction leaflet with directions for prophylaxis or treatment of a cancer by using the immunogenic compound according to the present invention, the antigenic peptide according to the present invention, the nanoparticle according to the present invention, the cell according to the present invention, the nucleic acid according to the present invention, the host cell according to the present invention, the pharmaceutical composition according to the present invention, and so on, as described above.

Moreover, the present invention also provides a vaccination kit for treating, preventing and/or stabilizing a cancer, comprising the pharmaceutical composition as described herein or a vaccine as described herein and instructions for use of said pharmaceutical composition or of said vaccine in the prophylaxis and/or treatment of a cancer.

### Medical Treatment and Uses

As stated above, the antigenic peptides of the invention (in its many different forms as described above) can be particularly useful for prophylactic or therapeutic purposes (as medicament), notably for triggering a specific immune response towards a particular tumor antigen/protein, for example for prophylaxis or treatment of a cancer, e.g. in a patient in need thereof.

In view thereof, the present invention provides
the antigenic peptide according to the present invention as described herein,
the immunogenic compound according to the present invention as described herein,
the nanoparticle according to the present invention as described herein,
the cell according to the present invention as described herein,
the nucleic acid according to the present invention as described herein,
the host cell according to the present invention as described herein,
the T lymphocyte according to the present invention as described herein,
the antibody according to the present invention as described herein, and/or
the T cell receptor according to the present invention as described herein.
the pharmaceutical composition according to the present invention as described herein, or
the kit according to the present invention as described herein, for use in medicine, in particular in the prophylaxis and/or in the treatment of proliferative diseases, preferably in the prophylaxis and/or in the treatment of a cancer.

Moreover, the present invention also provides a method for preventing (reducing occurrence of) and/or treating a cancer or initiating, enhancing or prolonging an anti-tumor-response in a subject (in need thereof), comprising administering to the subject (an effective amount of)
the antigenic peptide according to the present invention as described herein,
the immunogenic compound according to the present invention as described herein,
the nanoparticle according to the present invention as described herein,
the cell according to the present invention as described herein,
the nucleic acid according to the present invention as described herein,
the host cell according to the present invention as described herein,
the T lymphocyte according to the present invention as described herein,
the antibody according to the present invention as described herein, and/or
the T cell receptor according to the present invention as described herein.
the pharmaceutical composition according to the present invention as described herein, or
the kit according to the present invention as described herein.

In particular, preferred embodiments of the antigenic peptide as described above also apply for the use according to the present invention in the prophylaxis and/or in the treatment of a cancer. Non-limiting examples of cancers include colorectal cancer, lung cancer, prostate cancer and/or breast cancer.

Moreover, the present invention provides a method for eliciting or improving, in a subject, an immune response against one or multiple epitopes that is dependent on CD8⁺ cytotoxic T cells, wherein said method comprises administering to said subject any one of:
the antigenic peptide according to the present invention as described herein,
the immunogenic compound according to the present invention as described herein,
the nanoparticle according to the present invention as described herein,
the cell according to the present invention as described herein,
the nucleic acid according to the present invention as described herein,
the host cell according to the present invention as described herein,
the T lymphocyte according to the present invention as described herein,
the antibody according to the present invention as described herein, and/or
the T cell receptor according to the present invention as described herein.
the pharmaceutical composition according to the present invention as described herein, or
the kit according to the present invention as described herein.

An immune response that is dependent on CD8⁺ response can be determined by evaluating an inflammatory response, a pro-inflammatory cytokine response, including an increase in the expression of one or more of IFN-γ, TNF-α and IL-2 mRNA or protein relative to the level before administration of the compounds of the invention. It can also be measured by an increase in the frequency or absolute number of antigen-specific T cells after administration of the compounds of the invention, measured by HLA-peptide multimer staining, ELISPOT assays, and delayed type hypersensitivity tests. It can also be indirectly measured by an increase in antigen-specific serum antibodies that are dependent on antigen-specific T helper cells.

The present invention also provides a method for eliciting or improving, in a subject, an immune response against one or multiple antigens or antigenic epitopes that is restricted by multiple MHC class I molecules, wherein said method comprises administering to said subject any one of:
the antigenic peptide according to the present invention as described herein,
the immunogenic compound according to the present invention as described herein,
the nanoparticle according to the present invention as described herein,
the cell according to the present invention as described herein,
the nucleic acid according to the present invention as described herein,
the host cell according to the present invention as described herein,
the T lymphocyte according to the present invention as described herein,
the antibody according to the present invention as described herein, and/or
the T cell receptor according to the present invention as described herein.
the pharmaceutical composition according to the present invention as described herein, or
the kit according to the present invention as described herein.

A method for eliciting or improving, in a subject, an immune response against multiple epitopes as described herein, that is restricted by multiple MHC class I molecules can be determined by evaluating a cytokine response, including an increase in the expression of one or more of IFN-γ, TNF-α and IL-2 mRNA or protein relative to the level before administration of the compounds of the invention, after in vitro stimulation of T cells with individual peptides binding to discrete MHC class I molecules on antigen presenting cells. Restriction to MHC class I molecules can also be validated by using antigen presenting cells expressing MHC class I molecules, or by using MHC class I blocking antibodies. It can also be measured by an increase in the frequency or absolute number of antigen-specific T cells after administration of the compounds of the invention, measured by HLA-peptide multimer staining, using multimers assembled with MHC class I molecules.

The invention relates more particularly to a composition as defined above, for use as a vaccine for immunotherapy. Moreover,
the antigenic peptide according to the present invention as described herein,
the immunogenic compound according to the present invention as described herein,
the nanoparticle according to the present invention as described herein,
the cell according to the present invention as described herein,
the nucleic acid according to the present invention as described herein,
the host cell according to the present invention as described herein,
the T lymphocyte according to the present invention as described herein,
the antibody according to the present invention as described herein, and/or
the T cell receptor according to the present invention as described herein.
the pharmaceutical composition according to the present invention as described herein, or
the kit according to the present invention as described herein
   may be used as vaccine, in particular for (cancer) immunotherapy.

As used in the context of the present invention, the term “vaccine” refers to a (biological) preparation that provides innate and/or adaptive immunity, typically to a particular disease, preferably a cancer. Thus, a vaccine supports in particular an innate and/or an adaptive immune response of the immune system of a subject to be treated. For example, the antigenic peptide according to the present invention typically leads to or supports an adaptive immune response in the patient to be treated.

In the context of the present invention, the vaccine (composition) can induce a specific immune response against a tumor antigen, and is thus preferably used for prophylaxis or treatment of a cancer.

Accordingly, in a preferred embodiment, the invention relates to a composition as defined above, for use in the prevention and/or treatment of cancer in a subject in need thereof. More preferably, the invention relates to the use of the composition of the invention for manufacturing a medicament to prevent or treat cancer in a subject in need thereof. In other words, the invention relates to a method for preventing or treating cancer in a subject in need thereof, comprising administering an effective amount of the composition of the invention, to said subject.

Preferably the cancer to be prevented and/or treated by
the antigenic peptide according to the present invention as described herein,
the immunogenic compound according to the present invention as described herein,
the nanoparticle according to the present invention as described herein,
the cell according to the present invention as described herein,
the nucleic acid according to the present invention as described herein,
the host cell according to the present invention as described herein,
the T lymphocyte according to the present invention as described herein,
the antibody according to the present invention as described herein, and/or
the T cell receptor according to the present invention as described herein.
the pharmaceutical composition according to the present invention as described herein, or
the kit according to the present invention as described herein.
   relates to the (reference) tumor antigen of the antigenic peptide as described herein. Namely, appropriate antigenic peptides may be selected by (i) selecting an appropriate tumor antigen for a certain type of cancer as known in the art and (ii) selecting an appropriate antigenic peptide according to the invention for the selected tumor antigen, as described above, e.g. in Table 1 (and, optionally in addition thereto, Table 2). One skilled in the art will readily understand that an antigenic peptide of the invention can be selected based upon the nature of the cancer to be prevented or treated, and/or on the human gene/human tumor antigen involved in said cancer.

Particularly preferred for prophylaxis or treatment of a cancer are the peptides—alone or in combination—according to the present invention selected from the group consisting of SEQ ID NO: 1 to SEQ ID NO: 16 and SEQ ID NO: 40 to SEQ ID NO: 42. More preferred are the peptides—alone or in combination—selected from the group consisting of SEQ ID NO: 1 to SEQ ID NO: 16 and SEQ ID NO: 40 to SEQ ID NO: 42 (see Table 1), optionally in combination with at least one peptide selected from the group consisting of SEQ ID NO: 32 to SEQ ID NO: 34 (see Table 2), and their uses in the immunotherapy of colorectal cancer, lung cancer, prostate cancer and/or breast cancer.

Thus, another aspect of the present invention relates to the use of at least one peptide according to any one of SEQ ID No. 1 to 16 and SEQ ID NO: 40 to SEQ ID NO: 42 for the—preferably combined—treatment of a proliferative disease selected from the group of colorectal cancer, lung cancer, prostate cancer and breast cancer.

Thus, another aspect of the present invention relates to the use of the peptides according to the present invention for the—preferably combined—treatment of a proliferative disease, in particular a cancer, for example selected from the group of colorectal cancer, lung cancer, prostate cancer and breast cancer.

As described above, in the context of the pharmaceutical composition and the kit of the invention, combinations of the above items are preferred, namely, distinct antigenic peptides according to the present invention are preferably for use in the prophylaxis and/or in the treatment of a cancer. Preferably, more than one of the above described components may be used in the prophylaxis and/or in the treatment of a cancer. For example, at least two different antigenic peptides, at least two different immunogenic compounds, at least two different nanoparticles, at least two different cells, at least two different nucleic acids, at least two different host cells, at least two different pharmaceutical compositions etc. may be used in the prophylaxis and/or in the treatment of a cancer. Preferably, such different components used in the prophylaxis and/or in the treatment of a cancer differ in the antigenic peptides according to the present invention, for example one component relating to a first antigenic peptide, and one component relating to a second antigenic peptide (distinct from the first antigenic peptide), as described above, for example in the context of the pharmaceutical composition or the kit.

Accordingly, the present invention also provides a combination of at least two distinct antigenic peptides according to the present invention for use in the prophylaxis or treatment of cancer.

For prophylaxis or treatment of a cancer, such as colorectal cancer, lung cancer, prostate cancer and/or breast cancer, the combinations of antigenic peptides as described above (in the context of the pharmaceutical composition or the kit) are preferred.

In some embodiments,
a first component relating to the antigenic peptide comprising or consisting of a (microbiota) sequence variant of a fragment of the human tumor antigen CDC20;
a (distinct) second component relating to the antigenic peptide comprising or consisting of a (microbiota) sequence variant of a fragment of the human tumor antigen KIF2C;
a (distinct) third component relating to the antigenic peptide comprising or consisting of a (microbiota) sequence variant of a fragment of the human tumor antigen UBE2C;
optionally, a (distinct) fourth component relating to the antigenic peptide comprising or consisting of a (microbiota) sequence variant of a fragment of the human tumor antigen BIRC5; and
optionally, a (distinct) fifth component relating to the antigenic peptide comprising or consisting of a (microbiota) sequence variant of a fragment of the human tumor antigen FOXM1 may be administered (in combination, i.e. simultaneously or consecutively) for prophylaxis and/or treatment of a cancer.

Preferably,
a first component relating to the antigenic peptide comprising or consisting of a (microbiota) sequence variant of SEQ ID NO: 17;
a (distinct) second component relating to the antigenic peptide comprising or consisting of a (microbiota) sequence variant of SEQ ID NO: 18;
a (distinct) third component relating to the antigenic peptide comprising or consisting of a (microbiota) sequence variant of SEQ ID NO: 19;
optionally, a (distinct) fourth component relating to the antigenic peptide comprising or consisting of a (microbiota) sequence variant of SEQ ID NO: 35; and
optionally, a (distinct) fifth component relating to the antigenic peptide comprising or consisting of a (microbiota) sequence variant of SEQ ID NO: 36
   are administered (in combination, i.e. simultaneously or consecutively) for prophylaxis and/or treatment of a cancer.

More preferably,
a first component relating to the antigenic peptide comprising or consisting of an amino acid sequence as set forth in SEQ ID NO: 1;
a (distinct) second component relating to the antigenic peptide comprising or consisting of an amino acid sequence as set forth in SEQ ID NO: 2;
a (distinct) third component relating to the antigenic peptide comprising or consisting of an amino acid sequence as set forth in SEQ ID NO: 3;
optionally, a (distinct) fourth component relating to the antigenic peptide comprising or consisting of an amino acid sequence as set forth in SEQ ID NO: 32, and
optionally, a (distinct) fifth component relating to the antigenic peptide comprising or consisting of an amino acid sequence as set forth in SEQ ID NO: 33
   are administered (in combination, i.e. simultaneously or consecutively) for prophylaxis and/or treatment of a cancer.

Methods of administration are well-known to the skilled person in the art. With regard to the composition of the invention, it can be directly administered into the subject, into the affected organ (i.e. local administration) or systemically (i.e. enteral or parenteral administration), or even applied ex vivo to cells derived from the subject or a human cell line which are subsequently administered to the subject, or even used in vitro to select a subpopulation of immune cells derived from the subject, which are then re-administered to the said subject. Enteral administrations include oral and rectal administrations, as well as administrations via gastric feeding tubes, duodenal feeding tubes or gastrostomy, while parenteral administrations include, among others, subcutaneous, intravenous, intramuscular, intra-arterial, intradermal, intraosseous, intracerebral, and intrathecal injections. The administration method will often depend upon the antigenic peptide(s) and/or immunogenic compound(s) present in the composition, and the type of cancer to be treated and other active agents that may be contained in said composition. For example, the administration is preferably an intramuscular or an intradermal injection if the immunogenic compound is a nucleic acid as defined above, the oral/nasal administration being particularly preferred if said nucleic acid is cloned into a viral vector. Alternatively, the administration is preferably an intramuscular, an intradermal or an oral administration if the antigenic peptide and/or immunogenic compound is a (poly)peptide as defined above or if it is loaded in/on a nanoparticle as described herein. Yet, still alternatively, the administration is preferably an oral administration if the antigenic peptide and/or immunogenic compound is delivered in the form of a gut bacterium as defined above, notably if the gut bacterium is in the form of probiotics.

The antigenic peptides, the immunogenic compounds and the nucleic acids according to the invention can further be encapsulated so as to facilitate their administration to the subject in need thereof. For example, those may be encapsulated into peptide nanocarriers (preferable if the immunogenic compound is a nucleic acid or a (poly)peptide), into virosomes (preferable if the immunogenic compound is a nucleic acid or a (poly)peptide), or into lipid-based carrier systems such as liposome-polycation-DNA complex (preferable if the immunogen is a nucleic acid or a (poly)peptide) (Trovato M, De Berardinis P. Novel antigen delivery systems. World J Virol. 2015 Aug. 12; 4(3):156-68; Saade F, Petrovsky N. Technologies for enhanced efficacy of DNA vaccines. Expert Rev Vaccines. 2012 February; 11(2):189-209; Li et al., Peptide Vaccine: Progress and Challenges. Vaccines (Basel). 2014 Jul. 2; 2(3):515-36).

The composition may also be administered more than once so as to achieve the desired effect. In a preferred embodiment, said composition is administered repeatedly, at least twice, and preferably more than twice. This can be done over an extended period of time, such as weekly, every other week, monthly, yearly, or even several years after the first administration to ensure that the subject is properly immunized.

The present invention further relates to a method of killing or reducing the number of target cells in a patient which target cells aberrantly express a polypeptide comprising an antigenic peptide according to the present invention or a corresponding human reference peptide, the method comprising administering to the patient an effective number of T cells as produced according to the present invention.

The present invention further relates to the use of any peptide as described, the nucleic acid according to the present invention, the expression vector according to the present invention, the cell according to the present invention, the activated T lymphocyte, the T cell receptor or the antibody or other peptide- and/or peptide-MHC-binding molecules according to the present invention as a medicament or in the manufacture of a medicament. Preferably, the medicament is active against cancer.

In some embodiments, said medicament is for a cellular therapy, a vaccine or a protein based on a soluble TCR or antibody.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1: shows for Example 1 in vitro affinity for the antigenic peptide CDC20-B1 (ENT204_B1) in comparison to the corresponding human CDC20 epitope CDC20-H1 (ENT204-H).

FIG. 2: shows for Example 1 in vitro affinity for the antigenic peptide KIF2C-B1 (ENT207_B1) in comparison to the corresponding human KIF2C epitope KIF2C-H1 (ENT207-H).

FIG. 3: shows for Example 1 in vitro affinity for the antigenic peptide UBE2C-B1 (ENT168_B1) in comparison to the corresponding human UBE2C epitope UBE2C-H1 (ENT168-H) and to another human UBE2C epitope UBE2C-H11 (ENT168-HL).

FIG. 4: shows for Example 1 in vitro affinity for the antigenic peptide ANKRD30A-B1 (ENT169_B1) in comparison to the corresponding human ANKRD30A epitope ANKRD30A-H1 (ENT169-H).

FIG. 5: shows for Example 1 in vitro affinity for the antigenic peptide CDH17-B1 (ENT176_B1) in comparison to the corresponding human CDH17 epitope CDH17-H1 (ENT176-H).

FIG. 6: shows for Example 1 in vitro affinity for the antigenic peptide TOP2A-B2 (ENT205_31) in comparison to the corresponding human TOP2A epitope TOP2A-H2 (ENT205-H).

FIG. 7: shows for Example 2 ELISPOT results for HHD DR1 HLA-A2 transgenic mice vaccinated with the antigenic peptide CDC20-B1 (ENT204_61) as indicated in the figure and cross-reactivity with the human corresponding peptide CDC20-H1 (ENT204-H). The data were provided as a number of spots per 1·10⁶ total T cells.

FIG. 8: shows for Example 2 ELISPOT results for HHD DR1 HLA-A2 transgenic mice vaccinated with the antigenic peptide KIF2C-B1 (ENT207_B1) as indicated in the figure and cross-reactivity with the human corresponding peptide KIF2C-H1 (ENT207-H). The data were provided as a number of spots per 1·10⁶ total T cells.

FIG. 9: shows for Example 2 ELISPOT results for HHD DR1 HLA-A2 transgenic mice vaccinated with the antigenic peptide UBE2C-B1 (ENT168_31) as indicated in the figure and cross-reactivity with the human corresponding peptide UBE2C-H1 (ENT168-H) and to another human UBE2C epitope UBE2C-H11 (ENT168-HL). The data were provided as a number of spots per 1·10⁶ total T cells.

FIG. 10: shows for Example 2 ELISPOT results for HHD DR1 HLA-A2 transgenic mice vaccinated with the antigenic peptide ANKRD30A-B1 (ENT169_B1) as indicated in the figure and cross-reactivity with the human corresponding peptide ANKRD30A-H1 (ENT169-H). The data were provided as a number of spots per 1·10⁶ total T cells

FIG. 11: shows for Example 2 ELISPOT results for HHD DR1 HLA-A2 transgenic mice vaccinated with the antigenic peptide CDH17-B1 (ENT176_B1) as indicated in the figure and cross-reactivity with the human corresponding peptide CDH17-H1 (ENT176-H). The data were provided as a number of spots per 1·10⁶ total T cells.

FIG. 12: shows for Example 2 ELISPOT results for HHD DR1 HLA-A2 transgenic mice vaccinated with the antigenic peptide TOP2A-B2 (ENT205_13) as indicated in the figure and cross-reactivity with the human corresponding peptide TOP2A-H2 (ENT205-H). The data were provided as a number of spots per 1·10⁶ total T cells.

FIG. 13: shows for Example 3 the detection of CDC20-B1, KIF2C-B1 and UBE2C-B1 peptide-specific CD8+ T cells detected in peripheral blood from healthy donors (HLA-A2 positive).

FIG. 14: shows for Example 1 in vitro affinity for the antigenic peptide KIF2C-B11 (ENT207_B2) in comparison to the corresponding human KIF2C epitope KIF2C-H1 (ENT207-H).

FIG. 15: shows for Example 1 in vitro affinity for the antigenic peptide KIF2C-B12 (ENT207_B3) in comparison to the corresponding human KIF2C epitope KIF2C-H1 (ENT207-H).

FIG. 16: shows for Example 1 in vitro affinity for the antigenic peptide KIF2C-B13 (ENT207_B4) in comparison to the corresponding human KIF2C epitope KIF2C-H1 (ENT207-H).

FIG. 17: shows for Example 1 in vitro affinity for the antigenic peptide UBE2C-B11 (ENT168_B2) in comparison to the corresponding human UBE2C epitope UBE2C-H1 (ENT168-H).

FIG. 18: shows for Example 3 the cytotoxic capacity of the CDC20-B1, KIF2C-B1 and UBE2C-B1 peptide-specific human T cells clone expanded in vitro by microbiome derived peptide stimulation. CDC20-B1, K12C-B1 and UBE2C-B1 peptide-specific T cells have the ability to kill T2 cells loaded with bacterial or human peptides.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Antigenic peptides have superior affinity to the HLA-A*0201 allele

Next, binding affinity of various selected antigenic peptides and of the corresponding fragments of human tumor antigens (human reference peptides) to the HLA-A*0201 allele was confirmed in vitro. Namely, the antigenic peptide of sequence SEQ ID NO: 1 («SLPDRILTV»; also referred herein as CDC20-B1) was compared to the corresponding reference human peptide derived from CDC20 («SLPDRILDA»; SEQ ID NO: 17, also referred herein as CDC20-H1). Moreover, the antigenic peptide of sequence SEQ ID NO: 2 («ALNPELLAL»; also referred herein as KIF2C-B1) was compared to the corresponding reference human peptide derived from KIF2C («AINPELLQL»; SEQ ID NO: 18, also referred herein as KIF2C-H1). Moreover, the antigenic peptide of sequence SEQ ID NO: 3 («FLAFVPLQL»; also referred herein as UBE2C-B1) was compared to the corresponding reference human peptides derived from UBE2C («ALYDVRTIL»; SEQ ID NO: 19, also referred herein as UBE2C-H1; and «ALYDVRTILL», SEQ ID NO: 38, also referred herein as UBE2C-H11). Moreover, the antigenic peptide of sequence SEQ ID NO: 4 («YLAFVPLAL»; also referred herein as UBE2C-B11) was compared to the corresponding reference human peptides derived from UBE2C («ALYDVRTIL»; SEQ ID NO: 19, also referred herein as UBE2C-H1).

Moreover, the antigenic peptide of sequence SEQ ID NO: 5 («SLLSlQSYV»; also referred herein as UBE2C-B2) was compared to the corresponding reference human peptide derived from UBE2C («ILLSIQSLLP», SEQ ID NO: 20, also referred herein as UBE2C-H2). Moreover, the antigenic peptide of sequence SEQ ID NO: 6 («YLQQELMNL»; also referred herein as UBE2C-B3) was compared to the corresponding reference human peptide derived from UBE2C («RLQQELMTL», SEQ ID NO: 21, also referred herein as UBE2C-H3). Moreover, the antigenic peptide of sequence SEQ ID NO: 7 («ALYSEILTV»; also referred herein as ANKRD30A-B1) was compared to the corresponding reference human peptide derived from ANKRD30A («AVYSEILSV», SEQ ID NO: 22, also referred herein as ANKRD30A-H1).

Moreover, the antigenic peptide of sequence SEQ ID NO: 8 («LILDTVHSL»; also referred herein as ANKRD30A-B2) was compared to the corresponding reference human peptide derived from ANKRD30A («KILDTVHSC«, SEQ ID NO: 23, also referred herein as ANKRD30A-H2). Moreover, the antigenic peptide of sequence SEQ ID NO: 9 («TLDQKLFMV»; also referred herein as ANKRD30A-B3) was compared to the corresponding reference human peptide derived from ANKRD30A («SLDQKLFQL», SEQ ID NO: 24, also referred herein as ANKRD30A-H3). Moreover, the antigenic peptide of sequence SEQ ID NO: 10 («YLILEYATV»; also referred herein as AURKA-B1) was compared to the corresponding reference human peptide derived from AURKA («YLILEYAPL», SEQ ID NO: 25, also referred herein as AURKA-H1). Moreover, the antigenic peptide of sequence SEQ ID NO: 11 («KIIGIILAV»; also referred herein as CDH17-B1) was compared to the corresponding reference human peptide derived from CDH17 («LVIGlILAV», SEQ ID NO: 26, also referred herein as CDH17-H1). Moreover, the antigenic peptide of sequence SEQ ID NO: 12 («YLSGANLFV»; also referred herein as CEACAM5-B1) was compared to the corresponding reference human peptide derived from CEACAM5 («YLSGANLNL», SEQ ID NO: 27, also referred herein as CEACAM5-H1). Moreover, the antigenic peptide of sequence SEQ ID NO: 13 («IVWSDVTYV»; also referred herein as MMP11-B1) was compared to the corresponding reference human peptide derived from MMP11 («KVWSDVTPL», SEQ ID NO: 28, also referred herein as MMP11-H1). Moreover, the antigenic peptide of sequence SEQ ID NO: 14 («AVIGIVAAV»; also referred herein as OR51 E2-B1) was compared to the corresponding reference human peptide derived from OR51 E2 («AQIGIVAVV», SEQ ID NO: 29, also referred herein as OR51E2-H1). Moreover, the antigenic peptide of sequence SEQ ID NO: 16 («ALIFGQLLL»; also referred herein as TOP2A-B2 was compared to the corresponding reference human peptide derived from TOP2A («AlIFGQLLT», SEQ ID NO: 31, also referred herein as TOP2A-H2).

### A. Materials and Methods

### A1. Measuring the Affinity of the Peptide to T2 Cell Line.

The experimental protocol is similar to the one that was validated for peptides presented by the HLA-A*0201 (Tourdot et al., A general strategy to enhance immunogenicity of low-affinity HLA-A2.1-associated peptides: implication in the identification of cryptic tumor epitopes. Eur J Immunol. 2000 December; 30(12):3411-21). Affinity measurement of the peptides is achieved with the human tumoral cell T2 which expresses the HLA-A*0201 molecule, but which is TAP1/2 negative and incapable of presenting endogenous peptides.

T2 cells (5·10⁴ cells per well) are incubated with decreasing concentrations of peptides from 100 μM to 0.1 μM (4 points: 100 μM, 10 μM, 1 μM, 0.1 μM) in serum-free medium (TexMacs) supplemented with 100 ng/μl of 2 Microglobulin at 37° C. for 16 hours. Cells are then washed two times and marked with the anti-HLA-A2 antibody coupled to PE (clone REA517, Miltenyi).

The analysis is achieved by FACS (Macsquant analyzer 10 or a Macsquant analyzer 16-Miltenyi).

For each peptide concentration, the geometric mean of the labelling associated with the peptide of interest is subtracted from background noise and reported as a percentage of the geometric mean of the HLA-A*0202 labelling obtained for the reference peptide HIV pol 589-597 at a concentration of 100 μM. The relative affinity is then determined as follows: relative affinity=concentration of each peptide inducing 20% of expression of HLA-A*0201/concentration of the reference peptide inducing 20% of expression of HLA-A*0201.

### A2. Solubilisation of Peptides

Each peptide is solubilized by taking into account the amino acid composition. For peptides which do not include any Cystein, Methionin, or Tryptophane, the addition of DMSO is possible to up to 10% of the total volume. Other peptides are resuspended in water or NH₄OH.

### B. Results

The mean relative fluorescence intensity values (data are normalized to the mean fluorescence of HIV peptide, i.e. a value of 100 is equal to the best binding observed with HIV peptide) of T2 cells obtained for the various concentrations of each peptide are shown in Table 3 below:

TABLE 3

| Peptide | | | | | |
|---|---|---|---|---|---|
| | SEQ ID | | | | |
| Name | NO. | 100 | 10 | 1 | 0.1 |
| CDC20-B1 (ENT204_B1) | 1 | 105.9 | 89.5 | 36.1 | 3.9 |
| CDC20-H1 (ENT204-H) | 17 | 80.7 | 35.5 | 3.2 | 0.0 |
| KIF2C-B1 (ENT207_B1) | 2 | 103.7 | 77.8 | 10.7 | 0.0 |
| KIF2C-B11 (ENT207_B2) | 40 | 78.3 | 63.4 | 20.4 | 7.9 |
| KIF2C-B12 (ENT207_B3) | 41 | 67.7 | 84.7 | 41.4 | 9.8 |
| KIF2C- B13 (ENT207_B4) | 42 | 50.3 | 45.3 | 42.1 | 10.5 |
| KIF2C-H1 (ENT207-H) | 18 | 64.9 | 9.5 | 0.4 | 0.0 |
| UBE2C-B1 (ENT168_B1) | 3 | 121.6 | 33.4 | 7.9 | 4.5 |
| UBE2C-B11 (ENT168_B2) | 4 | 87.5 | 68.4 | 22.2 | 6.6 |
| UBE2C-H1 (ENT168-H) | 19 | 134.2 | 6.8 | −0.2 | 3.8 |
| UBEC2-H11 (ENT168-HL) | 38 | 135.0 | 8.9 | −2.3 | 4.2 |
| UBE2C-B2 (ENT172_B1) | 5 | N.D | 92.0 | 52.0 | 14.2 |
| UBE2C-H2 | 20 | N.D | 32.7 | 8.5 | 2.1 |
| UBE2C-B3 (ENT178_B1) | 6 | N.D | 105.9 | 65.8 | 18.1 |
| UBE2C-H3 | 21 | 82.4 | 71.5 | 12.3 | 1.3 |
| ANKRD30A-B1 (ENT169_B1) | 7 | 88.2 | 93.7 | 77.2 | 33.3 |
| ANKRD30A-H1 (ENT169-H) | 22 | 103.6 | 80.1 | 29.9 | 3.0 |
| ANKRD30A-B2 (ENT174_B1) | 8 | 90.8 | 72.0 | 12.4 | 1.7 |
| ANKRD30A-H2 | 23 | 52.3 | 8.7 | 1.8 | 1.2 |
| ANKRD30A-B3 (ENT180_B1) | 9 | 112.2 | 119.7 | 67.5 | 18.8 |
| ANKRD30A-H3 | 24 | 97.1 | 99.1 | 41.9 | 5.9 |
| AURKA-B1 (ENT199_B1) | 10 | 105.7 | 65.4 | 26.6 | 4.0 |
| AURKA-H1 | 25 | 164.3 | 13.1 | 0.0 | 0.0 |
| CDH17-B1 (ENT176_B1) | 11 | N.D | 104.5 | 58.0 | 12.0 |
| CDH17-H1 (ENT176-H) | 26 | N.D | 17.6 | 3.7 | 0.5 |
| CEACAM-B1 (ENT188_B1) | 12 | 76.0 | 69.2 | 30.8 | 7.7 |
| CEACAM-H1 | 27 | 80.7 | 61.9 | 19.2 | 2.8 |
| MMP11-B1 (ENT187_B1) | 13 | 80.2 | 97.1 | 73.3 | 19.8 |
| MMP11-H1 | 28 | 82.5 | 72.7 | 24.9 | N.D |
| OR51E2-B1 (ENT190_B1) | 14 | N.D | 59.3 | 25.4 | 3.5 |
| OR51E2-H1 | 29 | N.D | 32.0 | 4.0 | 0.0 |
| TOP2A-B2 (ENT205_B1) | 16 | 145.5 | 73.6 | 5.5 | 3.4 |
| TOP2A-H2 (ENT205-H) | 31 | 0.0 | 3.2 | 0.3 | 5.1 |

Table 4 below summarizes for each tested peptide the concentration required to induce 20% of HLA-A2 expression and the in vitro binding affinity (* normalized against HIV-pol concentration of peptide inducing 20% of HLA-A2 expression performed during the same experiment).

TABLE 4

| | | Concentration | |
|---|---|---|---|
| | | of peptide that | |
| | | induces 20% | |
| | | of HLA-A2 | In vitro |
| | SEQ ID | expression | binding |
| Peptide | NO | (μM) | affinity* |
| CDC20-B1 (ENT204_B1) | 1 | 0.46 | 0.6 |
| CDC20-H1 (ENT204-H) | 17 | 4.57 | 6.3 |
| KIF2C-B1 (ENT207_B1) | 2 | 1.15 | 1.6 |
| KIF2C-B11 (ENT207_B2) | 40 | 0.909 | 3.9 |
| KIF2C-B12 (ENT207_B3) | 41 | 0.258 | 1.1 |
| KIF2C-B13 (ENT207_B4) | 42 | 0.189 | 0.8 |
| KIF2C-H1 (ENT204-H) | 18 | 22.55 | 31.1 |
| UBE2C-B1 (ENT168_B1) | 3 | 5.30 | 3.1 |
| UBE2C-B11 (ENT168_B2) | 4 | 0.87 | 0.7 |
| UBE2C-H1 (ENT168-H) | 19 | 14.98 | 8.8 |
| UBEC2-H11 (ENT168-HL) | 38 | 14.87 | 8.7 |
| UBE2C-B2 (ENT172_B1) | 5 | 0.22 | 0.2 |
| UBE2C-H2 | 20 | 3.27 | 3.5 |
| UBE2C-B3 (ENT178_B1) | 6 | 0.14 | 0.2 |
| UBE2C-H3 | 21 | 1.06 | 1.1 |
| ANKRD30A-B1 (ENT169_B1) | 7 | 0.05 | 0.04 |
| ANKRD30A-H1 (ENT169-H) | 22 | 0.65 | 0.5 |
| ANKRD30A-B2 (ENT174_B1) | 8 | 1.13 | 1.3 |
| ANKRD30A-H2 | 23 | 25.32 | 29.9 |
| ANKRD30A-B3 (ENT180_B1) | 9 | 0.14 | 0.2 |
| ANKRD30A-H3 | 24 | 0.32 | 0.4 |
| AURKA-B1 (ENT199_B1) | 10 | 1.16 | 2.2 |
| AURKA-H1 | 25 | 12.2 | 22.8 |
| CDH17-B1 (ENT176_B1) | 11 | 0.2 | 0.2 |
| CDH17-H1 (ENT176-H) | 26 | 14.06 | 14.9 |
| CEACAM-B1 (ENT188_B1) | 12 | 0.5 | 0.5 |
| CEACAM-H1 | 27 | 1.07 | 1.2 |
| MMP11-B1 (ENT187_B1) | 13 | 0.08 | 0.07 |
| MMP11-H1 | 28 | 0.65 | 0.6 |
| OR51E2-B1 (ENT190_B1) | 14 | 0.7 | 0.8 |
| OR51E2-H1 | 29 | 5.68 | 6.2 |
| TOP2A-B2 (ENT205_B1) | 16 | 1.79 | 2.9 |
| TOP2A-H2 (ENT205-H) | 31 | N.D | N.D |

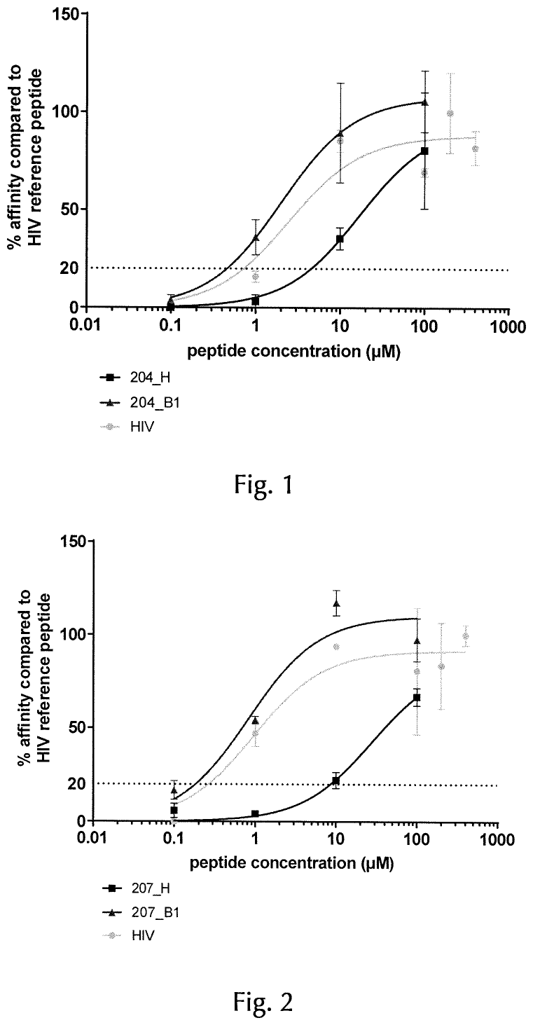

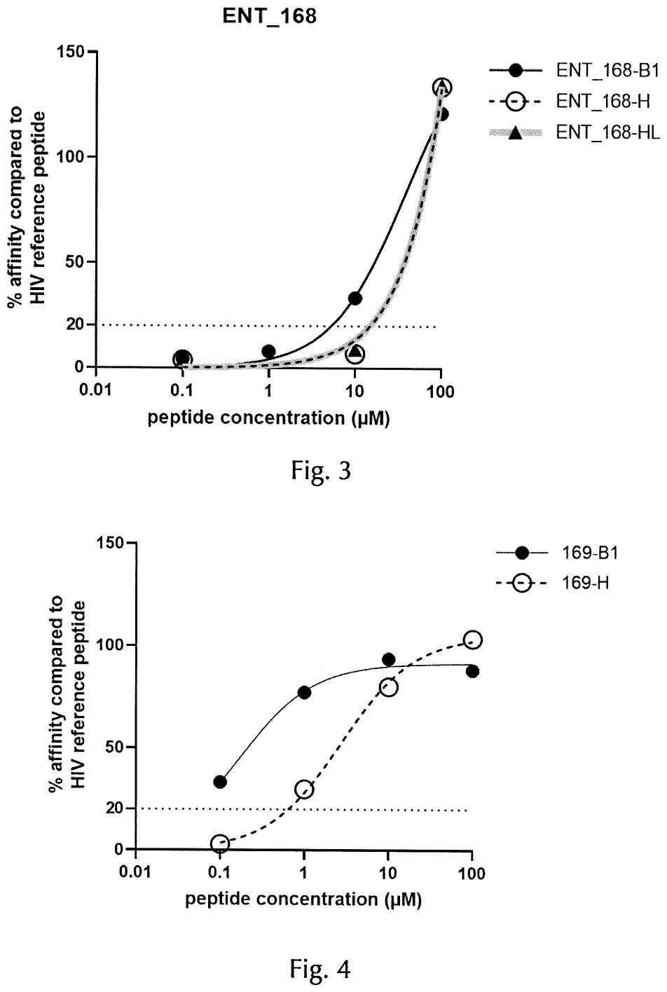

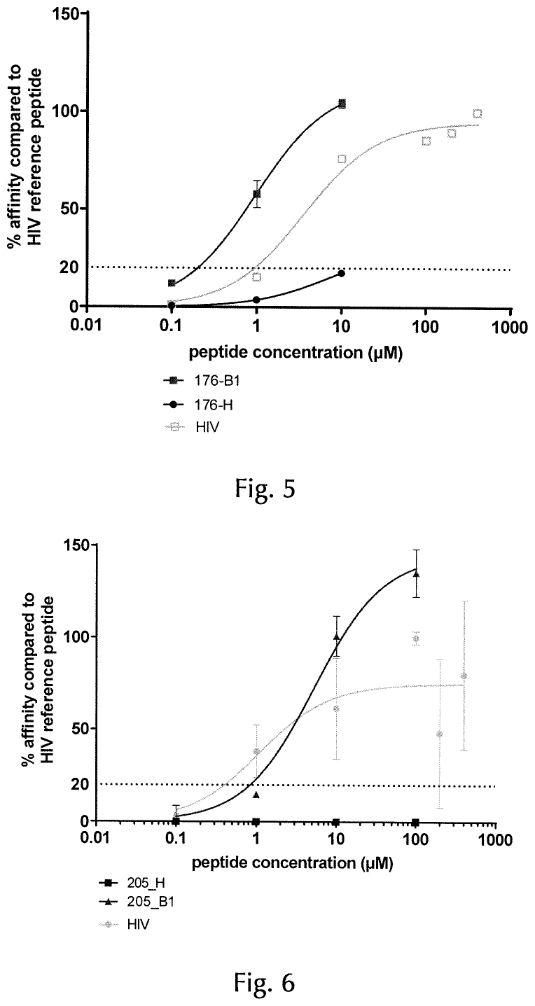

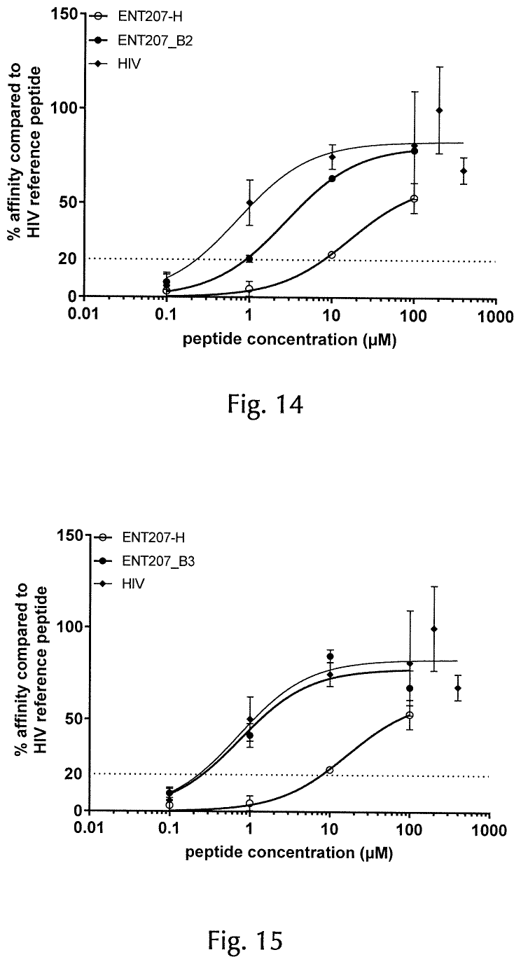

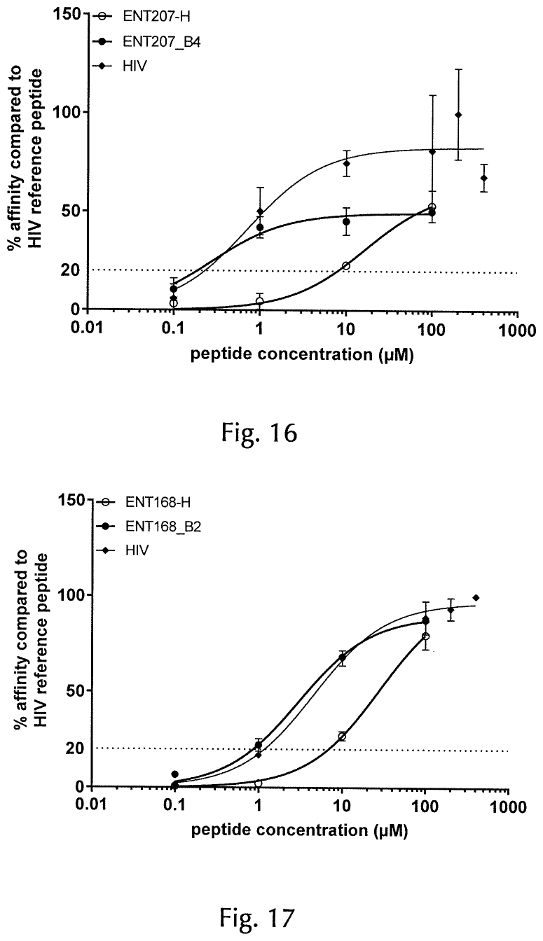

In addition, FIGS. 1-6 and 14-17 illustrate the results for selected examples, namely for the antigenic peptide CDC20-B1 in comparison to the corresponding human CDC20 fragment CDC20-H1 (FIG. 1), for the antigenic peptide KIF2C-B1, in comparison to the corresponding human KIF2C fragment KIF2C-H1 (FIG. 2), for the antigenic peptide KIF2C-811 in comparison to the corresponding human KIF2C fragment KIF2C-H1 (FIG. 14), for the antigenic peptide KIF2C-812 in comparison to the corresponding human KIF2C fragment KIF2C-H1 (FIG. 15), for the antigenic peptide KIF2C-B13 in comparison to the corresponding human KIF2C fragment KIF2C-H1 (FIG. 16), for the antigenic peptide UBE2C-B1 in comparison to the corresponding human UBE2C fragment UBE2C-H1 and to another human UBE2C epitope UBE2C-H11 (FIG. 3), for the antigenic peptide UBE2C-B11 in comparison to the corresponding human UBE2C fragment UBE2C-H1 (FIG. 17), for the antigenic peptide ANKRD30A-B1 in comparison to the corresponding human ANKRD30A fragment ANKRD30A-H1 (FIG. 4), for the antigenic peptide CDH17-B1 in comparison to the corresponding human CDH17 fragment CDH17-H1 (FIG. 5), for the antigenic peptide TOP2A-B2 in comparison to the corresponding human TOP2A fragment TOP2A-H2 (FIG. 6).

In summary, the results show that the antigenic peptides according to the present invention show at least similar binding affinity to HLA-A*0201 as the corresponding human tumor antigen fragments. In most cases, the binding affinity observed for the antigenic peptides according to the present invention was stronger than that of the corresponding human epitopes. Without being bound to any theory it is assumed that such a strong binding affinity of the antigenic peptides according to the present invention reflects their ability to raise an immune response (i.e., their immunogenicity).

### Example 2: Immunogenicity of UBE2C-B1 (ENT 168-B1). ANKRD30A-B1 (ENT 169-B1). CDH17-B1 (ENT 176-B1). CDC20-B1 (ENT 204-B1). TOP2A-B2 (ENT 205-B1) and KIF2C-B1 (ENT 207-B1) in HLA-A2 Transgenic Mice and Cross-Reactivity with the Corresponding Human Peptide

### A. Materials and Methods

### A.1 Mouse Model

Briefly, HLA-A2 HHD-DR1 humanized mice (C57BL/6JB2mtm1UnclAb−/−Tg(HLA-DRA, HLA-DRB1*0101)#GjhTg(HLA-A/H2-D/B2M)1 Bpe) were assigned randomly (based on mouse sex and age) to experimental groups, wherein each group was immunized with a specific vaccination peptide (vacc-pAg) combined to a common helper peptide (h-pAg UCP2; sequence: KSVWSKLQSIGIRQH; SEQ ID NO: 39) (as outlined in Table 6 below).

TABLE 6
Experimental group composition.

| | Peptide | Helper | | | | Animal |
|---|---|---|---|---|---|---|
| Group | (vacc-pAg) | (h-Ag) | Mice | Prime | Boost | Number |
| 1 | UBE2C-B1 | UCP2 | HHD- | + | +(1X) | 5 |
| | ENT_168-B1 | (100 μg per | DR1 | | | |
| | (30 nmol per | mouse) | | | | |
| | mouse) | | | | | |
| 2 | ANKRD30A-B1 | UCP2 | HHD- | + | +(1X) | 5 |
| | ENT_169-B1 | (100 μg per | DR1 | | | |
| | (30 nmol per | mouse) | | | | |
| | mouse) | | | | | |
| 3 | CDH17-B1 | UCP2 | HHD- | + | +(1X) | 5 |
| | ENT_176-B1 | (100 μg per | DR1 | | | |
| | (30 nmol per | mouse) | | | | |
| | mouse) | | | | | |
| 4 | CDC20-B1 | UCP2 | HHD- | + | +(1X) | 5 |
| | ENT_204-B1 | (100 μg per | DR1 | | | |
| | (30 nmol per | mouse) | | | | |
| | mouse) | | | | | |
| 5 | TOP2A-B2 | UCP2 | HHD- | + | +(1X) | 5 |
| | ENT_205-B1 | (100 μg per | DR1 | | | |
| | (30 nmol per | mouse) | | | | |
| | mouse) | | | | | |
| 6 | KIF2C-B1 | UCP2 | HHD- | + | +(1X) | 5 |
| | ENT_207-B1 | (100 μg per | DR1 | | | |
| | (30 nmol per | mouse) | | | | |
| | mouse) | | | | | |

h-pAg: ‘helper’ peptide;
vacc-pAg: vaccination peptide.
The number of boost injections is indicated into brackets.

The peptides were provided as follows:
vacc-pAg: UBE2C-B1, ANKRD30A-B1, CDH1 7-B1, CDC20-B1, TOP2A-B2 and KIF2C-B1 all produced and provided at a 4 mM concentration;
h-pAg: UCP2 re-suspended in pure distilled water at a 10 mg/mL concentration

The peptide formulation to be injected (emulsion) was prepared freshly on each day of injection and for each group. A mix for 10 animals was prepared using 2 mL luer lock syringes (4606701 V, B BRAUN) and luer lock female-female Combifix® Adapter (B. Braun, 5206634): 500 μL of peptide mixture in syringe 1 was emulsified with 500 μL of Montanide ISA 51 VG (Seppic) contained in syringe 2. The emulsification process was performed first by pushing the peptide mixture contained in syringe 1 into syringe 2 (containing Montanide) at very low speed. Transfer of the mixture from one syringe to another was then performed at very low speed during 6 cycles (one cycle corresponding to one passage from one syringe to another), and secondly at high speed, as fast as possible, for 1 minute. Each emulsion was prepared in excess to compensate for the dead volumes at injection.

The animals were immunized on day 0 (d0) with a prime injection, and on d14 with a boost injection. Each mouse was injected s.c. on the loose skin over the neck of the animals with 100 μL of an oil-based emulsion that contained:
30 nmol of vacc-pAg and 30 or 100 μg of UCP2 helper peptide in 50 μL of a dedicated final solvent
Montanide ISA 51 VG (Seppic) added at 1:1 (v:v) ratio (50 μL per mouse).

### A.2 Analysis

Seven days after the boost injection (i.e. on d21), the animals were euthanized and the spleen was harvested. Splenocytes were prepared by mechanical disruption of the organ followed by 70 μm-filtering and Red blood cell lysis.

The cell suspensions were further used in an ELISPOT-IFN assay (Table 5). The cells were cultured in 200 μL of complete T cell medium. Experimental conditions (duplicates) were as follow: 2×10⁵ total cells per well when cultured in presence of various pAg (10 μM) or medium-only; and 2×10⁴ total cells when cultured in presence of the positive control PMA/lonomycin (PMA: Sigma P8139: 0.1 μM final; lonomycin: Sigma 10634: 1 μM final). The cultures were assessed for their capacity to secrete IFN (mouse IFN-ELISpotPLUS kit, Mabtech 3321-4APT-10), following the manufacturer's instructions (˜12-48h incubation time before performing the assay). The peptides used for restimulation are described in Table 7.

TABLE 7
Setup of the ELISPOT-IFNγ assay.

| Group | Stimulus | Wells | Animal | Total |
|---|---|---|---|---|
| 1 | UBE2C-H1 (10 μM) | 3 | 6 | 18 |
| | UBE2C-H11 (10 μM) | 3 | 6 | 18 |
| | UBE2C-B1 (10 μM) | 3 | 6 | 18 |
| | CD3/CD28 bead | 3 | 6 | 18 |
| | Medium | 3 | 6 | 18 |
| 2 | ANKRD30A-H1 (10 μM) | 3 | 6 | 18 |
| | ANKRD30A-B1 (10 μM) | 3 | 6 | 18 |
| | CD3/CD28 bead | 3 | 6 | 18 |
| | Medium | 3 | 6 | 18 |
| 3 | CDH17-H1 (10 μM) | 3 | 6 | 18 |
| | CDH17-B1 (10 μM) | 3 | 6 | 18 |
| | CD3/CD28 bead | 3 | 6 | 18 |
| | Medium | 3 | 6 | 18 |
| 4 | CDC20-H1 (10 μM) | 3 | 6 | 18 |
| | CDC20-B1 (10 μM) | 3 | 6 | 18 |
| | CD3/CD28 bead | 3 | 6 | 18 |
| | Medium | 3 | 6 | 18 |
| 5 | TOP2A-H2 (10 μM) | 3 | 6 | 18 |
| | TOP2A-B2 (10 μM) | 3 | 6 | 18 |
| | CD3/CD28 bead | 3 | 6 | 18 |
| | Medium | 3 | 6 | 18 |
| 6 | KIF2C-H1 (10 μM) | 3 | 6 | 18 |
| | KIF2C-B1 (10 μM) | 3 | 6 | 18 |
| | CD3/CD28 bead | 3 | 6 | 18 |
| | Medium | 3 | 6 | 18 |

Spots were counted on a an iSpot Fluorospot Reader System (AID). Data plotting and statistical analysis were performed with the Prism-9 software (GraphPad Software Inc.).

### B. Results

All mice were aged from 8 to 15 weeks at the experiment starting date. Both males and females were used in the study. Animals have been housed in groups of 6 per cage at maximum. At time of sacrifice, the spleen T cell population was analysed by flow cytometry, showing that the large majority belonged to the CD4+ T cell subset.

After plating and incubation with the appropriate stimuli, the IFNγ-producing cells were revealed and counted. The data were provided as a number of spots per 1·10⁶ total T cells. The individual average values (obtained from the duplicates) were next used to plot the group average values. Statistical analysis for comparison (to the irrelevant peptide condition) were performed using unpaired non-parametric test (Mann Whitney) (**: p<0.01; *: p<0.05).

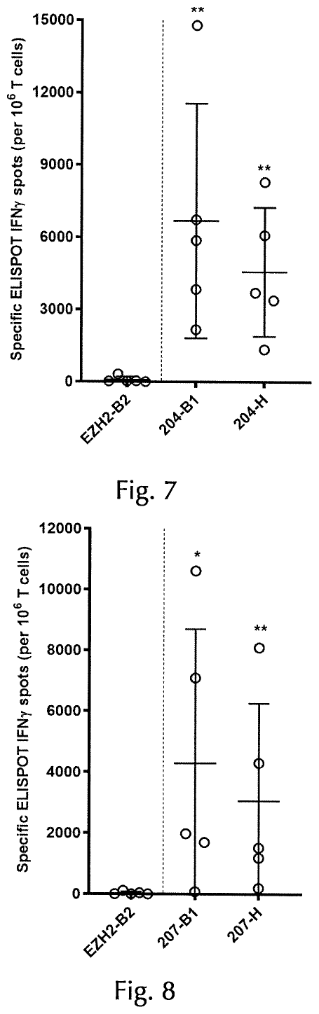

Overall, vaccination with the antigenic peptides according to the present invention (CDC20-B1, KIF2C-B1, UBE2C-B1, ANKRD30A-B1, CDH17-B1 and TOP2A-B2) induced significant T cell responses in the ELISPOT-IFNγ assay in HHD DR1 mice (FIGS. 7-12).

The results (FIG. 7) show that immunization of HHD-DR1 mice with CDC20-B1 allows to induce T-cells that are able to react strongly after challenge with either CDC20-B1 or the human corresponding peptide CDC20-H1. Thus, CDC20-B1 is strongly immunogenic and is able to drive an effective immune response against the corresponding human peptide.

The results (FIG. 8) show that immunization of HHD-DR1 mice with KIF2C-B1 allows to induce T-cells that are able to react strongly after challenge with either KIF2C-B1 or the human corresponding peptide KIF2C-H1. Thus, KIF2C-B1 is strongly immunogenic and is able to drive an effective immune response against the corresponding human peptide.

The results (FIG. 9) show that immunization of HHD-DR1 mice with UBE2C-B1 allows to induce T-cells that are able to react strongly after challenge with either UBE2C-B1 or the human corresponding peptide UBE2C-H1 and to another human UBE2C epitope UBE2C-H11. Thus, UBE2C-B1 is strongly immunogenic and is able to drive an effective immune response against the corresponding human peptide and to another human UBE2C epitope UBE2C-H11.

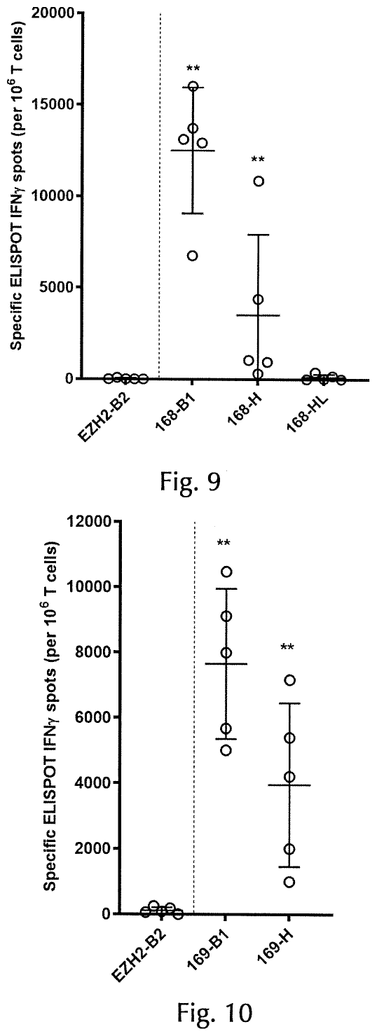

The results (FIG. 10) show that immunization of HHD-DR1 mice with ANKRD30A-B1 allows to induce T-cells that are able to react strongly after challenge with either ANKRD30A-B1 or the human corresponding peptide ANKRD30A-H1. Thus, ANKRD30A-B1 is strongly immunogenic and is able to drive an effective immune response against the corresponding human peptide.

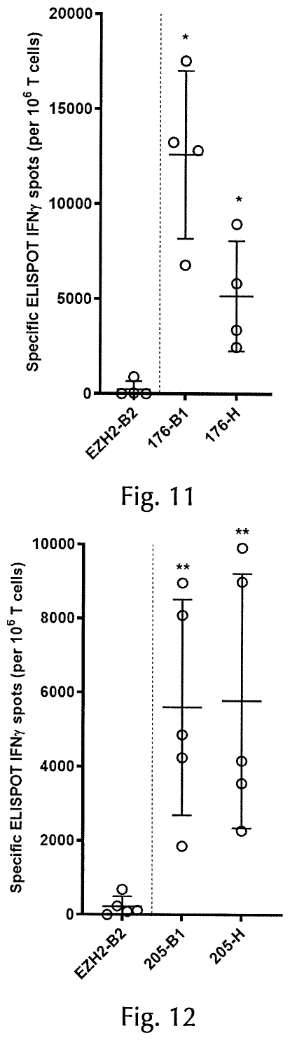

The results (FIG. 11) show that immunization of HHD-DR1 mice with CDH17-B1 allows to induce T-cells that are able to react strongly after challenge with either CDH17-B1 or the human corresponding peptide CDH17-H1. Thus, CDH17-B1 is strongly immunogenic and is able to drive an effective immune response against the corresponding human peptide.

The results (FIG. 12) show that immunization of HHD-DR1 mice with TOP2A-B2 allows to induce T-cells that are able to react strongly after challenge with either TOP2A-B2 or the human corresponding peptide TOP2A-H2. Thus, TOP2A-B2 is strongly immunogenic and is able to drive an effective immune response against the corresponding human peptide.

Altogether, these immunogenicity studies described in Examples 2 performed in HHD DR1 mice showed that the 6 antigenic peptides of the invention, CDC20-B1, KIF2C-B1, UBE2C-B1, ANKRD30A-B1, CDH17-B1 and TOP2A-B2 induced strong immune responses. Cross-reactivity of the T cells generated against CDC20-H1, KIF2C-B2, UBE2C-B1, ANKRD30A-B1, CDH17-B1 and TOP2A-B2 for the corresponding human peptides was shown in HHD DR1 mice.

Accordingly, those results provide experimental evidence that antigen-based immunotherapy is able to improve T cell response in vivo and that the antigenic peptides according to the present invention are particularly efficient for that purpose.

### Example 3: Ex Vivo Cytotoxic Effects of UBE2C-B1 (ENT 168-B1). CDC20-B1 (ENT 204-B1), KIF2C-B1 (ENT 207-61) Specific CD8 Human T Cells

Multiple investigations support the notion of presence of a repertoire of specific T cells against microbial peptides. The number of microbial specific T-cells against peptides is expected to be low, but sufficient to be re-activated by a vaccine challenge.

To identify and functionally characterize circulating UBE2C-B1 (ENT_168-61), CDC20-B1 (ENT_204-B1), and KIF2C-B1 (ENT_207-B1) specific T cells in humans, an in vitro amplification protocol has been developed in order to detect T cells specific for each antigenic peptide and investigate their cytotoxic capacity.

### 3.1 Identification of Antigenic Peptide-Specific CD8 T Cells in Human

In vitro amplification method and specific pMHC multimers have been used for identification of UBE2C-B1 (ENT_168-B1), CDC20-B1 (ENT_204-B1) and KIF2C-B1 (ENT_207-B1) specific T cells. pMHC multimers were generated for all the bacteria peptides and their respective human counterpart. PBMCs from several HLA-A*02 healthy donors (up to 13 donors) were collected, enriched after CD137 and CD8 selection and subjected to multiple rounds of in vitro amplification with E04010 peptides loaded T2 cells to increase the number of specific T cell clones. Detection of OMP peptide specific CD8 T cells using cytometry analysis with the fluorescent multimer was performed on enriched CD8 T cell populations

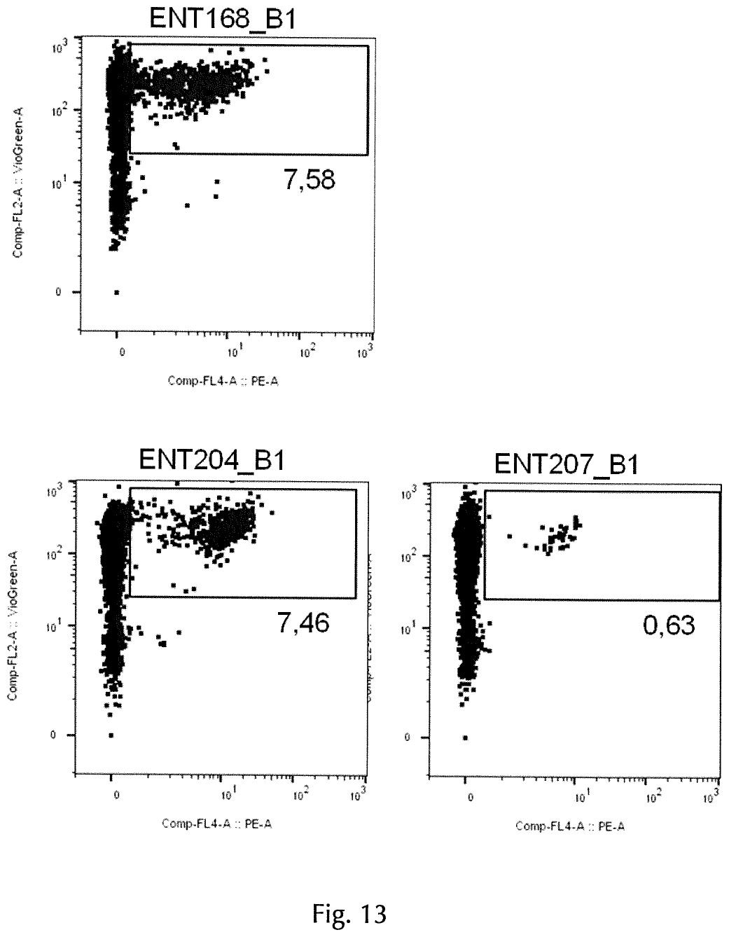

FIG. 13 exemplifies results obtained with one HLA-A2 healthy donor. For this donor, cell amplification allows detection of UBE2C-B1 (ENT_168-B1) specific cells (7.58%), CDC20-B1 (ENT_204-B1) specific cells (7.46%), and KIF2C-B1 (ENT_207-B1) specific cells (0.63%).

In conclusion, these results demonstrate the presence of CD8 T cells in the blood of healthy HLA-A2 donors that can recognize the microbiome-derived peptides.

### 3.2 Antigenic Peptide-Specific CD8 T Cytotoxicity Functions

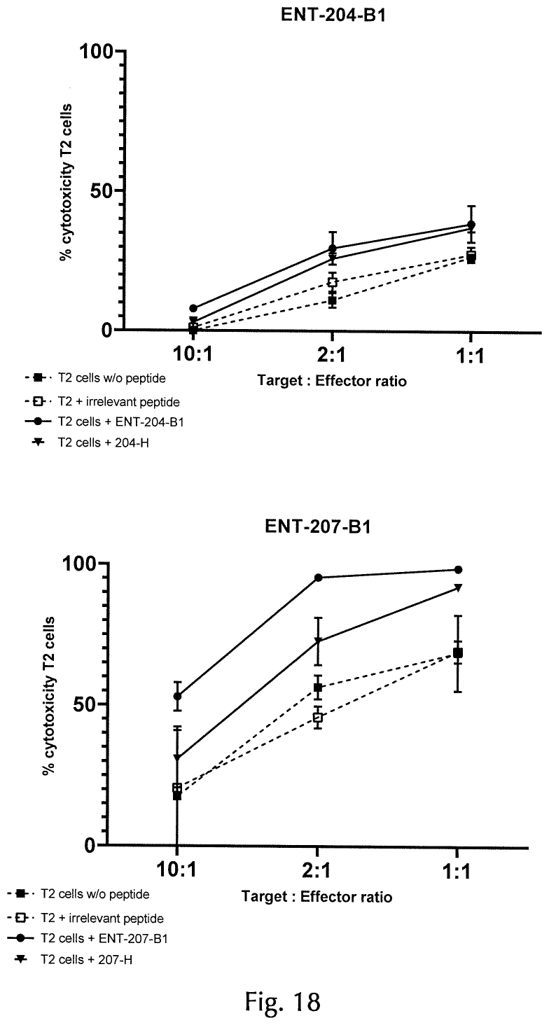

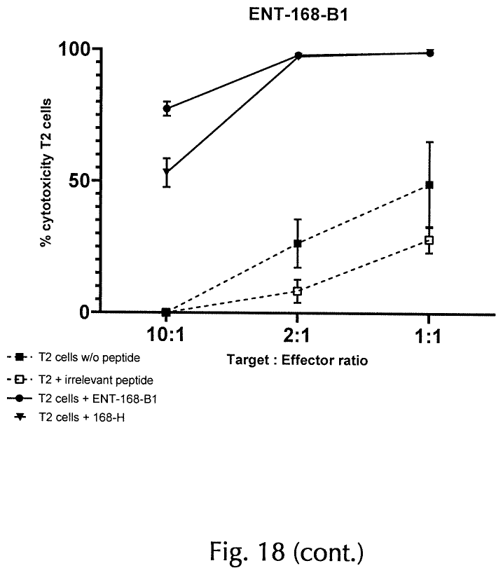

CD8+ T cells expanded per above were used to perform cytotoxic assays in presence of different ratios of target and effector cells to assess their cytotoxic capacity, using flow cytometry readout. Target cells were T2 cell lines loaded with bacterial peptide or human counterpart peptide. As a negative control T2 cells unloaded and T2 cells loaded with irrelevant peptide were employed. As shown in FIG. 18, antigenic peptide-specific human T cells clone expanded in vitro have the capacity to kill T2 cells loaded with all the bacteria peptide, UBE2C-B1 (ENT_168-B1), CDC20-B1 (ENT_204-B1), and KIF2C-B1 (ENT_207-B1). More importantly, UBE2C-B1 (ENT_168-B1), CDC20-B1 (ENT_204-B1), and KIF2C-B1 (ENT_207-B1) specific human T cells clones expanded in vitro were able to kill T2 cells loaded with the human UBEC2, CDC20 and KIF2C peptide.

Overall, these results demonstrate the presence of T cell clones in healthy volunteers able to recognize microbial peptide and to kill target with microbial peptides and human counterparts. These data are particularly encouraging as T cell clones have been obtained in healthy donors, therefore we could expect that specific T cell clones could be efficiently amplified in patients exposed to the immunization by antigenic peptides of the invention.

1-15. (canceled)

16. An antigenic peptide comprising an amino acid sequence as set forth in any one of SEQ ID NOs 1-16 and SEQ ID NOs 40-42.

17. The antigenic peptide according to claim 16, wherein the antigenic peptide consists of an amino acid sequence as set forth in any one of SEQ ID NOs 1-16 and SEQ ID NOs 40-42.

18. The antigenic peptide according to claim 16, wherein the antigenic peptide has a length of 8 to 15 amino acids.

19. The antigenic peptide according to claim 16, wherein the antigenic peptide comprises an amino acid sequence as set forth in SEQ ID NO: 1.

20. The antigenic peptide according to claim 16, wherein the antigenic peptide comprises an amino acid sequence as set forth in SEQ ID NO: 2.

21. The antigenic peptide according to claim 16, wherein the antigenic peptide comprises an amino acid sequence as set forth in SEQ ID NO: 3.

22. An immunogenic compound, wherein it comprises a polypeptide of formula (I)
PepNt-CORE-PepCt (I)
wherein:
“PepNt” consists of a polypeptide having a length varying from 0 to 500 amino acid residues and is located at the N-terminal end of the polypeptide of formula (I);
CORE consists of the antigenic peptide according to claim 17; and
“PepCt” consists of a polypeptide having a length varying from 0 to 500 amino acid residues and is located at the C-terminal end of the polypeptide of formula (I).

23. A nucleic acid encoding the antigenic peptide according to claim 16 or an immunogenic compound comprising it.

24. A pharmaceutical composition comprising:
the antigenic peptide according to claim 16, an immunogenic compound comprising it, a nucleic acid encoding it or a nucleic acid encoding an immunogenic compound comprising it; and
one or more pharmaceutically acceptable excipients or carriers.

25. A pharmaceutical composition comprising:
at least two distinct antigenic peptides according to claim 16, at least two distinct immunogenic compounds comprising each one of them, at least two distinct nucleic acids encoding each one of them; and
one or more pharmaceutically acceptable excipients or carriers.

26. The pharmaceutical composition according to claim 25, wherein said at least two distinct antigenic peptides are chosen in the list consisting of
an antigenic peptide comprising an amino acid sequence as set forth in SEQ ID NO: 1;
an antigenic peptide comprising an amino acid sequence as set forth in SEQ ID NO: 2; and
an antigenic peptide comprising an amino acid sequence as set forth in SEQ ID NO: 3.

27. The pharmaceutical composition according to claim 26, wherein the pharmaceutical composition further comprises
an antigenic peptide comprising an amino acid sequence as set forth in SEQ ID NO: 32; and
an antigenic peptide comprising an amino acid sequence as set forth in SEQ ID NO: 33, or an antigenic peptide comprising an amino acid sequence as set forth in SEQ ID NO: 34.

28. The pharmaceutical composition according to claim 27, wherein the pharmaceutical composition further comprises a helper peptide comprising an amino acid sequence according to SEQ ID NO: 39.

29. A peptide-MHC (pMHC) multimer comprising the antigenic peptide according to claim 16.

30. A pharmaceutical composition according to claim 26, wherein it comprises:
an antigenic peptide consisting of an amino acid sequence as set forth in SEQ ID NO: 1;
an antigenic peptide consisting of an amino acid sequence as set forth in SEQ ID NO: 2; and
an antigenic peptide consisting of an amino acid sequence as set forth in SEQ ID NO: 3.
an antigenic peptide consisting of an amino acid sequence as set forth in SEQ ID NO: 32;
an antigenic peptide consisting of an amino acid sequence as set forth in SEQ ID NO: 33, and
a helper peptide consisting of an amino acid sequence according to SEQ ID NO: 39.

31. The pharmaceutical composition according to claim 24, wherein it further comprises an immunostimulatory agent chosen in the list consisting of immune adjuvants, antigen-presenting cells, and combinations thereof, wherein the immune adjuvant is a helper peptide having immuno-adjuvant properties chosen in the list consisting of tetanus helper peptide, keyhole limpet hemocyanin peptide, PADRE peptide, HHD-DR3 peptide, h-pAg T13L or the UCP2 peptide of sequence the sequence SEQ ID NO: 39.

32. A method for preventing and/or treating a cancer or initiating, enhancing or prolonging an anti-tumor-response against a cancer in a subject in need thereof comprising administering to the subject the antigenic peptide according to claim 16.

33. A method for preventing and/or treating a cancer or initiating, enhancing or prolonging an anti-tumor-response against a cancer in a subject in need thereof comprising administering to the subject the pharmaceutical composition according to claim 26.

34. A method for preventing and/or treating a cancer or initiating, enhancing or prolonging an anti-tumor-response against a cancer in a subject in need thereof comprising administering to the subject the pharmaceutical composition according to claim 30.

35. A method for preventing and/or treating a cancer or initiating, enhancing or prolonging an anti-tumor-response against a cancer in a subject in need thereof comprising administering to the subject the pharmaceutical composition according to claim 31.
